(19) United States
(12) Patent Application Publication
(10) Pub. No.: US 2026/0076974 A1
(43) Pub. Date: Mar. 19, 2026

(54) COMPOSITIONS FOR PREVENTING AND TREATING INFECTION COMPRISING AN ARTIFICIAL SWEETENER

(71) Applicant: Brunel University London, Uxbridge (GB)

(72) Inventor: Ronan McCarthy, Uxbridge (GB)

(73) Assignee: Brunel University London, Uxbridge (GB)

(21) Appl. No.: 18/854,693

(22) PCT Filed: Apr. 12, 2023
(86) PCT No.: PCT/GB2023/050983

§ 371 Date: Oct. 7, 2024

(30) Foreign Application Priority Data
Apr. 13, 2022 (GB) 2205515.6
Oct. 12, 2022 (GB) 2215010.6

(51) Int. Cl. A61K 31/54; A61K 9/00; A61K 9/06; A61K 31/18; A61K 31/428; A61K 38/12; A61P 31/02

(52) U.S. Cl. CPC A61K 31/54; A61K 9/0014; A61K 9/06; A61K 31/18; A61K 31/428; A61K 38/12; A61P 31/02

(57) ABSTRACT

Compositions for preventing and treating bacterial infections include artificial sweeteners such as ace-k, saccharin and cyclamate. The compositions are particularly envisaged for topical administration for treating skin infections and for treating or preventing infections caused by Pseudomonas aeruginosa and/or Acinetobacter baumannii. Compositions including artificial sweeteners at sub-minimum inhibitory concentrations can also be used to disrupt virulence or increase the sensitivity of antibiotic-resistant bacteria to those antibiotics.

The present invention relates to compositions for treating and/or preventing infection. It further relates to compositions for enhancing the activity of an antibiotic.

Infectious diseases are a leading cause of deaths world-wide, accounting for 25% of all deaths annually. This number would be significantly greater if it were not for antibiotics. The discovery of penicillin over 80 years ago and its subsequent uptake by healthcare systems around the world revolutionised the treatment of bacterial infections. It marked the beginning of a golden age in antibiotic discovery with new classes of antibiotics being routinely discovered and saving millions of lives globally particularly in areas of the developing world.

However, since the beginning of the 1990s the rate of discovery has slowed to a near standstill. This lack of discovery has been compounded by the rapid emergence and spread of bacterial pathogens that exhibit resistance to multiple antibiotic treatments, including first line antibiotic treatments. This has led to an antibiotic resistance crisis with deaths attributed to antimicrobial resistance reaching 4.95 million in 2019 (Murray et al., 2022), and a predicted cumulative global cost of $100 trillion by 2050 (HM Government (2019)).

A 2018 report from the World Health Organisation placed Acinetobacter baumannii and Pseudomonas aeruginosa at the top of a global priority list of bacteria in urgent need of novel therapeutic intervention strategies. Research into antibiotic discovery is now a matter of global priority in order to maintain sustainable access to effective treatments for bacterial infections. The rise of antibiotic resistance is closely linked to their indiscriminate use particularly in the developing world, where many antibiotics can be acquired without the need for a prescription or clinical advice. The urgent need to identify new compounds with antibiotic properties has prompted scientists to explore new environments and approaches to identify potential therapeutics.

The present invention seeks to provide compositions for treating and/or preventing infection, and compositions for enhancing the activity of an antibiotic.

According to an aspect of the present invention, there is provided a composition including an active agent in an amount sufficient to inhibit bacterial growth and/or virulence, wherein the active agent is an artificial sweetener or a chemically related derivative thereof, for use in a method of treating and/or preventing infection.

According to another aspect of the present invention, there is provided a composition including an active agent in an amount sufficient to inhibit bacterial growth and/or virulence, wherein the active agent is ace-K, saccharin, cyclamate, sucralose, a sugar alcohol or a chemically related derivative thereof, for use in a method of treating and/or preventing infection.

According to an aspect of the present invention, there is provided a composition including an artificial sweetener or a derivative thereof in an amount sufficient to inhibit bacterial growth and/or disable a virulence mechanism for use in a method of treating and/or preventing infection.

According to another aspect of the present invention, there is provided a composition including ace-K, saccharin, cyclamate, sucralose, a sugar alcohol, and/or derivatives thereof in an amount sufficient to inhibit bacterial growth and/or disable a virulence mechanism for use in a method of treating and/or preventing infection.

According to another aspect of the present invention, there is provided a method of preventing and/or treating infection, including providing a composition including an artificial sweetener or a chemically related derivative thereof in an amount sufficient to inhibit bacterial growth and/or disable a virulence mechanism, and administering the composition to a patient in need thereof.

In particular, the method may be a method of treating or preventing a skin infection (such as a burn or laceration), a method of treating or preventing an infection associated with lung disease, or a method of treating or preventing bacteraemia and/or sepsis.

In all aspects, the patient may be a human patient or an animal patient.

In all aspects, the derivative may be a chemically related derivative, which may have been formed by modifying the structure of the sweetener. For example, the derivative may be a pharmaceutically acceptable salt.

The active agent in the composition may include ace-K, saccharin, sucralose, cyclamate, sucralose, a sugar alcohol (such as xylitol, mannitol, sorbitol, erythritol, maltitol and/or lactitol), and/or derivatives thereof.

The active agent may have a structure that includes a sulphonamide group. For example, ace-K, cyclamate and saccharin have structures that include a sulphonamide group. In some embodiments, the active agent may be modified to include a sulphonamide group, or an additional sulphonamide group.

Preferred active agents include ace-K, saccharin or cyclamate, or a chemically related derivative of any of these.

The infection may be a bacterial infection, for example, infection by Pseudomonas aeruginosa, Acinetobacter baumannii, Staphylococcus aureus, Enterococcus faecalis, Klebsiella pneumoniae, Stenotrophomonas maltophilia, and/or Enterobacter species. In particular, the infection may be an infection caused by Pseudomonas aeruginosa, Acinetobacter baumannii, Staphylococcus aureus, Stenotrophomonas maltophilia, and/or Enterobacter species.

The virulence mechanism may be biofilm formation and/or bacterial motility for example. The active agent may therefore inhibit either or both of these virulence mechanisms. The active agent may disrupt the bacterial membrane.

The composition may be formulated for delivery by any suitable method. For example, it may be formulated for application to a patient's skin, for oral administration, for inhalation, or for intravenous administration. In particular, it is envisaged formulating the composition for application to a patient's skin, for inhalation, or for intravenous administration. Topical application is particularly preferred.

According to another aspect of the present invention, there is provided a composition including ace-K, cyclamate, saccharin or a derivative thereof, for use in enhancing the activity of an antibiotic.

According to another aspect of the present invention, there is provided a composition including an active agent, wherein the active agent is ace-K, cyclamate, saccharin, or a chemically related derivative thereof, for use in increasing susceptibility of bacteria to antibiotic treatment.

The bacteria may be resistant to the antibiotic, and the composition may render the bacteria sensitive to the antibiotic.

The composition may be for co-administration with or may include an antibiotic. The antibiotic may be a beta-lactam antibiotic, a carbapenem antibiotic, an aminoglycoside antibiotic, or a polymyxin.

The active agent may be present below a minimum inhibitory concentration. For example, it may be present at less than 15% (w/v), less than 10% (w/v), less than 5% (w/v), less than 3% (w/v), at approximately 1% (w/v), or less than 1% (w/v).

The composition may be formulated for application to a patient's skin. For example, it may be in the form of a liquid, cream, ointment, gel or hydrogel, which may be incorporated into a wound dressing.

The composition may be formulated for inhalation. For example, the composition may be in aerosolised or dry powder form.

The composition may also be formulated for intravenous administration.

According to another aspect of the present invention, there is provided a method of enhancing the activity of an antibiotic, including providing a composition including ace-K, cyclamate, saccharin or a derivative thereof, and administering the composition to a patient in need thereof, for example a human or animal patient.

According to another aspect of the present invention, there is provided a method of increasing the susceptibility of bacteria to antibiotic treatment including: providing a composition including an active agent, wherein the active agent is ace-K, cyclamate, saccharin, or a chemically related derivative thereof; providing an antibiotic; and administering the composition and the antibiotic to a subject in need thereof.

Embodiments of the present invention are now described, by way of example only, with reference to the accompanying drawings, in which:

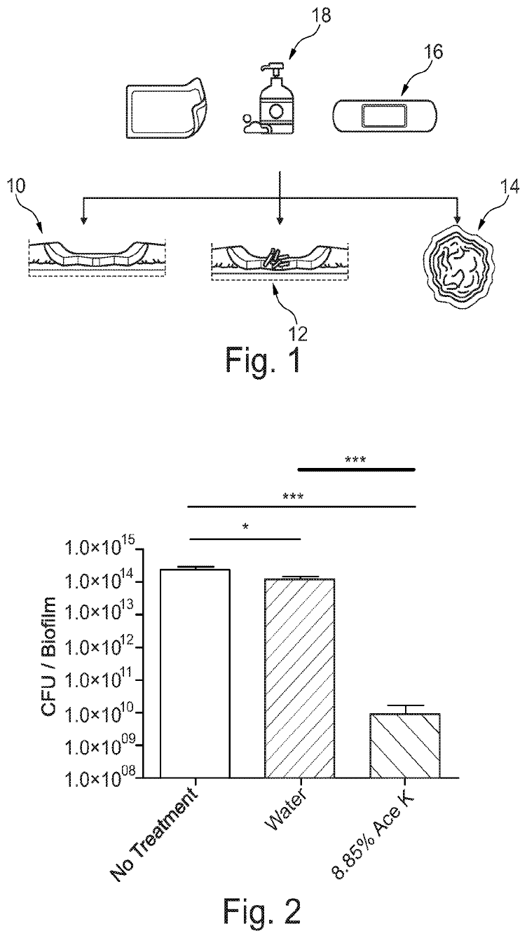

FIG. 1 schematically illustrates direct application to skin of embodiments of antibacterial compositions;

FIG. 2 shows that an ace-K wash of a P. aeruginosa colony biofilm can significantly reduce viable cell recovery;

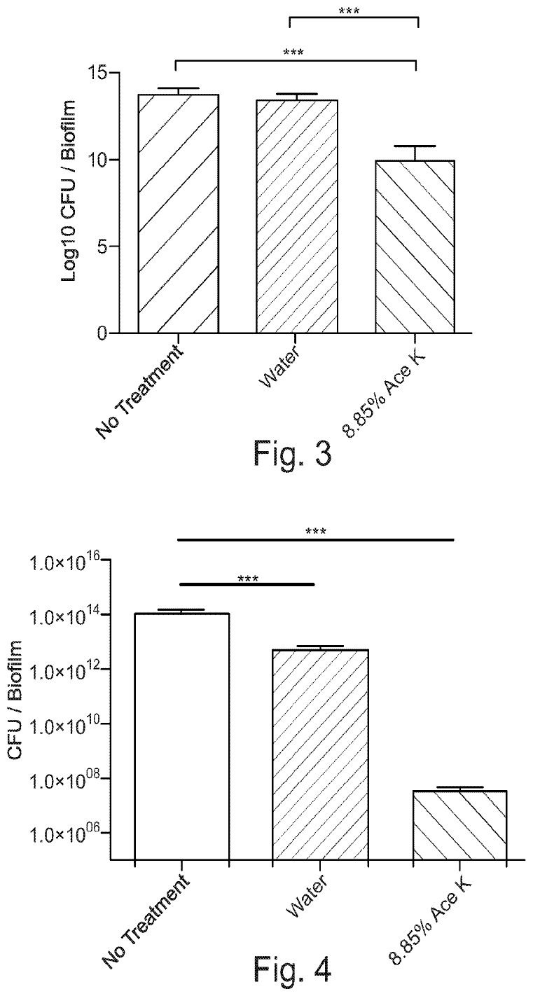

FIG. 3 shows that an ace-K wash of an A. baumannii colony biofilm can significantly reduce viable cell recovery;

FIG. 4 shows that an ace-K augmented wound dressing can significantly reduce viable cell recovery in P. aeruginosa colony biofilms;

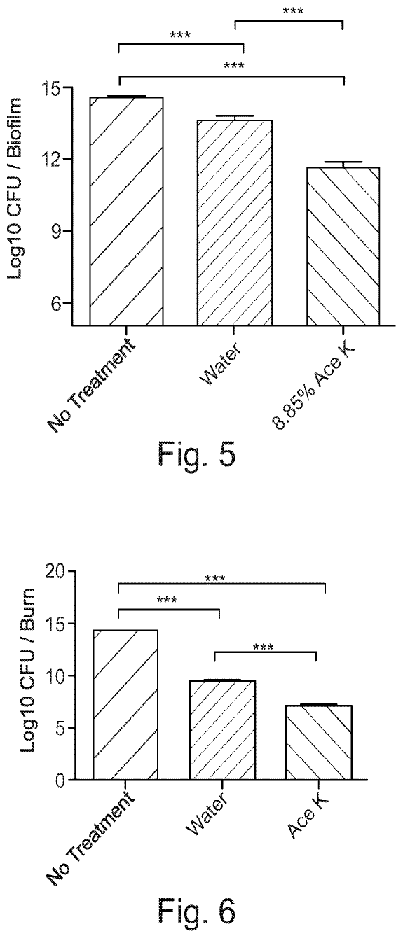

FIG. 5 shows that an ace-K augmented wound dressing can significantly reduce viable cell recovery in A. baumannii colony biofilms;

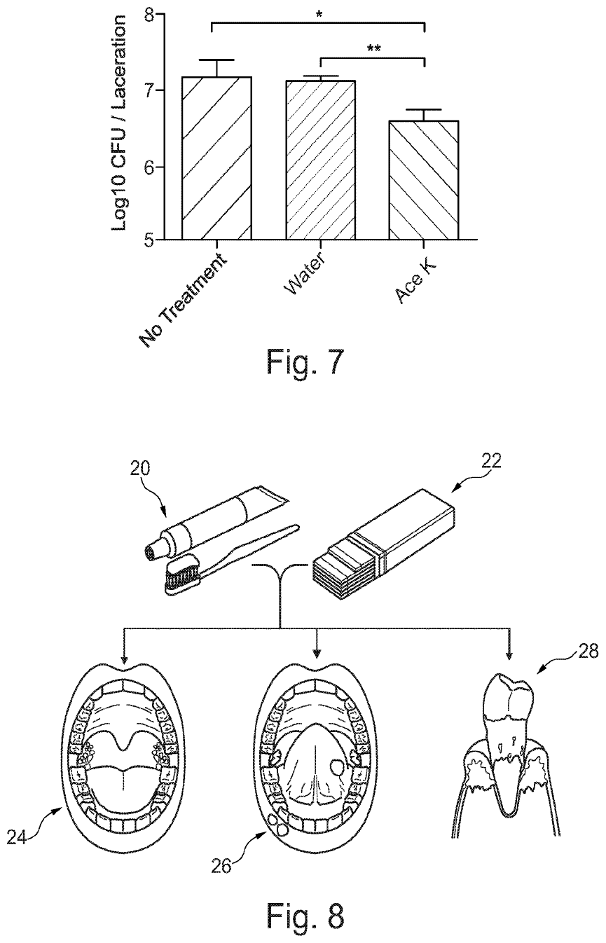

FIGS. 6 and 7 show that an ace-K augmented wound dressing can significantly reduce viable cell recovery in A. baumannii infected burn wound or laceration in a porcine skin explant model;

FIG. 8 schematically illustrates oral administration of embodiments of antibacterial compositions;

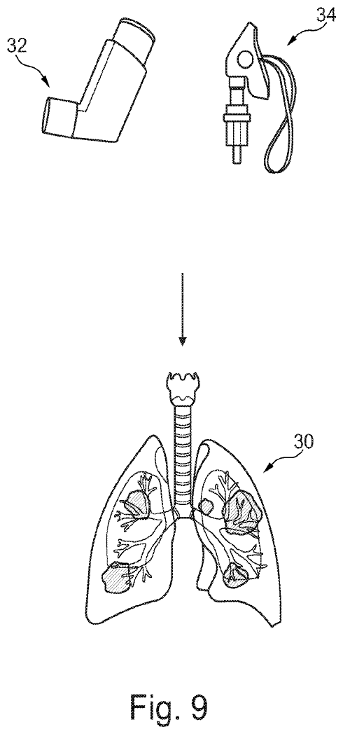

FIG. 9 schematically illustrates administration by inhalation of embodiments of antibacterial compositions;

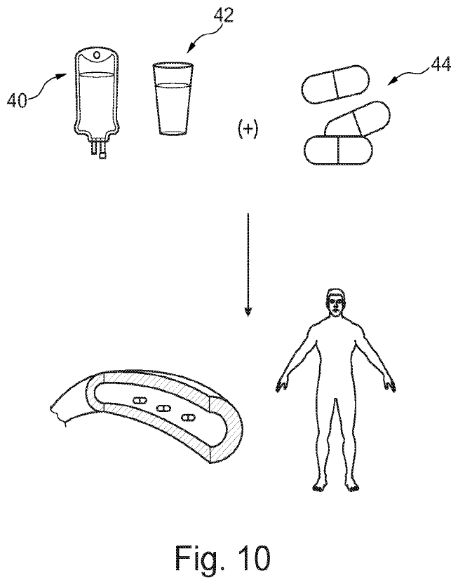

FIG. 10 schematically illustrates intravenous administration of embodiments of antibacterial compositions;

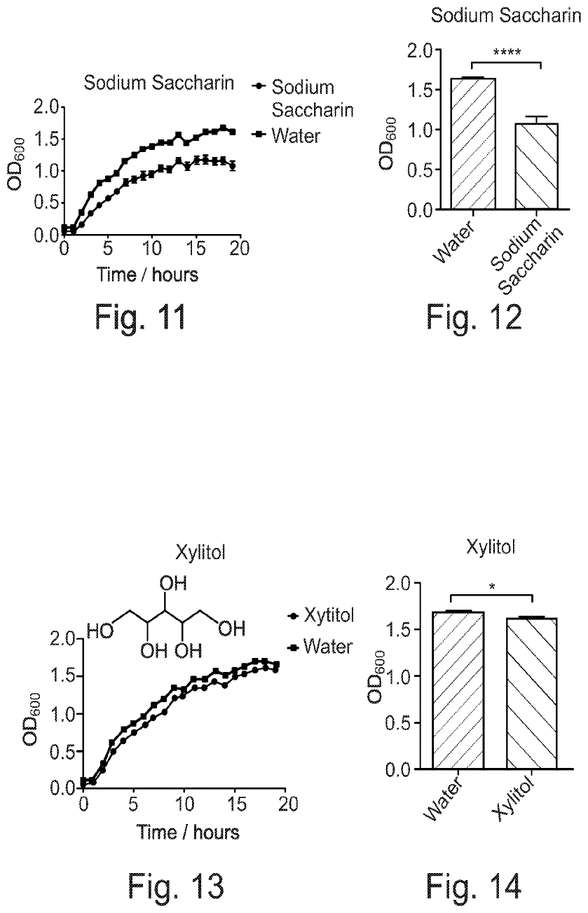

FIGS. 11 and 12 show P. aeruginosa growth in the presence of saccharin;

FIGS. 13 and 14 show P. aeruginosa growth in the presence of xylitol;

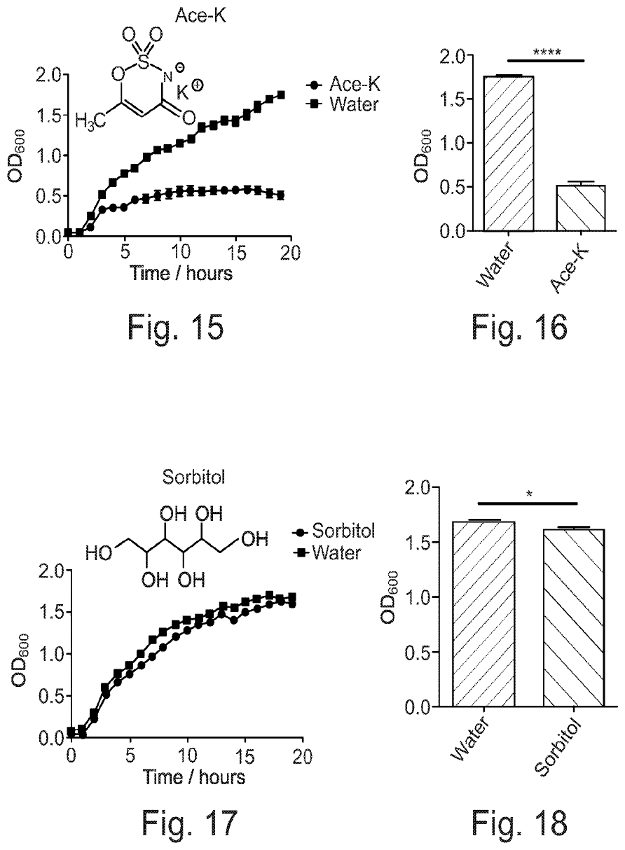

FIGS. 15 and 16 show P. aeruginosa growth in the presence of ace-K;

FIGS. 17 and 18 show P. aeruginosa growth in the presence of sorbitol;

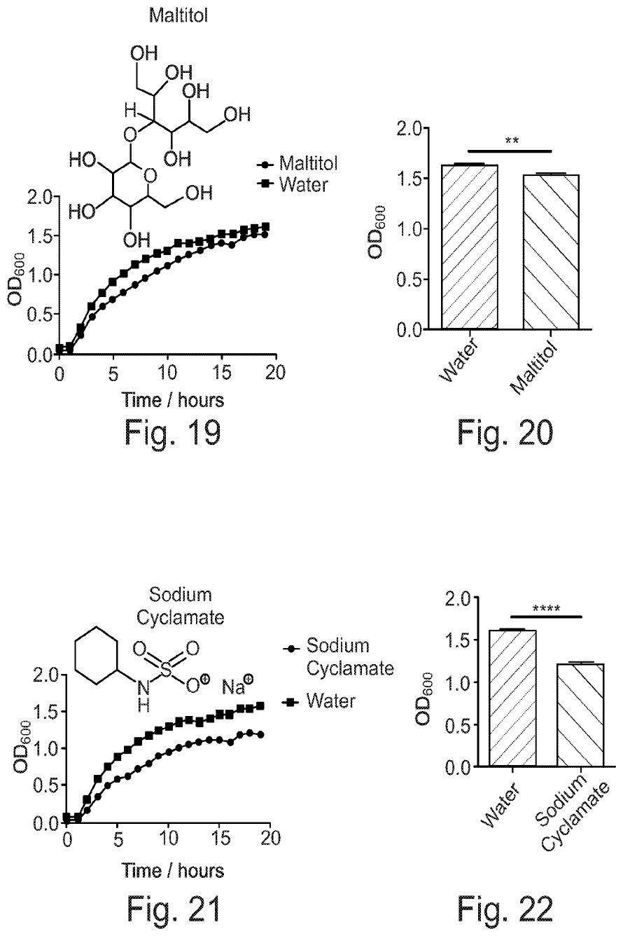

FIGS. 19 and 20 show P. aeruginosa growth in the presence of maltitol;

FIGS. 21 and 22 show P. aeruginosa growth in the presence of cyclamate;

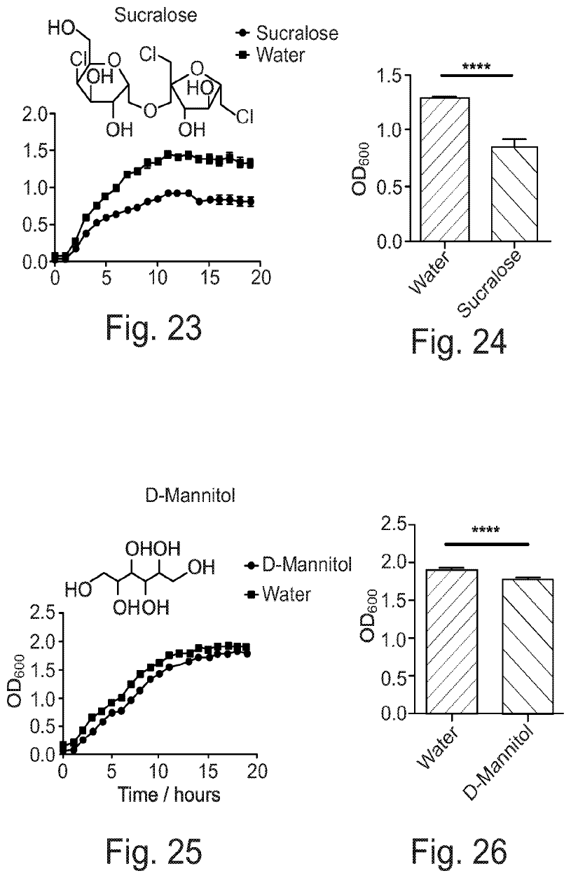

FIGS. 23 and 24 show P. aeruginosa growth in the presence of sucralose;

FIGS. 25 and 26 show inhibition of growth of A. baumannii in the presence of D-mannitol;

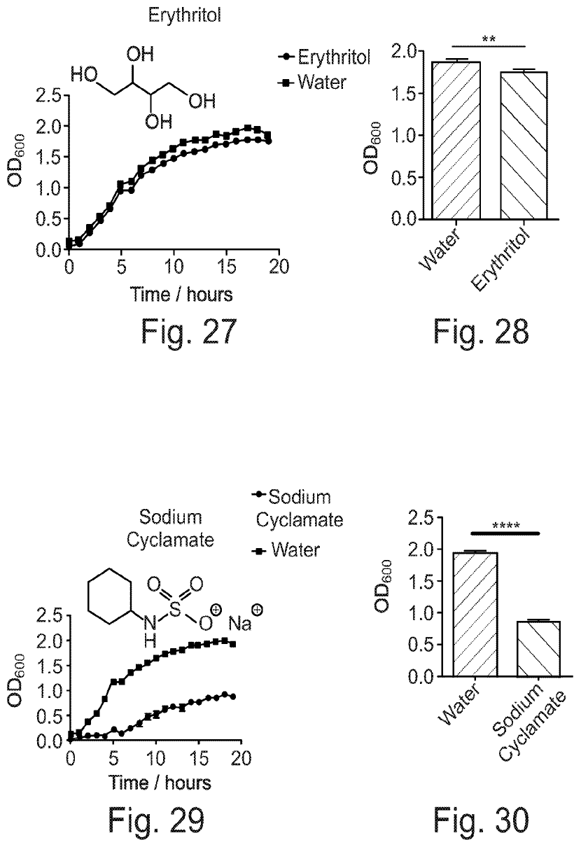

FIGS. 27 and 28 show inhibition of growth of A. baumannii in the presence of erythritol;

FIGS. 29 and 30 show inhibition of growth of A. baumannii in the presence of sodium cyclamate;

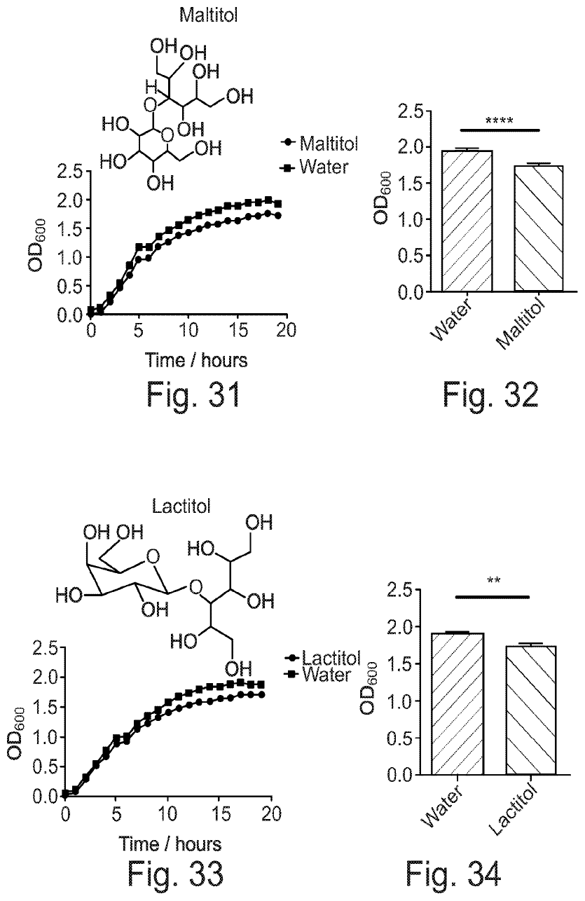

FIGS. 31 and 32 show inhibition of growth of A. baumannii in the presence of maltitol;

FIGS. 33 and 34 show inhibition of growth of A. baumannii in the presence of lactitol;

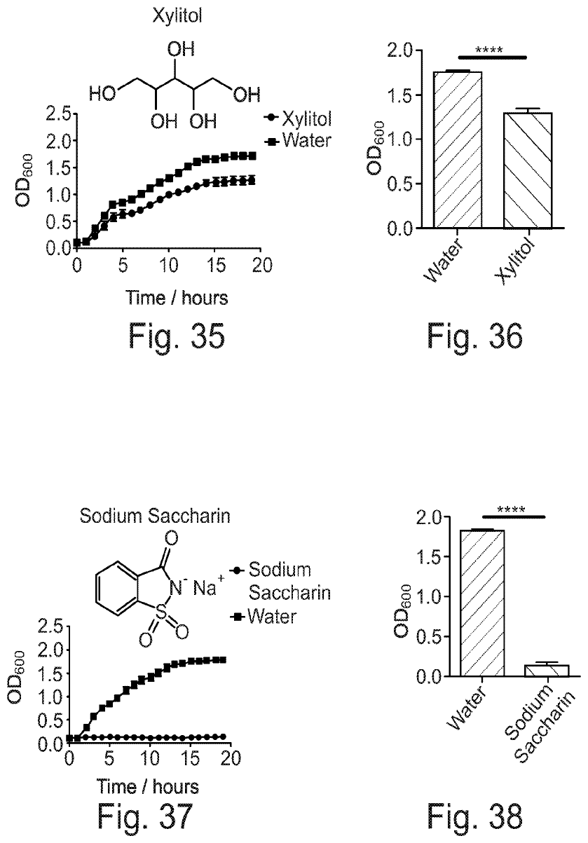

FIGS. 35 and 36 show inhibition of growth of A. baumannii in the presence of xylitol;

FIGS. 37 and 38 show inhibition of growth of A. baumannii in the presence of saccharin;

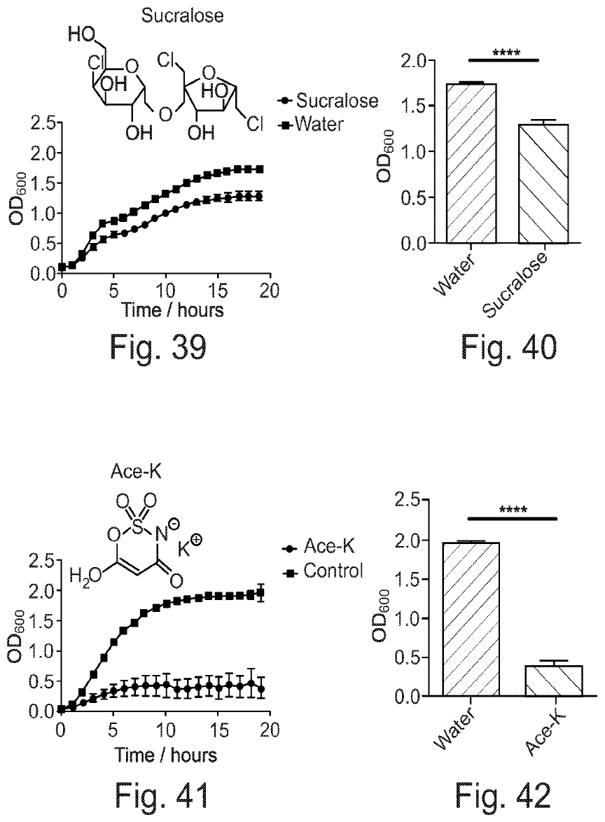

FIGS. 39 and 40 show inhibition of growth of A. baumannii in the presence of sucralose;

FIGS. 41 and 42 show inhibition of growth of A. baumannii in the presence of ace-K;

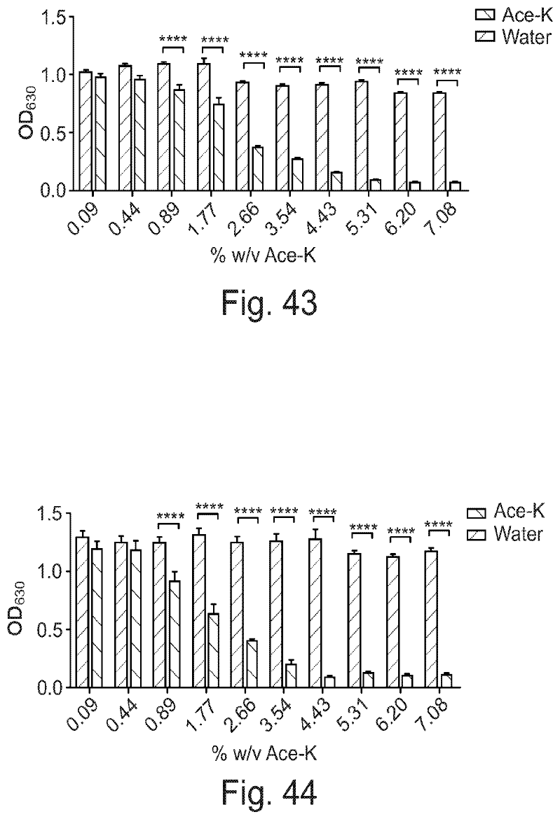

FIG. 43 shows growth of P. aeruginosa in different concentrations of ace-K;

FIG. 44 shows growth of A. baumannii in different concentrations of ace-K;

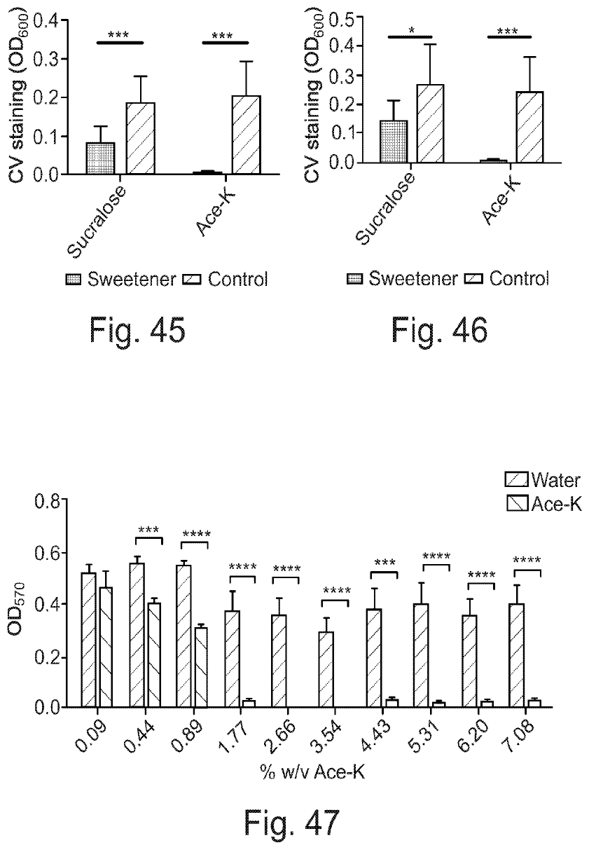

FIG. 45 shows the ability of P. aeruginosa to form biofilm after 19 hour exposure to sweeteners in LB medium;

FIG. 46 shows the ability of A. baumannii to form biofilm after 19 hour exposure to sweeteners in LB medium;

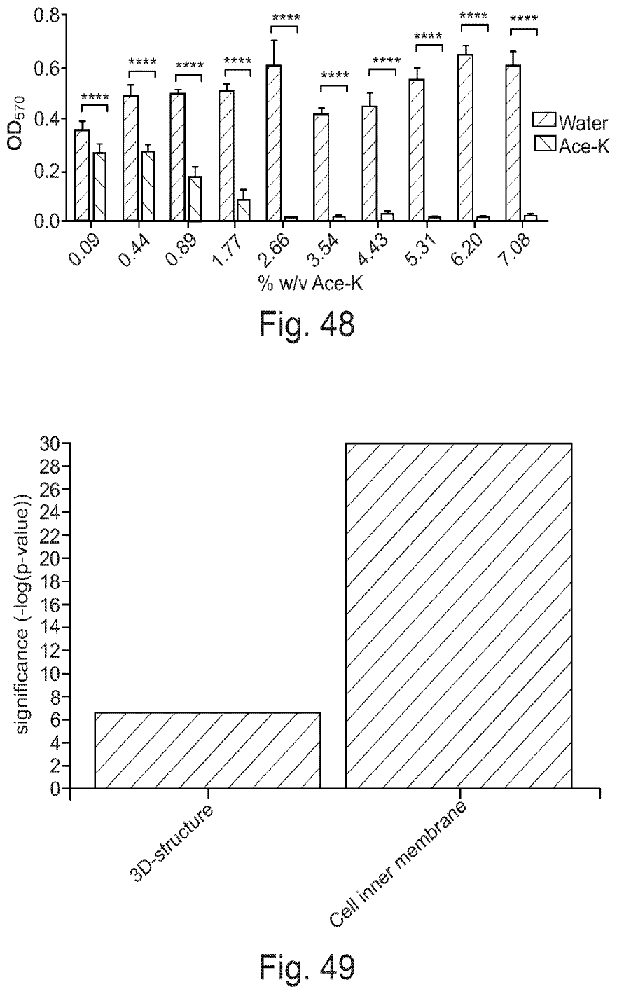

FIGS. 47 and 48 show the minimum biofilm inhibition concentrations for P. aeruginosa and A. baumannii respectively;

FIG. 49 shows the key words overrepresented in a gene set enrichment analysis;

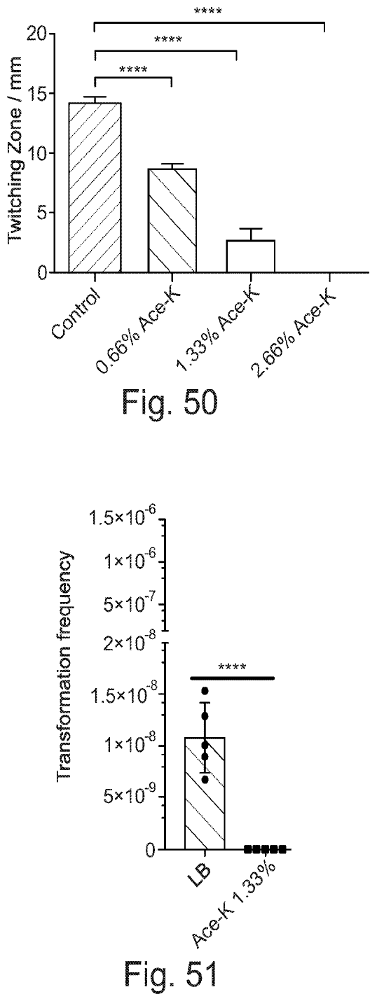

FIG. 50 shows the impact of ace-K on bacterial motility;

FIG. 51 shows the impact of ace-K on natural transformation;

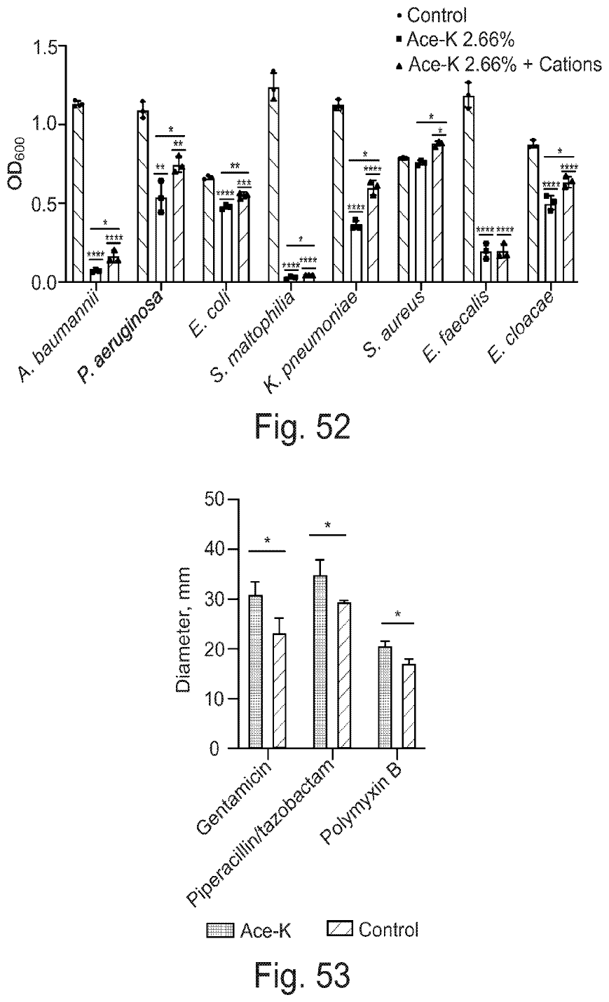

FIG. 52 shows that cation supplementation can mitigate the growth inhibition effect of ace-K;

FIG. 53 shows antibiotic sensitivity of P. aeruginosa in the presence of ace-K;

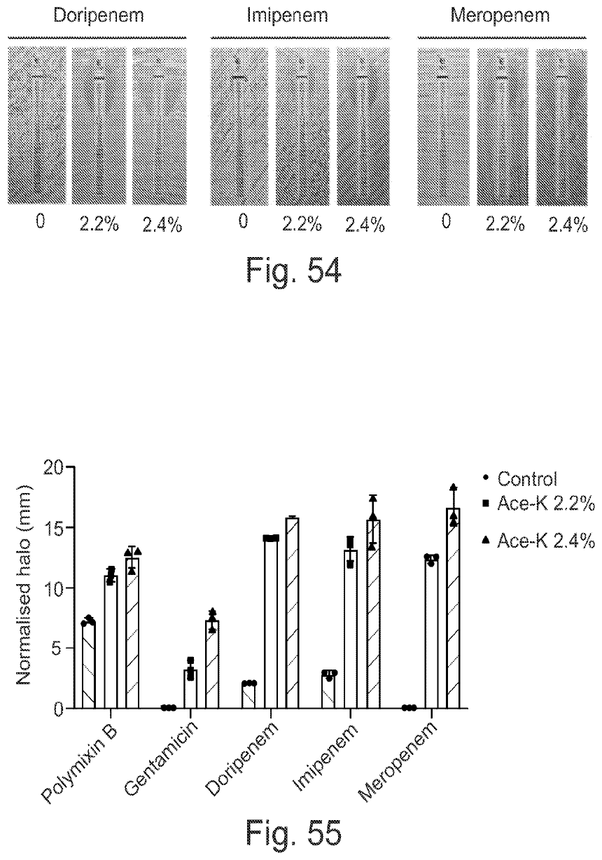

FIGS. 54 and 55 show antibiotic sensitivity of A. baumannii in the presence of ace-K;

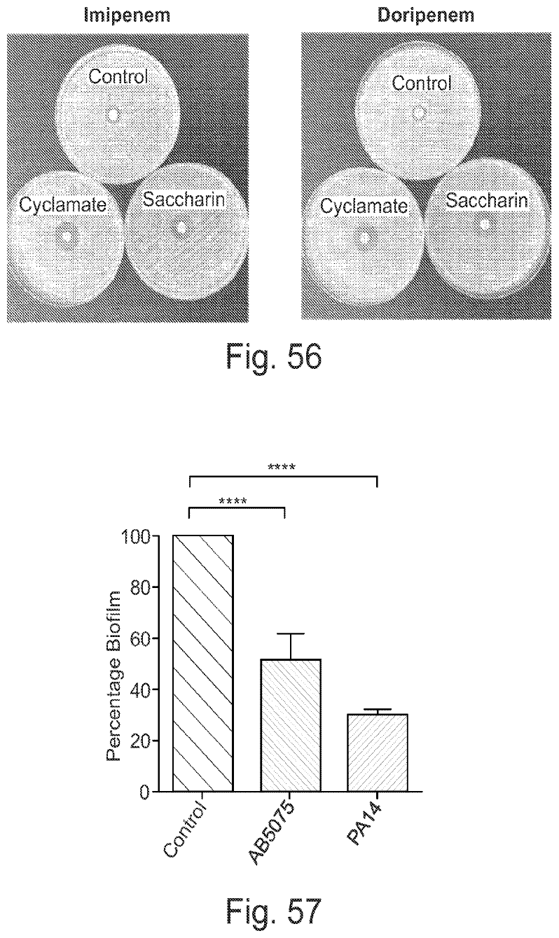

FIG. 56 shows antibiotic sensitivity of A. baumannii in the presence of cyclamate and saccharin;

FIG. 57 shows the ability of ace-K to disperse an established biofilm;

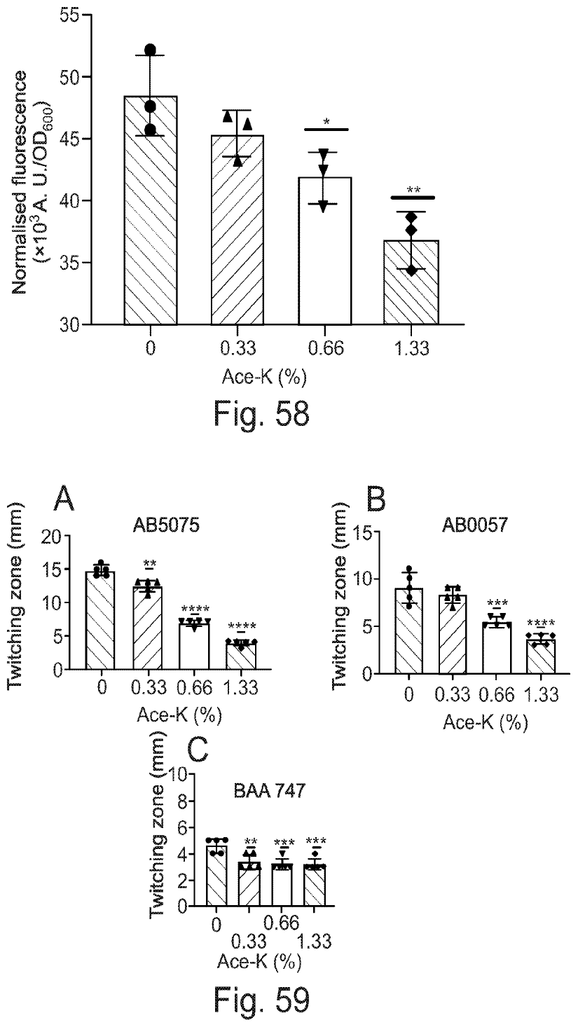

FIG. 58 shows the ace-K dose dependent reduced expression of the pilA promoter;

FIG. 59 shows the dose dependent inhibition of twitching motility in multiple strains of A. baumannii;

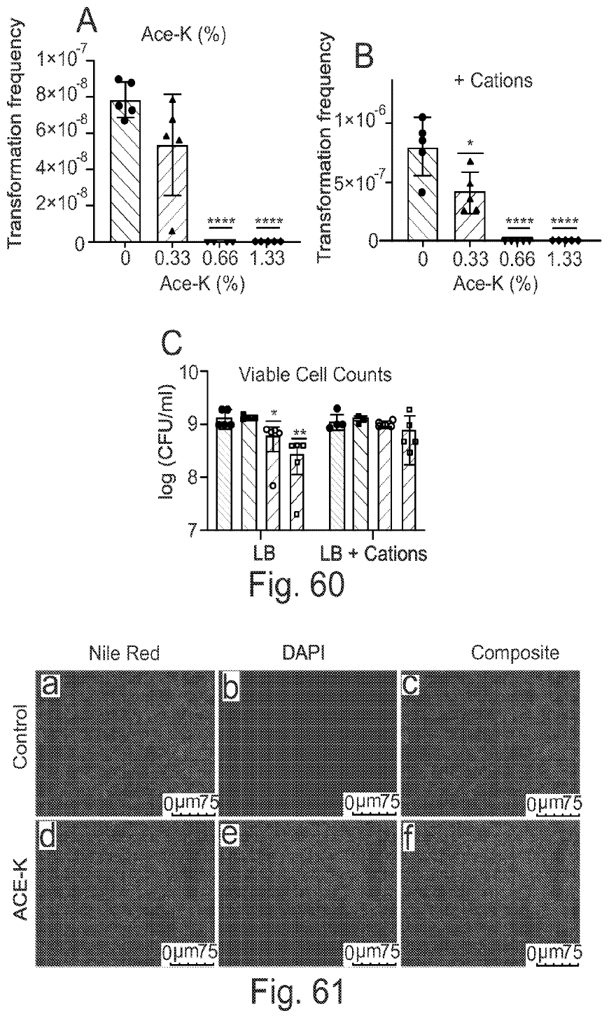

FIG. 60 demonstrates the dose dependent impact of ace-K on natural transformation;

FIG. 61 shows the impact of ace-K on membrane permeability;

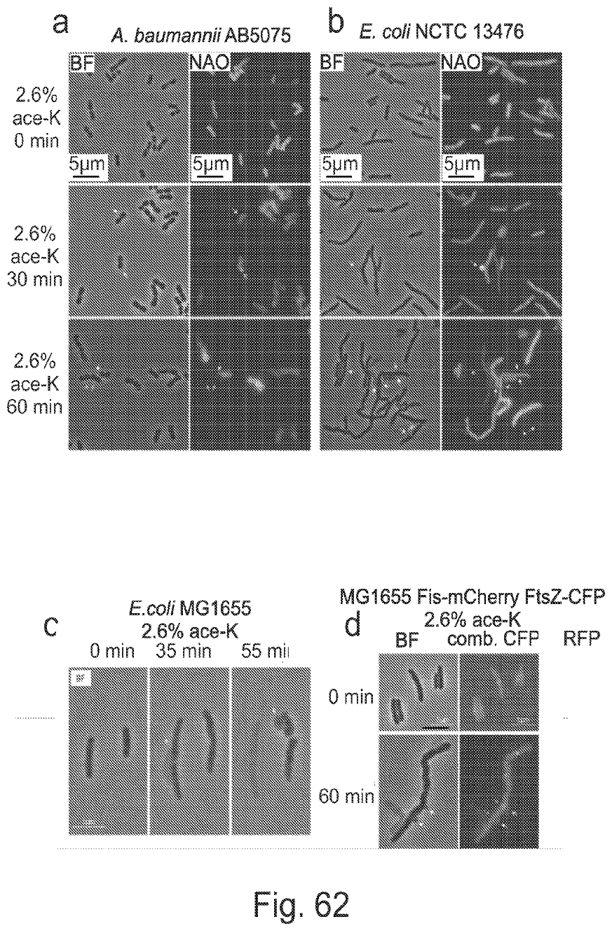

FIG. 62 demonstrates the impact of ace-K on cell morphology, the induction of cell envelope bulges and cell lysis;

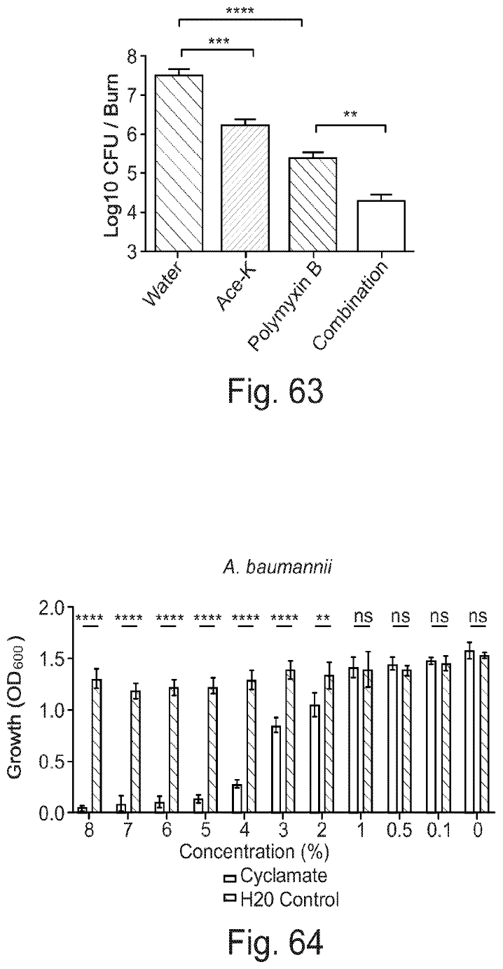

FIG. 63 demonstrates the antibiotic potentiating effect of ace-K in an ex vivo wound model;

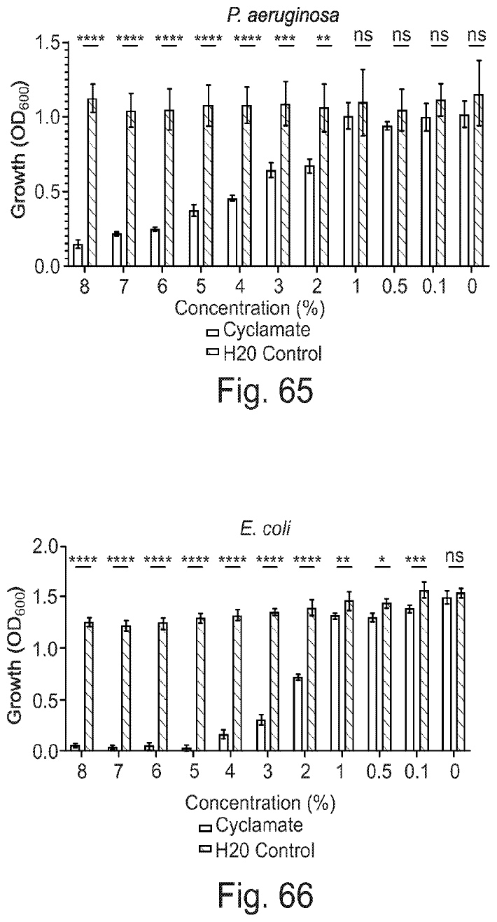

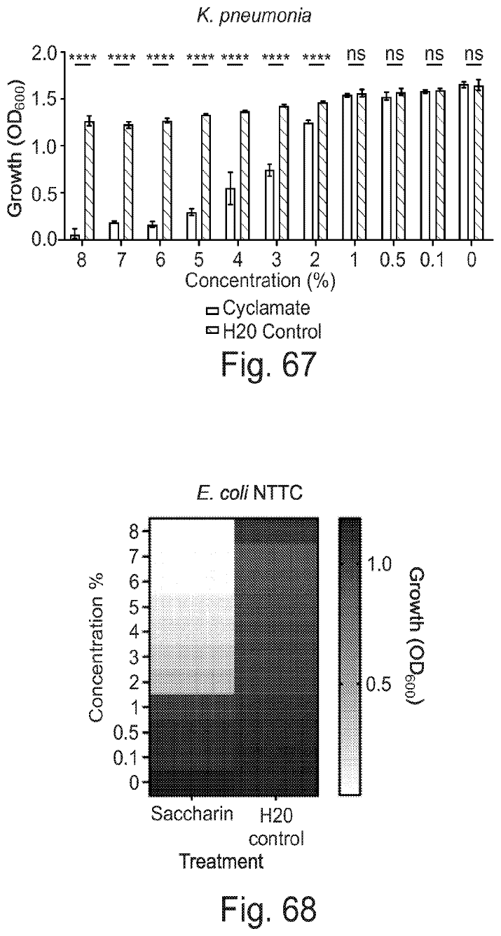

FIGS. 64 to 67 show the results of growth inhibition assays for A. baumannii, P. aeruginosa, E. coli, and K. pneumonia using cyclamate;

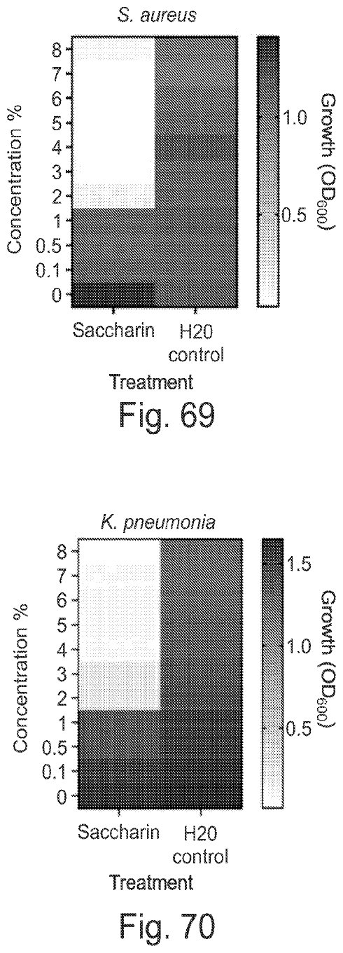

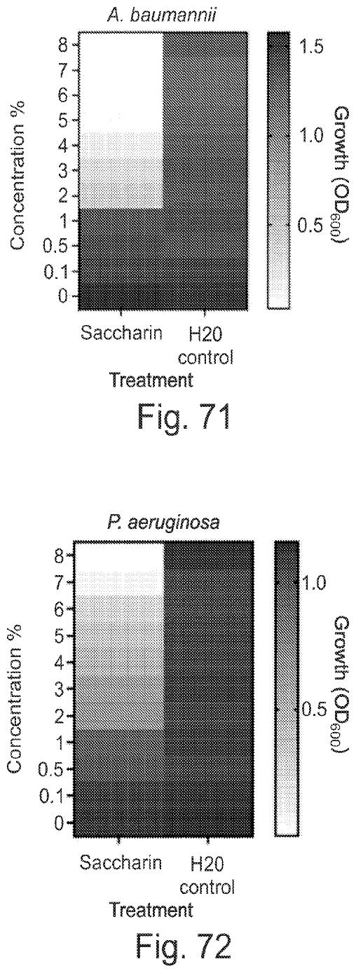

FIGS. 68 to 72 show the results of growth inhibition assays for E. coli, S. aureus, K. pneumonia, A. baumannii, and P. aeruginosa using saccharin;

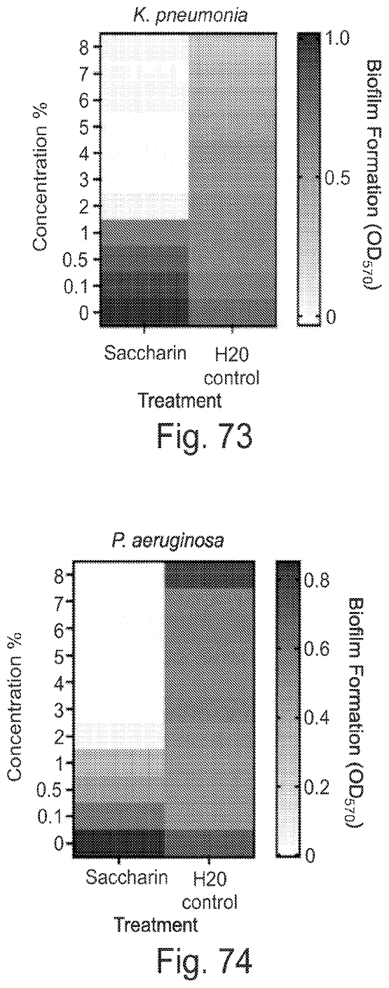

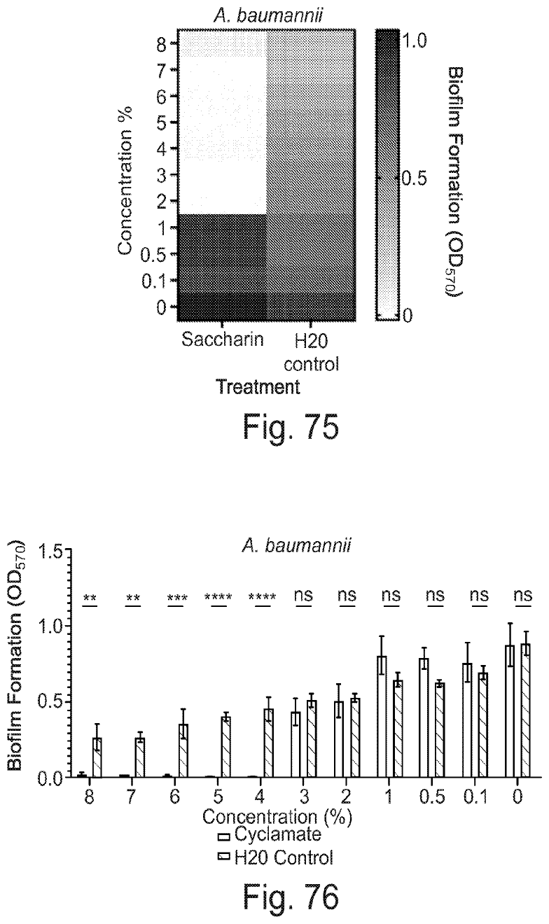

FIGS. 73 to 75 show that saccharin inhibits biofilm formation in K. pneumonia, P. aeruginosa, and A. baumannii;

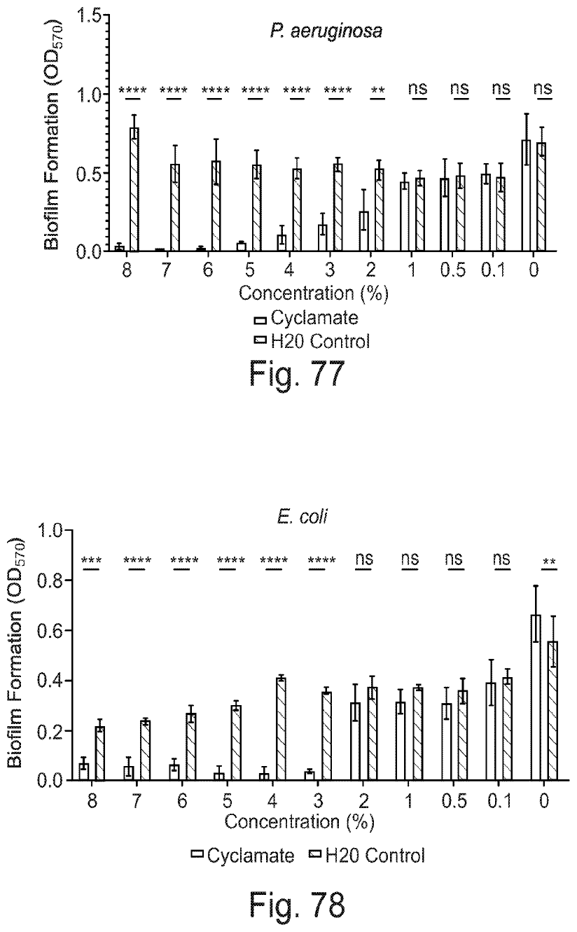

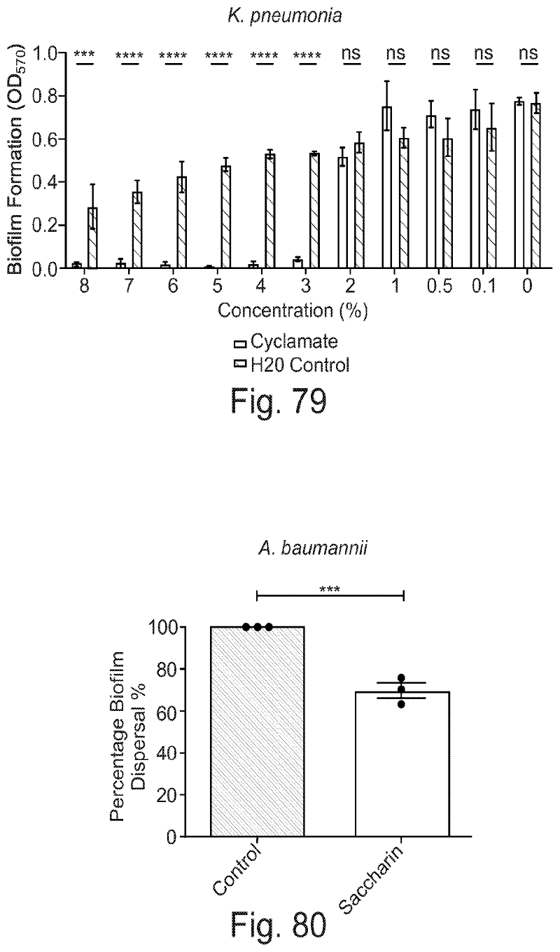

FIGS. 76 to 79 show the results of cyclamate inhibition of biofilm formation in A. baumannii, P. aeruginosa, E. coli, and K. pneumonia;

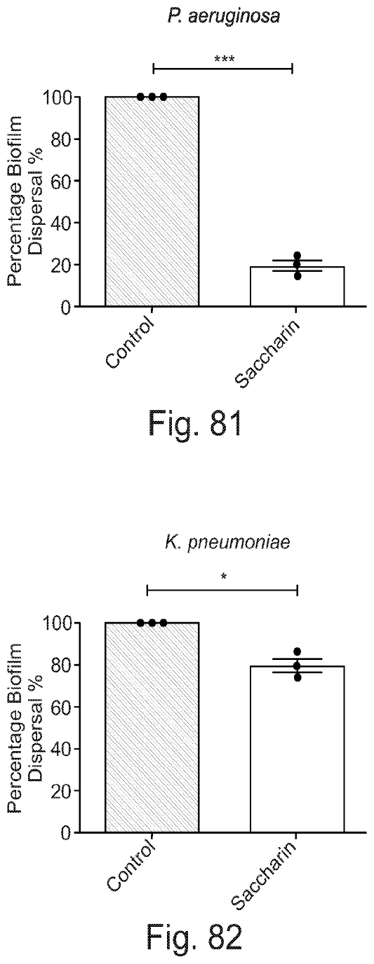

FIGS. 80, 81 and 82 show bacterial biofilm dispersal results;

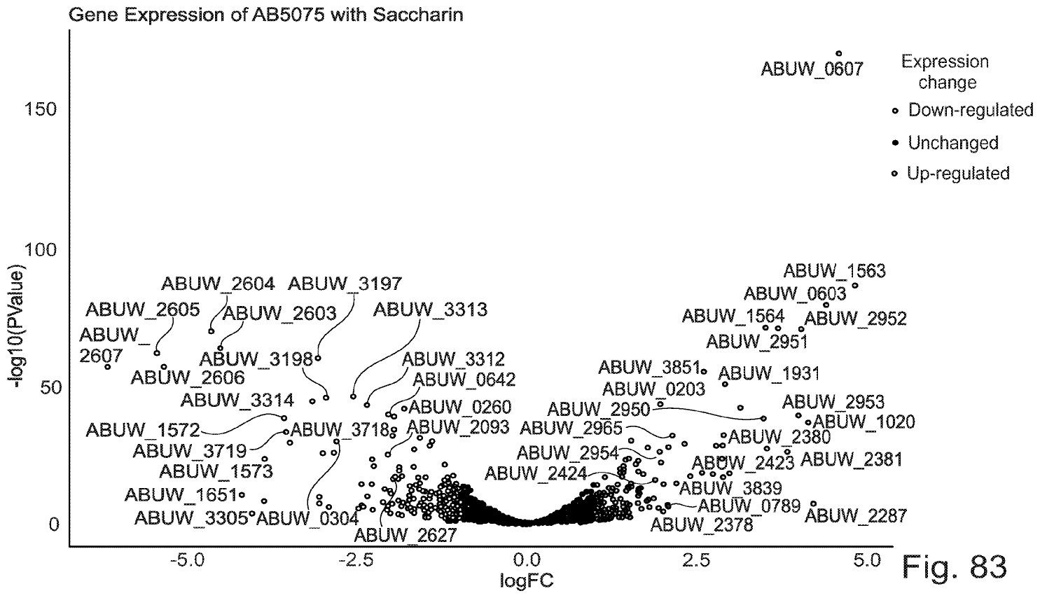

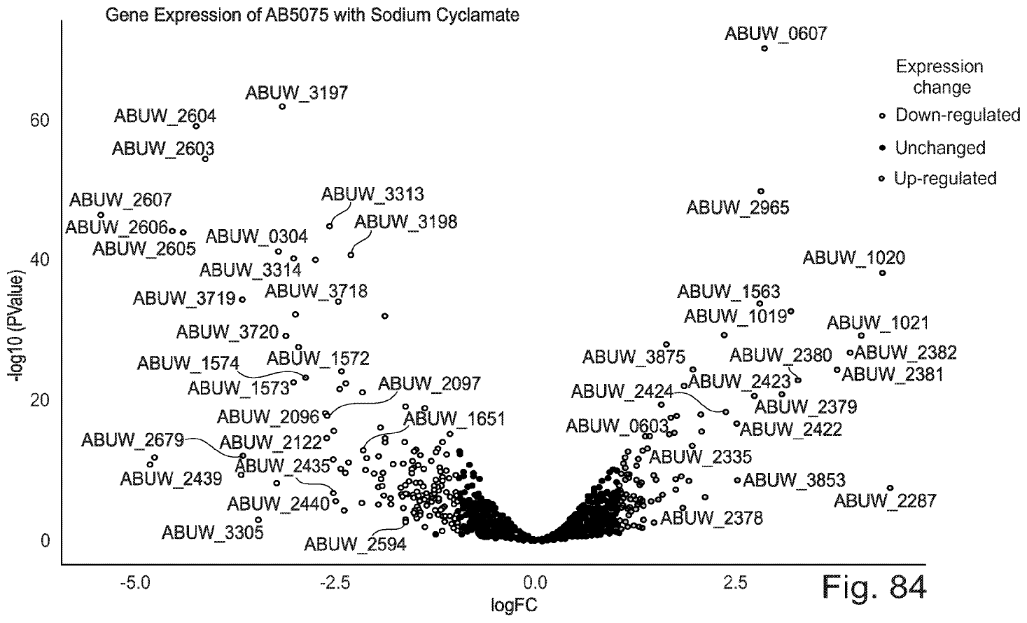

FIGS. 83 and 84 show the impact of saccharin and cyclamate on bacterial gene expression;

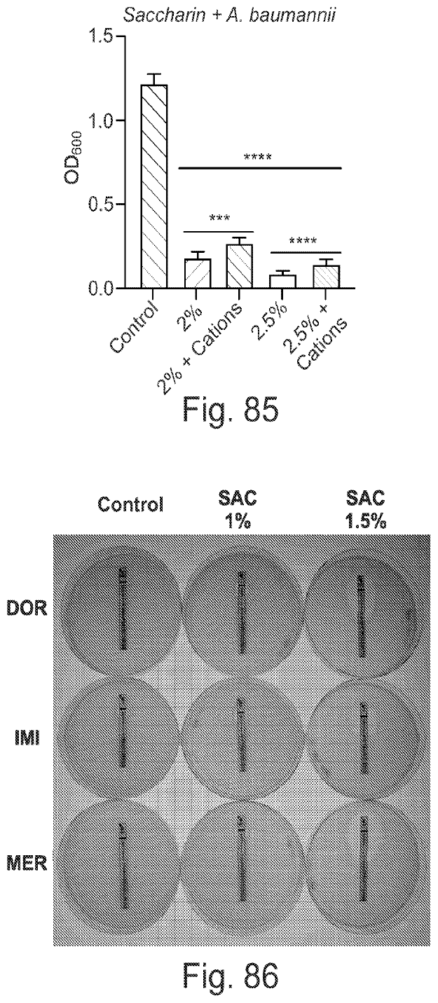

FIG. 85 shows the effect of cations on growth of A. baumannii in the presence of saccharin;

FIG. 86 demonstrates the ability of saccharin to increase the sensitivity of A. baumannii to carbapenems;

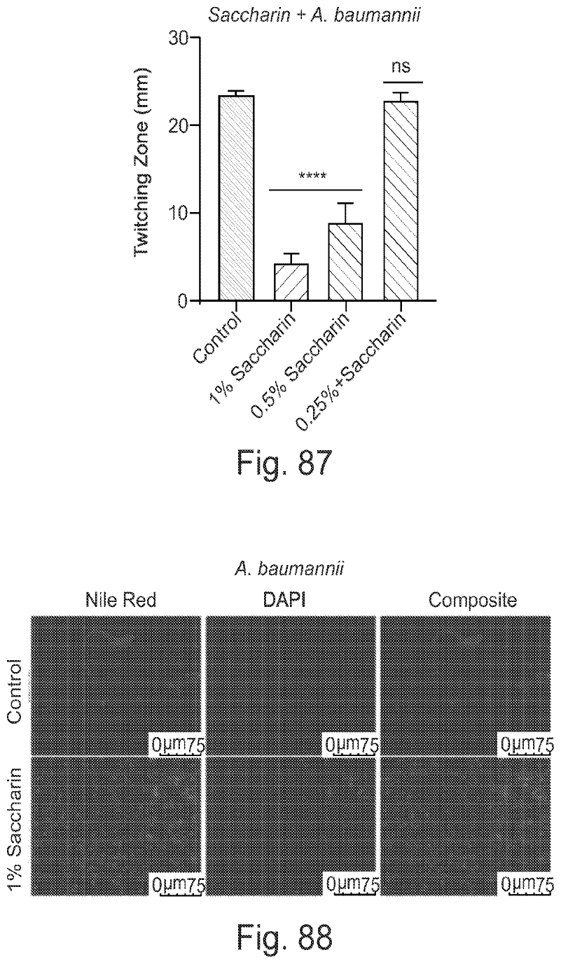

FIG. 87 shows a dose-dependent decrease in the twitching motility of A. baumannii over increasing concentrations of saccharin;

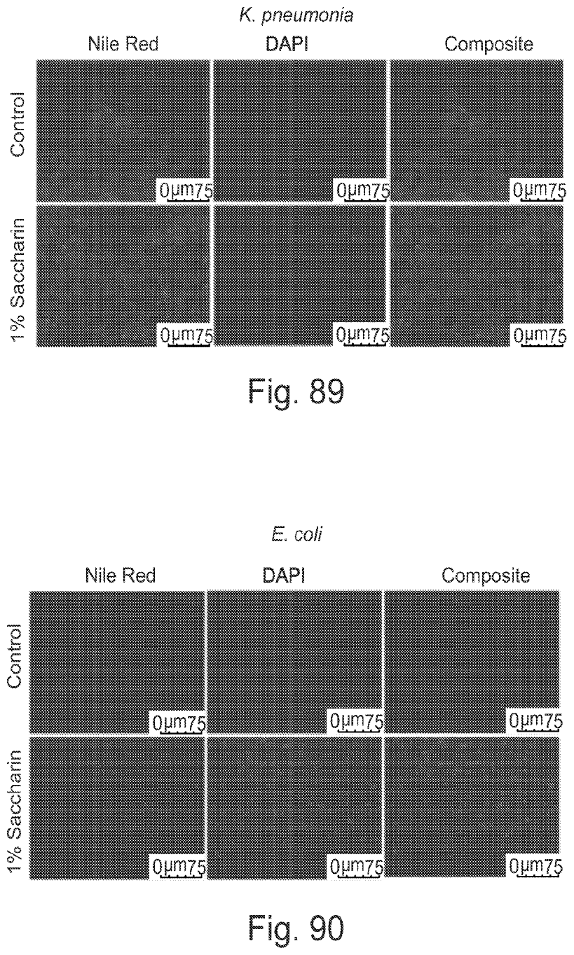

FIGS. 88-90 show increased staining with DAPI of A. baumannii, K. pneumoniae, and E. coli;

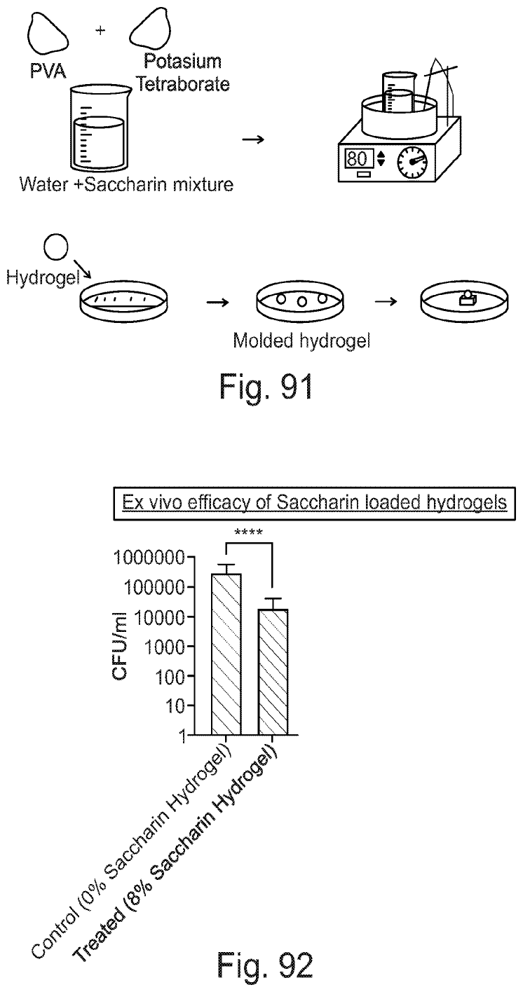

FIG. 91 schematically illustrates a method of preparing a hydrogel loaded with a sweetener; and

FIG. 92 shows the therapeutic potential of saccharin hydrogel in a porcine ex vivo burn wound model.

The global increase in obesity due to excessive sugar consumption has propelled the discovery and inclusion of many artificial sweeteners into diets. These artificial sweeteners are FDA approved and deemed safe to consume at relatively high concentrations. There are many known artificial sweeteners. They are chemically diverse, though some (such as the sugar alcohols) are chemically related.

Ace-K is 200 times sweeter than sucrose. It is the potassium salt of 6-methyl-1,2,3-oxathiazine-4 (3H)-one 2,2-dioxide:

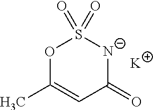

Xylitol is a sugar alcohol having a similar sweetness to sucrose:

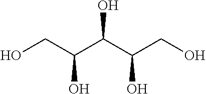

Mannitol is also a sugar alcohol, and is about 50% as sweet as sucrose:

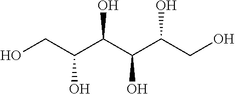 [SMILES: OC[C@@H](O)[C@@H](O)[C@H](O)[C@H](O)CO]

Erythritol is also a sugar alcohol, and is 60-70% as sweet as sucrose:

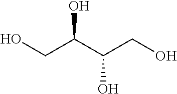

Maltitol is also a sugar alcohol, having 75-90% the sweetness of sucrose:

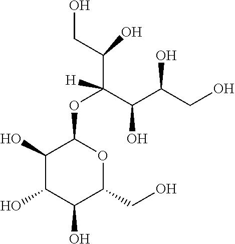 [SMILES: OC[C@@H](O)[C@@H](O[C@H]1O[C@H](CO)[C@@H](O)[C@H](O)[C@H]1O)[C@H](O)[C@@H](O)CO]

Lactitol is also a sugar alcohol, and has 30-40% the sweetness of sucrose:

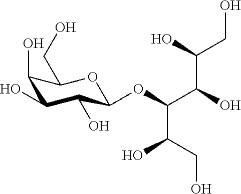

Sucralose can be 320-1000 times as sweet as sucrose. It is a derivative of sucrose containing chlorine groups:

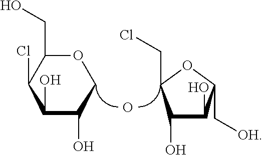

Cyclamate is 30-50 times sweeter than sucrose. It is the sodium or calcium salt of cyclamic acid. By way of example, the formula for the sodium salt is given below:

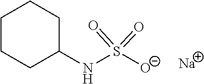 [SMILES: O=S(=O)([O-])NC1CCCCC1 ; [Na+]]

Saccharin is >500 times sweeter than sucrose. It is usually used in foods in its sodium or calcium salt form:

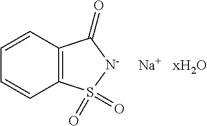 [SMILES: O=C1[N-]S(=O)(=O)c2ccccc21 ; [Na+]]

There are many other artificial sweeteners that have been approved for use in the human diet such as sorbitol, D-tagatose, aspartame-acesulfame salt, 1′,4,6′-trichlorogalactosucrose, glycyrrhizin, neotame, aspartame, advantame, salt of aspartame acesulfame, thaumatin and hydrogenated starch hydrolysates.

Recent research has been exploring the effect that artificial sweeteners have on healthy bacteria in the gut but the findings are controversial. Some studies demonstrate that the growth of gut bacteria is induced in the presence of sweeteners, while others present the opposite.

WO 2014/082050 discloses use of sweeteners as an excipient in antibacterial compositions containing antibacterial agents. However, there is no disclosure of the sweeteners themselves having any antibacterial effect.

With respect to the published literature, as with the effect of artificial sweeteners on human health, there is conflicting data available on the impact of artificial sweeteners on bacterial growth, for example Shahriar et al. (2020) show that acesulfame potassium (ace-K) promotes bacterial growth, as do Mahmud et al. (2019). Contrary to this positive growth effect, there is one study that mentions a negative impact on growth of laboratory Escherichia coli strains (Wang et al. (2018)). The impact of artificial sweeteners on the gut microbiome has also been explored (Bian et al. (2017), Wang et al. (2018)). Sucralose has been shown to inhibit motility in the pathogen P. aeruginosa via quorum sensing inhibition (Markus et al. (2021)).

Two recently published studies have indicated that some artificial sweeteners, including ace-k, can promote the dissemination of antibiotic resistance genes through horizontal transfer either by natural transformation or conjugative gene transfer (Yu et al. (2021a); Yu et al. (2021b)). However these studies were not performed in pathogens.

As can be seen from the above discussion, studies to date on the effect of artificial sweeteners on bacterial growth have been somewhat inconsistent. To date, no-one has studied artificial sweeteners as antibacterials, in particular in regard to having activity against known pathogenic bacteria.

The present applicant investigated the effect of several common artificial sweeteners on growth of a range of clinically relevant pathogens, and also studied their effect of a range of different virulence associated behaviours. Furthermore, the effect of these sweeteners on the efficacy of a range of commonly used antibiotics was investigated.

From its results, several artificial sweeteners with antimicrobial properties were identified. In particular, the artificial sweeteners displayed robust anti-bacterial activity against four of the six most commonly antibiotic resistant bacterial pathogens (A. baumannii, P. aeruginosa, Staphylococcus aureus, Klebsiella pneumoniae, and Enterobacter species). These pathogens are the major cause of nosocomial infections and can persist even after being treated with antimicrobial agents.

The applicant has demonstrated that as well as inhibiting growth, ace-K in particular is capable of inhibiting a range of virulence behaviours such as biofilm formation (associated with persistent infection) and motility (associated with dissemination throughout the body). Remarkably, at least cyclamate, saccharin and ace-K can also potentiate the activity of a range of clinically relevant antibiotics. The applicant also uncovered the mechanism of this activity using RNA sequencing.

The applicant proposes the use of artificial sweeteners to treat or prevent infection. Specifically, it has demonstrated that artificial sweeteners will inhibit bacterial growth and disrupt bacterial behaviours associated with virulence, including chronic infection phenotypes such as biofilm formation. It has also been shown that these artificial sweeteners can potentiate the activity of a range of clinically relevant antibiotics on bacteria that are, without presence of the sweeteners, generally resistant to those antibiotics. It is proposed that these compounds can be used to treat or prevent infection.

Particularly preferred artificial sweeteners for use in the compositions and methods disclosed herein are those having a sulphonamide group, for example, ace-K, cyclamate and saccharin. These were found to be particularly potent at inhibiting the growth of multidrug resistant pathogens.

It is envisaged that these compounds could be used to treat or prevent infections in several ways, for example topical application in the form of a liquid, cream, ointment, gel or wound dressing, oral administration, aerosolised or dry powder administration (for inhalation), or intravenous administration. In all applications, the artificial sweetener could be applied alone, or in combination with a prescribed antibiotic regime, to potentiate antibiotic activity where the organism would otherwise be resistant to the antibiotic. It is proposed that each of these applications could be used prophylactically to prevent infection or actively to treat infection. It is also proposed that each of these applications could be used in combination with antibiotic therapy to potentiate antimicrobial activity of the antibiotic.

Given the current antibiotic crisis and its impact on the health care sector, there is an urgent need for novel antimicrobial treatments that can be deployed rapidly and, in some instances, deployed prophylactically to limit the overuse of antibiotic therapies.

The applicant has identified a range of artificial sweeteners that can significantly impact the growth of a range of the most prevalent and problematic multidrug resistant pathogens. These compounds could be prescribed by a medical practitioner as a treatment or a prophylactic. However, given the favourable status that these artificial sweeteners have with global food and drug authorities it is possible that they may be developed as an over the counter treatment or supplement.

The antimicrobial activity of artificial sweeteners holds significant clinical potential. Particular advantages in using these artificial sweeteners as antibacterial compounds include:

Broad spectrum effect against multidrug resistant pathogens.

Much of pharmacokinetics and pharmacodynamics already known.
Favourable status with food and drug administrations.
Already part of the diet of many individuals.
Multi-impacts on the cell.
They can disrupt established biofilms, one of the leading causes of routine antibiotic failure.
Mechanism of action understood.
Can increase sensitivity of resistant pathogens to common antibiotics.
Can increase sensitivity to carbapenems (carbapenem resistant pathogens being a major threat to health).

The skilled person will appreciate that the artificial sweeteners exemplified below could be used alone, or together in different combinations. They would also understand that pharmaceutically acceptable salts of the sweeteners, and chemically related derivatives of the exemplified compounds can also be used to achieve the same effects. Some embodiments can involve adding antibacterial artificial sweeteners to existing anti-infective or antimicrobial formulations.

### EXAMPLES

### Example 1: Direct Application

In a first example, schematically illustrated in FIG. 1, an artificial sweetener can be applied directly to an acute wound that is uninfected 10, an infected acute wound 12, or to a chronic wound 14.

The artificial sweetener can be prepared in a desired solvent typically to saturation (for example, 13.5 grams of ace-K in 50 ml of sterile deionised water). The preparation is then filter sterilised through a 0.2 μm filter. This working stock solution can then be used in the preparation of all subsequent downstream applications (for example, a liquid, cream, ointment, gel, hydrogel or wound dressing).

By way of example a wound wash 18, taking the form of a prepared solution of artificial sweetener is used to flood the wound bed continuously over a defined time period. This area is then rinsed with sterile saline or water, and subsequently covered with a traditional dressing or plaster.

To explore the clinical potential of artificial sweeteners, a wash of a chronically infected wound was simulated, in order to test the effect of an ace-K wash on bacteria viability. P. aeruginosa and A. baumannii colony biofilms, representing chronically infected wounds, were submerged in a 8.85% (w/v) ace-K solution for 1 hour before resuspension, serial dilution and enumeration.

This wash treatment led to a significant reduction in the number of viable bacteria within the biofilm for both P. aeruginosa biofilms (FIG. 2) and A. baumannii biofilms (FIG. 3). Data shown is average of three biological replicates with SD. Data analysis by students t test. *p≤0.05, **p≤0.01***p≤0.001 versus the LB control.

In another example, a gauze dressing 16 is soaked in a 10% (w/v) solution of the sweetener until saturated. This dressing can then be applied to an infected wound 12 for a defined period of time to promote disinfection of the wound.

The effect of ace-K loaded gauze dressing on viability of bacteria was tested. The impact of a wound dressing augmented with ace-K on chronic wound colonisation was studied. P. aeruginosa and A. baumannii colony biofilms, representing chronically infected wounds, were covered with a surgical gauze soaked in a 8.85% ace-K solution for 1 hour before resuspension, serial dilution and enumeration.

Treatment with the augmented dressing led to significant reductions in bacteria numbers for both P. aeruginosa biofilms (FIG. 4) and A. baumannii biofilms (FIG. 5) compared to water-soaked dressing. Samples were tested in biological triplicate with technical quadruplets. Analysis was by independent t-test. *p≤0.05, **p≤0.01***p≤0.001 versus the LB control.

A similar impact was seen when these dressings were tested on a porcine ex vivo skin model. In this model, porcine skin was either burnt or lacerated and the wound infected with A. baumannii AB5075 and left for 3.5 hours to allow a biofilm to form. The dressing was applied for 1 hour and viable cells were collected after treatment. This resulted in a significant reduction in cells recovered compared to the water loaded dressing control. The results are shown in FIG. 6 for the burn model (2.16 log reduction in viable cells versus the water control), and FIG. 7 for the laceration model (0.5 log reduction in viable cells versus the water control). Samples were tested in biological triplicate with technical quadruplets. Analysis was by independent t-test *p≤0.05, *p≤0.01***p≤0.001 versus the water control.

In another example, the same dressing could be applied to an uninfected wound 10 to prevent wound colonisation by pathogens.

In another example, the sweetener may be used to load a hydrogel that can be applied to the wound 10; 12; 14 (see Example 19 below).

### Example 2: Oral Administration

The antimicrobial artificial sweeteners are commonly found in the diet, which means they could potentially be included as part of a patient's diet to limit the risk of infection or to help potentiate the effects of antibiotics in patients that have had them prescribed, from either a GP or in a hospital setting. The ADI (acceptable daily intake) of ace-K is 15 mg per kg of body weight which is equivalent to about 1000 mg for a person weighing 75 kg.

We also propose a potential mouth wash or cream could be used at even higher concentrations (for example, >0.1%) to treat oral infections.

In an example, schematically illustrated in FIG. 8, the artificial sweeteners are orally administered in the form of toothpaste 20 or chewing gum 22, for example to treat and/or prevent throat, mouth, gum and dental infections (for example, tonsillitis 24, ulcers 26, abscesses 28 and tooth decay).

Although chewing gum containing artificial sweetener is known, it has previously been used merely as a sugar substitute to provide sweetness. However, it is proposed that an artificial sweetener could be included in chewing gum at a concentration at which it disables virulence phenotypes (such as biofilm formation). This may only require a low concentration such as less than 0.1%, less than 0.4%, less than 0.44%, less than 0.5%, less than 1%, or 0.1% to 1%. At higher concentrations, (for example, greater than 0.4%, greater than 0.44%, greater than 0.5%, or greater than 1%, bacteria in the mouth could be killed by the artificial sweetener. Therefore, in the example of chewing gum, the antibacterial effect would be directly from the activity of the artificial sweetener used at anti-virulence or antibacterial concentrations and not from the indirect effects of mechanical agitation leading to bacterial removal or the indirect effect of sugar depletion in the oral microenvironment.

### Example 3: Administration by Aerosol or Dry Powder Inhalation

As schematically illustrated in FIG. 9, the artificial sweeteners (for example, saccharin, ace-K, cyclamate and derivatives thereof) could be administered to treat infections associated with lung disease 30 by inhalation of a dry powder preparation from an inhaler 32 or an aerosolised sweetener in aqueous solution from a nebuliser 34.

It is proposed that the effective artificial sweeteners could be aerosolised from a stock solution (10% w/v) using a nebuliser or inhaled as a dry powder to treat chronic infection and/or potentiate the effect of a co-administered antibiotic.

### Example 4: Intravenous Administration

In situations where it is not possible to administer artificial sweeteners to a patient orally, they can be administered intravenously. This preparation can be an additive to a standard rehydration fluid drip or can be a separate solution where the artificial sweetener is the sole active component solubilised in a saline solution.

Intravenous administration is schematically illustrated in FIG. 10. Intravenous solutions 40 and/or oral solutions 42 of the artificial sweetener, at concentrations that inhibit bacterial growth/virulence, can be administered to a patient, for example to treat/prevent bacteraemia and/or sepsis. In some examples, the artificial sweetener solutions are provided at concentrations that are insufficient to have antimicrobial effect, but sufficient to augment the effect of a co-administered antibiotic 44.

### Example 5: Growth Inhibition Effect

The effect on bacterial growth of a selection of artificial sweeteners was investigated. A standard nutrient medium was supplemented with 2.66% of each artificial sweetener. Control cultures were supplemented with an equal volume of the vehicle (dH₂0). Two specific opportunistic multidrug resistant clinically relevant pathogens were chosen for this assay: P. aeruginosa PA14 and A. baumannii AB5075. A. baumannii and P. aeruginosa occupy positions one and two in the WHO priority pathogen list respectively.

Cultures were incubated at 37° C. with shaking, and growth was monitored over time. Growth of P. aeruginosa PA14 was measured in the presence of 2.66% sweetener for 19 hours. As shown in FIGS. 11, 13, 15, 17, 19, 21, and 23, xylitol, sorbitol, sodium cyclamate, sucralose, maltitol, sodium saccharin and ace-K inhibit the growth of P. aeruginosa. The data depict the mean of three biological replicates±SD. FIGS. 12, 14, 16, 18, 20, 22, and 24, show growth at 19 hours. All of the sweeteners tested inhibit the bacterial growth with significant levels. The data present the mean of three biological replicates±SD. **p≤0.01, ***p≤0.001 versus the bacterial growth in control samples.

Growth of A. baumannii AB5075 was measured in the presence of 2.66% sweetener for 19 hours in LB medium. As shown in FIGS. 25, 27, 29, 31, 33, 35, 37, 39, and 41, xylitol, mannitol, erythritol, sodium cyclamate, sucralose, maltitol, lactitol monohydrate, sodium saccharin and Ace-K inhibit the growth of this pathogen. The data depict the mean of three biological replicates±SD. FIGS. 26, 28, 30, 32, 34, 36, 38, 40 and 42 show growth at 19 hours. All of the sweeteners tested inhibit the bacterial growth with significant levels. The data present the mean of 3 biological replicates±SD. **p≤0.01, ***p≤0.001 versus the bacterial growth in control samples.

FIGS. 11 to 42 thus demonstrate that xylitol, sodium cyclamate, sucralose, maltitol, sodium saccharin and ace-K all significantly inhibit the growth of P. aeruginosa and A. baumannii, with the most pronounced effects being seen with ace-K, sodium cyclamate, saccharin and sucralose. Furthermore, mannitol, meso-erythritol, and lactitol monohydrate had a significant impact on A. baumannii and sorbitol had a significant impact on P. aeruginosa.

To determine if this effect was dose-dependent, a minimum inhibitory concentration assay was performed for both pathogens exposing them to increasing concentrations of ace-K ranging from 0.09% to 7.08% (w/v).

Bacterial growth was tested in 10 different concentrations of ace-K (0.09-7.08%). The results are shown in FIG. 43 for P. aeruginosa and FIG. 44 for A. baumannii. A statistically significant level of inhibition can be observed at 0.89% and onwards in both bacterial species. The data present the mean of three biological replicates±SD. Data analysis by independent one way ANOVA—with Tukey's post-hoc multiple comparison test to compare pairs. *p≤0.05, **p≤0.01***p≤0.001 versus the bacterial growth in control samples.

A significant impact on growth was seen at 0.89% for both pathogens and the effect increased with increasing concentration, plateauing at around 5%. Visual analysis of the wells in this assay suggested no growth above this concentration.

To demonstrate that the antibacterial effect of artificial sweeteners is present in a broad range of sweeteners, the growth inhibition experiment included xylitol, mannitol, meso-erythritol, sodium cyclamate, sucralose, maltitol, sorbitol, lactitol monohydrate, sodium saccharin and ace-K. The data are three biological replicates (each with technical sextuplets), and the analysis of the endpoint OD₆₀₀ is by t-test.

FIGS. 64 to 67 show the results of growth inhibition assays for A. baumannii, P. aeruginosa, E. coli, and K. pneumonia using cyclamate (N=3 biological triplicate, each with technical triplicates; analysis by 2-way ANOVA). These all show significant growth inhibition at 2% cyclamate and above, with significant inhibition of E. coli growth occurring from 0.1% cyclamate.

A similar effect was seen with saccharin against E. coli, S. aureus, K. pneumonia, A. baumannii, and P. aeruginosa (see FIGS. 68-72; N=3 biological triplicate, each with technical triplicates). Effective growth inhibition can be observed at 2% saccharin and above for all tested organisms.

### Example 6: Biofilm Inhibition and Dispersal

Biofilm formation is linked to 80% of hospital-associated infections and is a major factor in the routine failure of antibiotic therapy. To determine if any of the artificial sweeteners under study could inhibit pathogen biofilm formation, a biofilm assay was established using a 3% (w/v) preparation of sucralose and 2.66% (w/v) preparation of ace-K. These concentrations were chosen as although they impacted growth for the artificial sweeteners in both pathogens, they did not completely inhibit growth, therefore it should be possible to resolve an impact on biofilm formation.

All samples were exposed to the artificial sweeteners in LB medium for 19 hours before crystal violet biofilm assay was performed. The results for P. aeruginosa PA14 are shown in FIG. 45 and the results for A. baumannii are shown in FIG. 46. Different graph scales were used for the different bacterial species due to the difference in their biofilm forming abilities. The data present the mean of three biological replicates±SD. *p≤0.05, *p≤0.001 versus the bacterial growth in control samples.

As can be seen from FIGS. 45 and 46, artificial sweeteners differently influence the formation of biofilm in both bacterial strains. Ace-K and sucralose can significantly inhibit biofilm formation by P. aeruginosa and A. baumannii.

To determine the full impact of ace-K on A. baumannii and P. aeruginosa biofilm formation a minimum biofilm inhibition concentration assay was performed. A range of ace-K concentrations was used. All samples were exposed to artificial sweeteners for 19 hours before crystal violet biofilm assay was performed.

The results are presented in FIGS. 47 (P. aeruginosa) and 48 (A. baumannii). The data present the mean of three biological replicates±SD. Data analysis by independent one-way ANOVA—with Tukey's post-hoc multiple comparison test to compare pairs. *p≤0.05, **p≤0.01***p≤0.001 versus the bacterial growth in control samples.

This assay revealed that at 0.09%, a significant impact on biofilm production could be seen in A. baumannii. This implies that ace-K has anti-virulence properties as well as antibacterial properties as the concentration is below that which impacts bacterial growth. For P. aeruginosa a concentration of 1.77% resulted in an almost complete abolition of biofilm formation.

Cyclamate and saccharin were also tested. FIGS. 73-75 (N=3 biological triplicate, each with technical triplicates) show that saccharin inhibits biofilm formation in K. pneumonia, P. aeruginosa, and A. baumannii at 2% and above, with some effect on P. aeruginosa being seen from about 0.5%. FIGS. 76-79 (N=3 biological triplicate, each with technical triplicates; analysis by 2-way ANOVA) show the results of cyclamate inhibition of biofilm formation in A. baumannii, P. aeruginosa, E. coli, and K. pneumonia. It can be seen that cyclamate inhibits biofilm formation in all species, at a concentration of 3% and above in E. coli and K. pneumonia, from 2% in P. aeruginosa and from 4% in A. baumannii.

### Example 7: Impact of Ace-K, Saccharin and Cyclamate on Gene Expression

Given that the most pronounced effects on growth and biofilm formation were observed for A. baumannii and ace-K, RNA-seq analysis was performed to determine the influence ace-K had over gene expression in A. baumannii. Cells were grown to early exponential phase (OD 0.6-0.7) in 20 ml LB supplemented with either 1.34% ace-K or the matching volume of vehicle control. Cells were spun down and washed in RNAlater to preserve mRNA. RNA was isolated using a Qiagen RNAeasy Kit with column DNAase digestion. RNA integrity was determined using a Bioanalyzer. Samples were further processed for RNA sequencing on an Illumina MiSeq with 12 million reads per sample. Quality control and adapter trimming was performed with bcl2fastq. Read mapping was performed with HISAT. Differential expression analysis was performed using edgeR's exact test for differences between two groups of negative-binomial counts with an estimated dispersion value of 0.1. 464 genes were identified as being significantly differentially expressed greater than |logFC|>1 and p<.05 (Table 1).

TABLE 1

| Locustag | Gene | logFC | PValue |
|---|---|---|---|
| ABUW_0020 | | −1.466497832 | 0.000187017 |
| ABUW_0030 | | 1.131184384 | 0.002567219 |
| ABUW_0031 | ppc | −1.662752483 | 5.08E−05 |
| ABUW_0066 | hppD | 2.752392366 | 2.46E−06 |
| ABUW_0068 | | 2.934595319 | 1.34E−05 |
| ABUW_0069 | maiA | 3.066678875 | 1.43E−06 |
| ABUW_0070 | fahA | 2.656580577 | 1.24E−05 |
| ABUW_0071 | aroP1 | 2.705121681 | 4.33E−05 |
| ABUW_0072 | | 1.649882584 | 2.51E−05 |
| ABUW_0083 | | −1.169711141 | 0.000348967 |
| ABUW_0088 | | −1.30717283 | 8.34E−05 |
| ABUW_0104 | | −1.392962697 | 0.00140037 |
| ABUW_0117 | | −1.016081429 | 0.005726823 |
| ABUW_0121 | | −1.608118722 | 0.000883377 |
| ABUW_0133 | | −1.590578804 | 0.000200506 |
| ABUW_0143 | | 2.019352617 | 7.77E−06 |
| ABUW_0144 | | −1.157897762 | 0.02207712 |
| ABUW_0154 | | 1.073665908 | 0.000275842 |
| ABUW_0160 | | −2.548549696 | 1.48E−05 |
| ABUW_0203 | gabT | −1.121557601 | 0.00305788 |
| ABUW_0225 | | −1.53286278 | 9.57E−05 |
| ABUW_0248 | | −2.784516767 | 4.44E−05 |
| ABUW_0259 | | 1.866484379 | 0.002857042 |
| ABUW_0263 | | −3.533749325 | 1.51E−05 |
| ABUW_0280 | sbp | −2.180402608 | 3.20E−05 |
| ABUW_0290 | | −2.750354273 | 0.000244176 |
| ABUW_0291 | comN | −3.437791867 | 5.85E−05 |
| ABUW_0292 | comO | −3.180420132 | 0.000111766 |
| ABUW_0293 | comL | −3.553965663 | 6.75E−05 |
| ABUW_0294 | comQ | −3.182923601 | 3.19E−05 |
| ABUW_0304 | | −5.413509044 | 3.66E−07 |
| ABUW_0306 | bfr1 | −1.130984988 | 0.011409578 |
| ABUW_0307 | | 2.72139885 | 0.006597702 |
| ABUW_0313 | fimT | −2.408510092 | 0.000368468 |
| ABUW_0314 | pilV | −3.197287217 | 9.00E−05 |
| ABUW_0315 | pilW | −2.646553666 | 0.00018702 |
| ABUW_0316 | pilX | −2.351181112 | 0.000105213 |
| ABUW_0317 | pilY | −2.569853292 | 2.03E−05 |
| ABUW_0318 | comE | −2.118684292 | 0.000111502 |
| ABUW_0319 | comF | −1.822946469 | 3.28E−05 |
| ABUW_0356 | | 2.685891775 | 1.51E−06 |
| ABUW_0375 | | −1.01121916 | 0.000429532 |
| ABUW_0377 | hemF | −1.134263602 | 0.014706827 |
| ABUW_0386 | mlaC | 1.15915729 | 0.000422654 |
| ABUW_0387 | mlaB | 1.126772992 | 0.000792293 |
| ABUW_0437 | | −1.181318166 | 0.001922308 |
| ABUW_0461 | | −1.366538508 | 0.000126973 |
| ABUW_0469 | | −1.260191826 | 0.001185083 |
| ABUW_0498 | | −1.038870197 | 0.002687295 |
| ABUW_0514 | | −2.071185747 | 0.000142172 |
| ABUW_0527 | | −1.188703686 | 4.62E−05 |
| ABUW_0528 | radC | −1.053311891 | 0.000121621 |
| ABUW_0534 | | −2.644159862 | 4.46E−06 |
| ABUW_0535 | | −2.125038625 | 1.00E−05 |
| ABUW_0547 | | 1.198585588 | 3.93E−05 |
| ABUW_0548 | | 1.070564147 | 0.001867164 |
| ABUW_0550 | | −1.029404612 | 0.00020632 |
| ABUW_0553 | | −1.367693461 | 0.000449378 |
| ABUW_0554 | | −1.250883348 | 0.000129916 |
| ABUW_0571 | | −1.838461955 | 0.00210141 |
| ABUW_0585 | | −1.311483704 | 0.000560258 |
| ABUW_0600 | | 1.204184781 | 0.000165549 |
| ABUW_0603 | | 6.286028154 | 5.83E−07 |
| ABUW_0607 | | 4.309477176 | 5.47E−07 |
| ABUW_0641 | | −1.523170143 | 0.000156602 |
| ABUW_0642 | gcd | −1.184166008 | 0.000775223 |
| ABUW_0648 | | −1.261801918 | 0.00057305 |
| ABUW_0677 | | −2.24450084 | 5.83E−05 |
| ABUW_0678 | pilG | −1.868612819 | 0.000255176 |
| ABUW_0679 | pilH | −1.947111824 | 9.77E−05 |
| ABUW_0680 | pilI | −2.789060235 | 8.03E−05 |
| ABUW_0681 | pilJ | −3.199017969 | 4.03E−06 |
| ABUW_0682 | pilL | −3.848722689 | 1.28E−06 |
| ABUW_0683 | | −3.425360452 | 1.54E−06 |
| ABUW_0684 | | −2.620054821 | 1.51E−06 |
| ABUW_0685 | | −1.423469858 | 1.74E−05 |
| ABUW_0697 | | 1.439664341 | 0.00409279 |
| ABUW_0698 | | 2.032140891 | 2.22E−06 |
| ABUW_0705 | | 1.408724375 | 0.000262208 |
| ABUW_0728 | | −1.19990768 | 0.000278044 |
| ABUW_0729 | uppP | 1.257424484 | 0.001323018 |
| ABUW_0742 | | 1.317399781 | 0.000384745 |
| ABUW_0743 | | 1.011711229 | 0.044274182 |
| ABUW_0750 | | 1.194575167 | 0.025376855 |
| ABUW_0767 | | −1.38592468 | 0.025841897 |
| ABUW_0771 | | −1.749765818 | 0.000294582 |
| ABUW_0795 | | −1.392995264 | 0.010427485 |
| ABUW_0796 | | −1.412632373 | 0.005070057 |
| ABUW_0887 | | −1.039491691 | 0.000184572 |
| ABUW_0888 | | −1.142953586 | 0.00770559 |
| ABUW_0894 | carA | 1.00556565 | 0.011792485 |
| ABUW_0916 | | −2.661406111 | 1.88E−05 |
| ABUW_0917 | groS | −1.215020226 | 0.001572196 |
| ABUW_0918 | groL | −1.331934074 | 0.000483765 |
| ABUW_0922 | | 1.842828743 | 3.61E−06 |
| ABUW_0926 | | 1.123279454 | 0.009745085 |
| ABUW_0928 | | 1.510330403 | 0.000333789 |
| ABUW_0935 | | −1.196099708 | 0.003186304 |
| ABUW_0965 | | −1.032171868 | 0.005720582 |
| ABUW_0967 | | −1.037938043 | 0.001303405 |
| ABUW_0988 | | 1.147960065 | 0.000150924 |
| ABUW_0995 | tolR | 1.312687305 | 0.000234406 |
| ABUW_0996 | tolQ | 1.291460198 | 0.00010881 |
| ABUW_0998 | | 1.026078652 | 0.000589691 |
| ABUW_1011 | | 1.098024483 | 0.002166601 |
| ABUW_1020 | | −2.094522952 | 0.000110247 |
| ABUW_1021 | cysP | −1.632496106 | 0.00616196 |
| ABUW_1022 | pabC | −1.024023044 | 0.001655159 |
| ABUW_1041 | | −1.560805485 | 1.43E−05 |
| ABUW_1066 | | −1.201410829 | 0.044664516 |
| ABUW_1067 | phrB | −1.227932156 | 1.97E−05 |
| ABUW_1087 | cas | −1.541574497 | 0.000301128 |
| ABUW_1088 | | −1.489893664 | 3.71E−05 |
| ABUW_1089 | csy1 | −1.19474486 | 0.000558167 |
| ABUW_1090 | | −1.329412921 | 4.87E−05 |
| ABUW_1091 | | −1.484662638 | 6.72E−05 |
| ABUW_1092 | csy4 | −1.352374437 | 0.000124467 |
| ABUW_1111 | | 1.601098487 | 0.001170337 |
| ABUW_1113 | | 2.638797209 | 6.70E−05 |
| ABUW_1114 | aroP3 | 3.132014953 | 5.53E−06 |
| ABUW_1120 | | −1.076859068 | 0.004746354 |
| ABUW_1129 | | −1.22179669 | 0.00954948 |
| ABUW_1132 | | −1.040208412 | 0.000193867 |
| ABUW_1138 | | −1.758445041 | 7.39E−06 |
| ABUW_1139 | | −1.224248523 | 0.00010996 |
| ABUW_1142 | cstA | −1.031329213 | 0.002189473 |
| ABUW_1143 | | −1.032618412 | 0.000540916 |
| ABUW_1156 | | −1.111968322 | 0.006233951 |
| ABUW_1163 | | −1.022485992 | 0.048500326 |
| ABUW_1168 | bauF | 1.636606598 | 0.000176441 |
| ABUW_1180 | basE | 1.068820945 | 0.000699548 |
| ABUW_1192 | | −2.219288343 | 1.84E−05 |
| ABUW_1206 | | 1.179604026 | 0.000634884 |
| ABUW_1233 | | 2.416925204 | 1.65E−05 |
| ABUW_1242 | rlpA | 1.188339178 | 0.000706682 |
| ABUW_1287 | | −1.380194228 | 0.037597085 |
| ABUW_1288 | | −1.296268367 | 0.000285374 |
| ABUW_1295 | | −1.156341493 | 0.000512996 |
| ABUW_1300 | | 1.512708693 | 0.007488108 |
| ABUW_1315 | | −1.298965921 | 0.017195373 |
| ABUW_1337 | | −1.180354033 | 0.038044208 |
| ABUW_1351 | | −2.214334203 | 4.07E−06 |
| ABUW_1355 | | 1.959490855 | 7.41E−05 |
| ABUW_1364 | | 1.014621919 | 0.000521667 |
| ABUW_1394 | | −1.476628756 | 0.011995681 |
| ABUW_1453 | | 1.275835965 | 0.000504302 |
| ABUW_1463 | | −1.52636202 | 0.001243476 |
| ABUW_1487 | | −1.30395562 | 0.000687199 |
| ABUW_1488 | | −1.780623504 | 0.001243239 |
| ABUW_1490 | | −1.247671737 | 0.002216674 |
| ABUW_1492 | | −1.117326306 | 0.004710343 |
| ABUW_1499 | | −1.019717195 | 0.010168875 |
| ABUW_1515 | | −1.08361202 | 0.000543387 |
| ABUW_1518 | | −1.105849987 | 0.000565342 |
| ABUW_1533 | fis | 1.265091712 | 0.001313107 |
| ABUW_1537 | gidA | 1.09995293 | 0.01193766 |
| ABUW_1562 | | 1.284534477 | 2.56E−05 |
| ABUW_1563 | | 4.678512038 | 2.98E−08 |
| ABUW_1564 | | 3.318153338 | 1.98E−06 |
| ABUW_1594 | | −1.682291687 | 0.001047994 |
| ABUW_1595 | | −1.107236052 | 0.000121872 |
| ABUW_1597 | | −1.652528668 | 0.00114782 |
| ABUW_1598 | | 1.065320587 | 0.000941799 |
| ABUW_1624 | | −1.075482259 | 0.03402707 |
| ABUW_1629 | | 2.404390167 | 0.002377808 |
| ABUW_1631 | | −2.204814642 | 0.026489245 |
| ABUW_1632 | | −2.482131151 | 0.015432172 |
| ABUW_1633 | | −2.643520292 | 0.012323004 |
| ABUW_1634 | | −2.663675129 | 0.009732628 |
| ABUW_1635 | | −2.784088166 | 0.003186104 |
| ABUW_1636 | | −1.956444312 | 0.008324594 |
| ABUW_1637 | | −1.767351233 | 0.010744488 |
| ABUW_1646 | | 1.68958928 | 0.00065342 |
| ABUW_1651 | | −2.206721787 | 0.021735769 |
| ABUW_1652 | | 1.21351914 | 0.01413778 |
| ABUW_1655 | | 1.78294092 | 1.29E−05 |
| ABUW_1661 | | −1.103969785 | 0.003068735 |
| ABUW_1664 | | −1.034009586 | 0.000253631 |
| ABUW_1668 | | −1.129413728 | 0.000587172 |
| ABUW_1685 | | 1.048207846 | 0.000383534 |
| ABUW_1691 | | 1.225840724 | 0.000501161 |
| ABUW_1693 | | −1.281266809 | 0.001299335 |
| ABUW_1722 | fumC | 1.041232013 | 0.012262649 |
| ABUW_1724 | | −1.265618046 | 0.0002817 |
| ABUW_1734 | | 1.006732263 | 0.009545098 |
| ABUW_1769 | | −2.134001852 | 0.000536672 |
| ABUW_1776 | | 1.141079783 | 0.002851236 |
| ABUW_1792 | | −1.212659801 | 0.00420855 |
| ABUW_1793 | ybgT | −1.153865437 | 0.004098842 |
| ABUW_1794 | cydB | −1.323395251 | 0.001904854 |
| ABUW_1795 | cydA | −1.276009794 | 0.001105237 |
| ABUW_1799 | | 1.38006428 | 0.002916889 |
| ABUW_1800 | | 1.62348714 | 0.004538129 |
| ABUW_1854 | benP1 | 1.661172111 | 0.001349593 |
| ABUW_1855 | | 3.660939107 | 7.37E−06 |
| ABUW_1857 | | 5.797107545 | 1.55E−07 |
| ABUW_1858 | | 5.579215648 | 6.39E−09 |
| ABUW_1859 | | 4.685904442 | 1.65E−08 |
| ABUW_1860 | | 5.179132031 | 8.31E−08 |
| ABUW_1861 | | 5.0270445 | 4.91E−09 |
| ABUW_1862 | | 4.860464357 | 2.59E−08 |
| ABUW_1863 | | 4.757178158 | 7.31E−08 |
| ABUW_1864 | | 4.739255823 | 3.42E−08 |
| ABUW_1865 | | 4.59045567 | 2.49E−08 |
| ABUW_1866 | feaR | 1.355182017 | 0.001231473 |
| ABUW_1870 | | 3.072270076 | 9.15E−07 |
| ABUW_1871 | pcaD2 | 2.016407073 | 0.00018704 |
| ABUW_1872 | pcaF2 | 2.97031336 | 3.27E−05 |
| ABUW_1873 | pcaJ2 | 2.493530869 | 1.13E−05 |
| ABUW_1874 | pcaI2 | 2.897925321 | 5.16E−06 |
| ABUW_1875 | catA | 2.848890679 | 1.91E−05 |
| ABUW_1876 | catC | 3.431764896 | 2.51E−07 |
| ABUW_1877 | catB | 3.365603183 | 3.18E−06 |
| ABUW_1902 | sndH2 | 1.022823752 | 0.038260935 |
| ABUW_1904 | | 1.839890133 | 0.000168846 |
| ABUW_1905 | | 1.934037113 | 9.99E−05 |
| ABUW_1923 | | 1.621995508 | 7.52E−06 |
| ABUW_1930 | | 1.696966402 | 1.74E−05 |
| ABUW_1931 | | 1.802440069 | 0.000102266 |
| ABUW_1943 | | −1.0296694 | 0.000680154 |
| ABUW_1945 | | 1.628124427 | 0.000603731 |
| ABUW_1947 | | 1.116568789 | 0.001762959 |
| ABUW_1951 | | −1.572806181 | 7.52E−05 |
| ABUW_1952 | | −1.645056157 | 0.000221174 |
| ABUW_1958 | | −1.065626414 | 0.012167367 |
| ABUW_2051 | | −2.110920362 | 6.41E−06 |
| ABUW_2052 | | −1.082817552 | 0.000282893 |
| ABUW_2058 | | −1.284972938 | 0.044844424 |
| ABUW_2074 | | 1.268808884 | 0.010986351 |
| ABUW_2092 | bdhA | 1.070268873 | 0.003984814 |
| ABUW_2093 | | 1.096680662 | 0.001265447 |
| ABUW_2096 | atoD | 2.069583731 | 2.61E−05 |
| ABUW_2097 | atoA | 1.758931297 | 0.000358444 |
| ABUW_2098 | atoE | 1.933346074 | 3.01E−05 |
| ABUW_2099 | | 1.395295785 | 0.000117896 |
| ABUW_2103 | | 1.460433919 | 0.019375197 |
| ABUW_2104 | aspA | 3.542361361 | 1.13E−07 |
| ABUW_2126 | | 1.549159738 | 0.000463005 |
| ABUW_2127 | budC | 2.321771164 | 6.80E−05 |
| ABUW_2128 | lpdA2 | 3.423835882 | 1.60E−05 |
| ABUW_2129 | acoC | 3.334478552 | 7.78E−06 |
| ABUW_2130 | acoB | 3.347317387 | 1.81E−05 |
| ABUW_2131 | acoA | 3.304022616 | 1.13E−05 |
| ABUW_2132 | lipA2 | 1.448735917 | 0.001066703 |
| ABUW_2133 | | 1.721961366 | 0.000109502 |
| ABUW_2137 | prmB | 1.29523745 | 0.006554191 |
| ABUW_2145 | | −1.915367741 | 0.000959293 |
| ABUW_2149 | | −1.056180418 | 0.029351041 |
| ABUW_2165 | | 2.768535583 | 3.37E−07 |
| ABUW_2166 | | 3.295468901 | 2.27E−05 |
| ABUW_2167 | | 1.73049677 | 7.32E−05 |
| ABUW_2168 | | 1.360754013 | 0.001498691 |
| ABUW_2178 | | 1.895019127 | 0.000305177 |
| ABUW_2180 | | 1.223333605 | 3.78E−05 |
| ABUW_2181 | | 1.242743004 | 0.000229057 |
| ABUW_2182 | | 1.82160079 | 0.000131338 |
| ABUW_2183 | | 1.249511951 | 0.002886703 |
| ABUW_2184 | | 1.643835638 | 0.002699915 |
| ABUW_2185 | | 2.172475135 | 0.001419644 |
| ABUW_2186 | | 2.225129098 | 0.000845653 |
| ABUW_2187 | | 2.634293548 | 0.000149139 |
| ABUW_2188 | | 2.761612691 | 4.98E−05 |
| ABUW_2189 | | 3.219106199 | 2.88E−05 |
| ABUW_2224 | | −1.048075899 | 0.001759372 |
| ABUW_2229 | | −1.123658535 | 0.002412254 |
| ABUW_2230 | | −1.154467846 | 0.000822803 |
| ABUW_2231 | | −1.308586895 | 0.002814709 |
| ABUW_2255 | pilZ | −1.438583788 | 0.001192124 |
| ABUW_2285 | putP | −1.664355186 | 0.001833819 |
| ABUW_2287 | putA | −5.078189662 | 1.75E−08 |
| ABUW_2289 | trmA | 1.067071984 | 0.009562089 |
| ABUW_2300 | bla-OXA-69 | 1.259264841 | 0.000228993 |
| ABUW_2310 | | −2.564330103 | 1.91E−05 |
| ABUW_2311 | | −1.711192739 | 4.57E−05 |
| ABUW_2312 | | −1.296447299 | 0.000358113 |
| ABUW_2313 | | −1.411186882 | 1.85E−05 |
| ABUW_2316 | | 1.49063155 | 0.000393493 |
| ABUW_2325 | | −1.848003071 | 3.33E−06 |
| ABUW_2342 | ychF | 1.327962058 | 0.006436234 |
| ABUW_2349 | | −1.269300964 | 0.000888955 |
| ABUW_2358 | | 2.981655599 | 9.58E−07 |
| ABUW_2359 | aspQ | 1.554817446 | 0.000758951 |
| ABUW_2380 | tauC | −1.330148404 | 0.000770956 |
| ABUW_2381 | tauB | −2.336963784 | 6.94E−05 |
| ABUW_2382 | tauA | −1.958047571 | 0.001449207 |
| ABUW_2385 | | −1.228676776 | 4.56E−05 |
| ABUW_2387 | | 1.010340515 | 0.001846074 |
| ABUW_2402 | mdcA | 1.150768787 | 0.000829356 |
| ABUW_2420 | | −1.476688883 | 0.000577458 |
| ABUW_2421 | | −1.440104081 | 7.57E−05 |
| ABUW_2422 | | −1.576031907 | 0.000643888 |
| ABUW_2423 | | −2.356991151 | 0.000485959 |
| ABUW_2424 | | −1.866897947 | 0.00012927 |
| ABUW_2430 | | −1.652169438 | 0.000207431 |
| ABUW_2441 | | −1.505022601 | 0.013586012 |
| ABUW_2442 | | −1.918279238 | 0.023499836 |
| ABUW_2457 | | 1.04514394 | 0.002159879 |
| ABUW_2521 | | −1.173894984 | 0.000968673 |
| ABUW_2522 | | −1.209255071 | 0.00196152 |
| ABUW_2525 | paaX | 1.08722417 | 0.014818767 |
| ABUW_2526 | paaF | 1.496489524 | 0.015607933 |
| ABUW_2527 | paaE | 1.587992108 | 0.027953223 |
| ABUW_2528 | paaC | 1.534830488 | 0.044002061 |
| ABUW_2529 | paaB | 2.064594312 | 0.011966194 |
| ABUW_2530 | caiD | 2.746093291 | 0.004774612 |
| ABUW_2531 | paak | 2.866510862 | 0.002699749 |
| ABUW_2532 | paaJ | 2.501751658 | 0.003241118 |
| ABUW_2533 | paaI2 | 2.439004051 | 0.003451069 |
| ABUW_2534 | paaH | 2.365885667 | 0.001435415 |
| ABUW_2535 | paaG | 2.435418923 | 8.71E−05 |
| ABUW_2536 | paaN | 2.16868625 | 0.000854826 |
| ABUW_2553 | | −1.288891336 | 0.018845979 |
| ABUW_2598 | | −1.39677931 | 0.000432429 |
| ABUW_2599 | | −1.675434378 | 0.001630028 |
| ABUW_2604 | | 1.163399871 | 0.021853168 |
| ABUW_2605 | | 1.017596325 | 0.027567766 |
| ABUW_2621 | | −2.912474378 | 0.00201047 |
| ABUW_2627 | | −1.199276171 | 0.000110605 |
| ABUW_2628 | pcaK2 | 1.431957026 | 7.85E−05 |
| ABUW_2637 | | 1.06614956 | 0.000477291 |
| ABUW_2655 | | −2.981342343 | 4.63E−07 |
| ABUW_2656 | | −1.194046468 | 5.24E−05 |
| ABUW_2658 | | −2.317327461 | 0.000150706 |
| ABUW_2665 | | −1.586713237 | 0.000241737 |
| ABUW_2666 | | −1.922854252 | 0.000391577 |
| ABUW_2673 | | −1.57291673 | 0.041728508 |
| ABUW_2681 | | 1.284954904 | 0.010300249 |
| ABUW_2689 | | −1.45083751 | 0.000227783 |
| ABUW_2696 | | 1.257686214 | 0.020734222 |
| ABUW_2710 | benA | 2.535332459 | 2.75E−05 |
| ABUW_2711 | benB | 2.254429316 | 0.00150655 |
| ABUW_2712 | benC | 1.335210531 | 0.000914149 |
| ABUW_2713 | benD | 1.096812304 | 0.001908308 |
| ABUW_2727 | trmB | 1.328999692 | 0.007700896 |
| ABUW_2733 | | −1.13014029 | 0.000736856 |
| ABUW_2737 | clpB | −1.369007265 | 0.000956498 |
| ABUW_2756 | | −1.605815093 | 0.000676067 |
| ABUW_2770 | | −1.649194767 | 0.000439367 |
| ABUW_2790 | | 1.045264205 | 0.000517961 |
| ABUW_2795 | | −1.139244287 | 0.01187806 |
| ABUW_2796 | | −1.273995412 | 0.003146222 |
| ABUW_2797 | | −1.113958432 | 0.003724269 |
| ABUW_2806 | | 1.021154946 | 0.000771534 |
| ABUW_2811 | | −1.006950077 | 0.022496507 |
| ABUW_2813 | | −1.073061401 | 0.031549755 |
| ABUW_2828 | | 2.394610154 | 1.22E−06 |
| ABUW_2830 | | −1.16523381 | 0.000703221 |
| ABUW_2831 | | −1.121858372 | 0.003080071 |
| ABUW_2834 | | −1.09855533 | 0.002121165 |
| ABUW_2842 | | −2.692061917 | 1.44E−06 |
| ABUW_2870 | | 1.125973202 | 0.006847745 |
| ABUW_2888 | aceA | −1.087663223 | 0.001068549 |
| ABUW_2900 | | −1.018390017 | 0.000322589 |
| ABUW_2916 | pfeA | 1.075575281 | 8.05E−05 |
| ABUW_2922 | | 1.576785275 | 0.00131552 |
| ABUW_2936 | | −1.051265921 | 9.39E−05 |
| ABUW_2939 | | 1.497822425 | 9.59E−06 |
| ABUW_2953 | | 1.223620767 | 0.006141378 |
| ABUW_2962 | | 1.027871496 | 0.008096778 |
| ABUW_2964 | | 1.434899231 | 4.74E−05 |
| ABUW_2965 | | 1.91181124 | 0.001809597 |
| ABUW_2967 | | 1.096909784 | 0.001120818 |
| ABUW_2968 | betT | 1.256092388 | 0.000139691 |
| ABUW_2969 | | 1.329102285 | 1.98E−05 |
| ABUW_2973 | mqo | 1.325449026 | 0.00477832 |
| ABUW_2975 | | −1.135415929 | 0.006837081 |
| ABUW_2986 | | 1.439763831 | 0.000323493 |
| ABUW_2987 | | 1.561653705 | 0.000406919 |
| ABUW_2993 | | −1.190027669 | 0.000380529 |
| ABUW_3011 | | −1.246980061 | 0.014399214 |
| ABUW_3016 | | −1.00416816 | 0.000161276 |
| ABUW_3019 | emrB | 1.576381967 | 1.17E−05 |
| ABUW_3020 | emrA | 1.922892937 | 1.78E−06 |
| ABUW_3031 | pilT | −2.310481188 | 2.49E−06 |
| ABUW_3032 | pilU | −2.547722755 | 6.37E−07 |
| ABUW_3037 | | −2.22662594 | 0.000175737 |
| ABUW_3101 | | −1.233800712 | 0.003155283 |
| ABUW_3102 | | −1.291039534 | 0.003393732 |
| ABUW_3124 | | 1.182120508 | 0.026330854 |
| ABUW_3125 | bfr2 | −1.30495152 | 0.005367664 |
| ABUW_3162 | | −1.004596429 | 0.000734439 |
| ABUW_3182 | | −1.318246702 | 0.000571711 |
| ABUW_3186 | | 2.33599907 | 9.14E−05 |
| ABUW_3191 | | −1.826120678 | 0.000626047 |
| ABUW_3192 | | −2.147306764 | 0.002232295 |
| ABUW_3193 | | −1.062191158 | 0.001925156 |
| ABUW_3204 | | −1.774507562 | 0.000531221 |
| ABUW_3286 | | 1.675035131 | 3.81E−05 |
| ABUW_3332 | | −1.192400584 | 0.016835846 |
| ABUW_3337 | | −1.486084593 | 7.78E−06 |
| ABUW_3342 | dusA | 1.211677065 | 0.000464639 |
| ABUW_3343 | | 2.522352648 | 4.82E−06 |
| ABUW_3344 | | 1.02241681 | 0.000230469 |
| ABUW_3351 | | −2.050059891 | 1.90E−05 |
| ABUW_3358 | | −2.169352924 | 1.30E−05 |
| ABUW_3362 | | 1.647924064 | 1.50E−05 |
| ABUW_3363 | macB | 1.211669142 | 4.91E−05 |
| ABUW_3364 | | 1.279225684 | 5.18E−05 |
| ABUW_3396 | pta | −1.051048081 | 0.003659447 |
| ABUW_3403 | | 2.085710276 | 0.001970168 |
| ABUW_3424 | | 1.247617239 | 0.000280309 |
| ABUW_3425 | | 1.601687763 | 7.66E−05 |
| ABUW_3426 | | 1.86329375 | 1.30E−05 |
| ABUW_3439 | | −1.262171408 | 0.000141913 |
| ABUW_3453 | | −2.680084197 | 3.24E−05 |
| ABUW_3459 | | −1.930952177 | 4.23E−06 |
| ABUW_3487 | | −1.069963228 | 0.000900451 |
| ABUW_3488 | | −1.059660199 | 0.000271872 |
| ABUW_3499 | | −1.076848539 | 0.000366387 |
| ABUW_3514 | | −1.422807477 | 4.57E−05 |
| ABUW_3530 | | −1.188567309 | 0.000644493 |
| ABUW_3549 | pilB | −2.443848945 | 0.000121919 |
| ABUW_3550 | pilC | −2.608063399 | 0.000176643 |
| ABUW_3551 | pilD | −1.40303889 | 0.001110607 |
| ABUW_3560 | | 1.492538144 | 5.54E−05 |
| ABUW_3561 | | 2.448738584 | 0.000149119 |
| ABUW_3562 | | 2.380654266 | 1.31E−06 |
| ABUW_3571 | | −2.014921895 | 0.003283463 |
| ABUW_3579 | | −1.178798443 | 0.005939728 |
| ABUW_3588 | | 1.255206501 | 0.000400077 |
| ABUW_3622 | | 1.305929983 | 0.000398844 |
| ABUW_3633 | feoA | 1.080397955 | 0.007001617 |
| ABUW_3641 | pilR | −2.233979273 | 4.32E−06 |
| ABUW_3706 | | −1.090558097 | 0.001381673 |
| ABUW_3719 | | 1.106906107 | 0.000327954 |
| ABUW_3722 | | −1.725802982 | 0.000238975 |
| ABUW_3723 | dprA | −1.724800459 | 0.000210078 |
| ABUW_3730 | | 1.198680597 | 0.000232347 |
| ABUW_3777 | | 2.040025143 | 5.26E−05 |
| ABUW_3781 | | 3.068956725 | 8.83E−05 |
| ABUW_3782 | mmsB | 2.55114889 | 0.000586866 |
| ABUW_3783 | mmsA1 | 2.645326526 | 0.000153743 |
| ABUW_3787 | | −1.576076275 | 0.000667821 |
| ABUW_3788 | dadX | −1.156138247 | 0.001186136 |
| ABUW_3789 | dadA2 | −1.237507612 | 0.001765981 |
| ABUW_3797 | | −1.386412119 | 0.007701785 |
| ABUW_3798 | | −2.452729969 | 3.12E−06 |
| ABUW_3803 | | −1.015795252 | 0.007815163 |
| ABUW_3804 | | −1.399747108 | 0.005873496 |
| ABUW_3805 | | −1.259125525 | 0.000257829 |
| ABUW_3811 | dld | −3.015086242 | 6.09E−08 |
| ABUW_3812 | lldD | −3.450472456 | 1.33E−07 |
| ABUW_3813 | lldR | −3.254882636 | 9.47E−08 |
| ABUW_3814 | lldP | −5.111652801 | 1.46E−08 |
| ABUW_3838 | nadC | 1.166238698 | 0.000858396 |
| ABUW_3839 | | 2.232228138 | 3.63E−05 |
| ABUW_3851 | | 4.225812632 | 2.22E−06 |
| ABUW_3874 | | 2.210294507 | 0.000304541 |
| ABUW_3875 | | 1.413495025 | 0.001884202 |
| ABUW_3878 | grpE | −1.077426106 | 0.000327062 |
| ABUW_3879 | dnak | −1.146049607 | 0.000789385 |
| ABUW_3880 | | −1.19152474 | 0.000702505 |
| ABUW_3884 | | −1.235183269 | 0.013983442 |
| ABUW_3887 | | −1.058275224 | 0.001531793 |
| ABUW_3892 | | −1.19783562 | 0.000176112 |
| ABUW_3899 | dsbC2 | 1.05305784 | 0.001053195 |
| ABUW_4021 | | 2.021860601 | 0.00019345 |
| ABUW_4031 | | −1.412510812 | 0.000223039 |
| ABUW_4032 | | −1.546866393 | 0.003635406 |
| ABUW_4069 | | 1.247746584 | 0.004170976 |
| ABUW_4071 | | 1.030385032 | 0.000754874 |
| ABUW_4072 | | 1.181300821 | 0.000223564 |
| ABUW_4087 | | 1.022806857 | 0.006205437 |
| ABUW_4116 | | −1.066065598 | 0.000475097 |

Analysis of this panel of genes revealed that almost all genes involved in pilus production and natural transformation were significantly down-regulated (Table 2).

### Table 2

Table 2 shows differentially expressed genes associated with pilus assembly and function and natural competency. All genes were down-regulated compared to vehicle control.

| Locus Tag | Name | Function | Log2FC |
|---|---|---|---|
| ABUW_0290 | | type IV pilus assembly protein | −2.7504 |
| | | PilM | |
| ABUW_0291 | comN | type 4 fimbrial biogenesis protein | −3.4378 |
| | | PilN | |
| ABUW_0292 | comO | pilus assembly protein, PilO | −3.1804 |
| ABUW_0293 | comL | pilus assembly protein, PilQ. | −3.554 |
| ABUW_0294 | comQ | fimbrial assembly protein PilQ | −3.1829 |
| ABUW_0304 | | type IV pilin structural subunit | −5.4135 |
| ABUW_0306 | bfrI | bacterioferritin | −1.131 |
| ABUW_0313 | fimT | pilin protein FimT | −2.4085 |
| ABUW_0314 | pilV | type IV pilus modification protein | −3.1973 |
| | | PilV | |
| ABUW_0315 | pilW | pilus assembly protein PilW | −2.6466 |
| ABUW_0316 | pilX | pilus assembly protein PilX | −2.3512 |
| ABUW_0317 | pilY | pilus assembly protein tip- | −2.5699 |
| | | associated adhesin PilY | |
| ABUW_0318 | comE | pilin like competence factor | −2.1187 |
| ABUW_0319 | comF | pilin like competence factor | −1.823 |
| ABUW_0648 | | type 4 fimbrial biogenesis protein | −1.2618 |
| | | FimT | |
| ABUW_0677 | | hypothetical protein | −2.2445 |
| ABUW_0678 | pilG | type IV pilus response regulator | −1.8686 |
| | | receiver protein PilG | |
| ABUW_0679 | pilH | type IV pilus response regulator | −1.9471 |
| | | protein PilH | |
| ABUW_0680 | pilI | type IV pilus signal transduction | −2.7891 |
| | | protein PilI | |
| ABUW_0681 | pilJ | type IV pilus methyl-accepting | −3.199 |
| | | chemotaxis sensory tr | |
| ABUW_0682 | pilL | type IV pilus hybrid sensor | −3.8487 |
| | | kinase/response regulator | |
| ABUW_0683 | | hypothetical protein | −3.4254 |
| ABUW_0684 | | coproporphyrinogen III oxidase | −2.6201 |
| ABUW_0685 | | alpha/beta hydrolase fold protein | −1.4235 |
| ABUW_2255 | pilZ | type 4 fimbrial biogenesis protein | −1.4386 |
| ABUW_2310 | | fimbrial protein | −2.5643 |
| ABUW_2311 | | pili assembly chaperone | −1.7112 |
| ABUW_2312 | | fimbrial biogenesis outer | −1.2965 |
| | | membrane usher protein | |
| ABUW_2313 | | fimbrial protein | −1.4112 |
| ABUW_3031 | pilT | twitching mobility protein | −2.3105 |
| ABUW_3032 | pilU | twitching motility protein | −2.5477 |
| ABUW_3549 | pilB | type IV-A pilus assembly ATPase | −2.4439 |
| | | PilB | |
| ABUW_3550 | pilC | pilin biogenesis protein | −2.6081 |
| ABUW_3551 | pilD | type IV pilus prepilin peptidase | −1.403 |
| | | PilD | |
| ABUW_3641 | pilR | type 4 fimbriae expression | −2.234 |
| | | regulatory protein PilR | |

This was a particularly interesting finding as in both Yu et al. studies, ace-K increased the expression of these genes albeit at lower concentrations (Yu et al. (2021a); Yu et al. (2021b)).

Gene set enrichment analysis within the subset of differentially expressed genes identified “3D-structure” and “cell inner membrane” as overrepresented key words (FIG. 49). This suggests that genes associated with the cell membrane and 3D cell structure were overrepresented in the set of differentially expressed genes. This points to ace-K having a role in altering the bacterial cell membrane.

This study was repeated with saccharin and cyclamate, and the results are provided in Tables 3 and 4 below and in FIGS. 83 and 84 respectively.

TABLE 3

| Locustag | Gene | logFC | PValue |
|---|---|---|---|
| ABUW_0007 | | −1.0124 | 3.79E−05 |
| ABUW_0053 | | −1.14635 | 0.037825 |
| ABUW_0054 | | 1.154832 | 6.91E−06 |
| ABUW_0055 | | −1.5949 | 0.004024 |
| ABUW_0068 | | −1.269 | 0.002412 |
| ABUW_0118 | | 1.257218 | 0.000566 |
| ABUW_0119 | | 1.331992 | 2.79E−05 |
| ABUW_0128 | | −1.20877 | 0.004024 |
| ABUW_0158 | | 1.228519 | 0.001183 |
| ABUW_0178 | | 1.50999 | 8.46E−07 |
| ABUW_0179 | msuE | 1.483701 | 5.54E−06 |
| ABUW_0181 | | −2.02177 | 1.35E−06 |
| ABUW_0201 | gabP | 2.83556 | 9.14E−09 |
| ABUW_0203 | gabT | 3.116535 | 1.99E−10 |
| ABUW_0204 | gabD1 | 1.016736 | 9.06E−05 |
| ABUW_0224 | | 1.281932 | 0.000784 |
| ABUW_0248 | | −1.80115 | 2.07E−07 |
| ABUW_0259 | | −1.25083 | 0.008091 |
| ABUW_0260 | acr1 | −2.10937 | 8.74E−08 |
| ABUW_0266 | czcC | 1.00741 | 5.73E−06 |
| ABUW_0275 | | −1.11344 | 6.15E−05 |
| ABUW_0291 | comN | −1.17115 | 0.011929 |
| ABUW_0292 | comO | −1.17967 | 0.015941 |
| ABUW_0304 | | −2.33861 | 1.55E−06 |
| ABUW_0343 | | 1.368754 | 3.79E−06 |
| ABUW_0357 | | −1.07663 | 0.000503 |
| ABUW_0363 | | 1.074355 | 0.02078 |
| ABUW_0367 | glyQ | −1.06228 | 2.83E−05 |
| ABUW_0371 | | 1.673427 | 0.000161 |
| ABUW_0372 | | 1.459163 | 4.74E−05 |
| ABUW_0381 | | −2.98277 | 0.00016 |
| ABUW_0386 | mlaC | 1.288541 | 4.43E−08 |
| ABUW_0387 | mlaB | 1.472663 | 1.75E−07 |
| ABUW_0448 | | −1.84092 | 3.69E−06 |
| ABUW_0494 | rplM | −1.00689 | 0.003838 |
| ABUW_0566 | | 1.117624 | 0.024482 |
| ABUW_0583 | | 1.025985 | 0.000254 |
| ABUW_0603 | | 4.385368 | 1.50E−12 |
| ABUW_0607 | | 4.604278 | 6.77E−15 |
| ABUW_0628 | | −1.17177 | 0.028912 |
| ABUW_0641 | | −1.94821 | 2.81E−08 |
| ABUW_0642 | gcd | −2.05316 | 1.21E−08 |
| ABUW_0648 | | −1.12737 | 0.000246 |
| ABUW_0652 | lysP | −1.21207 | 7.17E−05 |
| ABUW_0653 | rsmC | −1.31948 | 2.93E−05 |
| ABUW_0654 | | −1.75459 | 2.93E−06 |
| ABUW_0655 | typA | −1.37429 | 4.26E−05 |
| ABUW_0684 | | −1.65367 | 3.00E−09 |
| ABUW_0687 | ecnB | −1.1971 | 0.006772 |
| ABUW_0703 | | −1.11168 | 1.17E−07 |
| ABUW_0720 | | −2.49775 | 4.44E−07 |
| ABUW_0725 | | 1.0897 | 1.29E−06 |
| ABUW_0763 | | 1.061555 | 0.004365 |
| ABUW_0768 | | 2.715359 | 0.004731 |
| ABUW_0789 | | 1.998296 | 0.00106 |
| ABUW_0790 | | 1.522368 | 2.28E−05 |
| ABUW_0807 | | 1.383309 | 0.000204 |
| ABUW_0809 | | 1.226238 | 0.012092 |
| ABUW_0823 | | −1.27832 | 9.30E−08 |
| ABUW_0865 | | −1.12524 | 0.000766 |
| ABUW_0881 | trpS | −1.1107 | 1.70E−05 |
| ABUW_0894 | carA | −1.21363 | 1.27E−06 |
| ABUW_0907 | folD | −1.00152 | 1.08E−05 |
| ABUW_0913 | pckG | −1.74792 | 7.75E−07 |
| ABUW_0918 | groL | 1.045625 | 0.000413 |
| ABUW_0922 | | 2.129261 | 9.18E−13 |
| ABUW_0926 | | −1.30097 | 8.78E−06 |
| ABUW_0964 | | 1.122556 | 0.001165 |
| ABUW_0969 | serC | −1.05087 | 3.54E−05 |
| ABUW_0981 | purM | −1.21536 | 8.13E−06 |
| ABUW_0990 | fbp1 | −1.48508 | 1.71E−06 |
| ABUW_1002 | purL | −1.24826 | 1.75E−06 |
| ABUW_1018 | cysW | 1.463253 | 2.41E−06 |
| ABUW_1019 | cysT | 3.407986 | 1.62E−08 |
| ABUW_1020 | | 4.398765 | 3.07E−07 |
| ABUW_1021 | cysP | 3.247179 | 3.54E−05 |
| ABUW_1040 | | 1.511991 | 0.000375 |
| ABUW_1043 | aspC | −1.26296 | 1.72E−06 |
| ABUW_1046 | | 1.108764 | 5.01E−08 |
| ABUW_1050 | gap | −1.34623 | 1.28E−06 |
| ABUW_1077 | | 4.647485 | 0.000883 |
| ABUW_1094 | | 1.440138 | 0.010042 |
| ABUW_1095 | | 2.787127 | 0.00229 |
| ABUW_1096 | | 3.566579 | 0.002604 |
| ABUW_1106 | | 1.977589 | 0.000274 |
| ABUW_1120 | | −1.11952 | 2.23E−06 |
| ABUW_1127 | dacC | −1.20304 | 6.50E−06 |
| ABUW_1159 | | 1.142013 | 0.008498 |
| ABUW_1165 | | −1.30789 | 0.000393 |
| ABUW_1172 | | 4.316579 | 0.034552 |
| ABUW_1183 | | 2.17066 | 0.023779 |
| ABUW_1224 | ahcY | −1.20253 | 6.13E−07 |
| ABUW_1226 | | −1.14335 | 2.27E−05 |
| ABUW_1227 | | −1.79573 | 0.000942 |
| ABUW_1244 | mrdB | −1.0186 | 4.44E−07 |
| ABUW_1248 | | −1.10402 | 2.06E−06 |
| ABUW_1280 | | 1.156379 | 0.000159 |
| ABUW_1283 | | 1.382851 | 7.62E−05 |
| ABUW_1284 | | 1.486346 | 0.000736 |
| ABUW_1286 | | −1.79052 | 0.019094 |
| ABUW_1292 | | 1.097871 | 1.59E−05 |
| ABUW_1301 | | 1.399241 | 0.006824 |
| ABUW_1308 | | 1.153749 | 0.008769 |
| ABUW_1310 | | 1.102836 | 0.020136 |
| ABUW_1321 | | −1.13895 | 0.000744 |
| ABUW_1330 | | 1.000324 | 0.002055 |
| ABUW_1333 | glyA | −1.26775 | 2.11E−06 |
| ABUW_1346 | | 1.009861 | 4.86E−05 |
| ABUW_1348 | | −1.26178 | 4.23E−06 |
| ABUW_1426 | | 1.027298 | 0.001894 |
| ABUW_1436 | | 1.148735 | 0.001415 |
| ABUW_1439 | | 1.386195 | 0.046579 |
| ABUW_1453 | | −1.11858 | 5.49E−05 |
| ABUW_1463 | | 1.619599 | 2.95E−06 |
| ABUW_1464 | mmsA2 | 1.231926 | 8.09E−06 |
| ABUW_1466 | | −1.71519 | 0.048771 |
| ABUW_1471 | | −1.64081 | 0.027277 |
| ABUW_1472 | | −1.21164 | 0.000631 |
| ABUW_1473 | | −1.58019 | 8.09E−05 |
| ABUW_1494 | | 1.343928 | 0.028098 |
| ABUW_1495 | | 1.734202 | 4.73E−05 |
| ABUW_1496 | proP | −1.00818 | 1.18E−06 |
| ABUW_1498 | | −1.24434 | 0.010612 |
| ABUW_1499 | | −1.66064 | 3.68E−07 |
| ABUW_1519 | | −1.63065 | 6.48E−06 |
| ABUW_1537 | gidA | −1.2371 | 2.29E−06 |
| ABUW_1563 | | 4.836137 | 1.99E−12 |
| ABUW_1564 | | 3.466339 | 5.54E−12 |
| ABUW_1567 | prfA | −1.08572 | 3.13E−07 |
| ABUW_1572 | | −3.69582 | 1.73E−07 |
| ABUW_1573 | | −3.61022 | 9.06E−07 |
| ABUW_1574 | | −3.23246 | 3.60E−06 |
| ABUW_1601 | | −2.13365 | 0.000983 |
| ABUW_1603 | | −1.2651 | 0.000121 |
| ABUW_1612 | | −1.35066 | 0.027377 |
| ABUW_1631 | | −2.41029 | 0.001385 |
| ABUW_1632 | | −1.9606 | 0.001807 |
| ABUW_1633 | | −1.61421 | 0.006141 |
| ABUW_1634 | | −2.18104 | 0.001959 |
| ABUW_1635 | | −1.82422 | 0.004145 |
| ABUW_1636 | | −1.80911 | 0.002902 |
| ABUW_1637 | | −1.63003 | 0.002878 |
| ABUW_1638 | | −1.03084 | 4.14E−05 |
| ABUW_1645 | | 1.315279 | 0.000339 |
| ABUW_1651 | | −4.05658 | 2.27E−05 |
| ABUW_1653 | | 1.14455 | 0.000373 |
| ABUW_1657 | | −1.07503 | 0.044076 |
| ABUW_1663 | | 1.091247 | 0.040186 |
| ABUW_1668 | | 1.373237 | 4.86E−08 |
| ABUW_1715 | | 1.487642 | 8.03E−10 |
| ABUW_1716 | | 1.201917 | 4.85E−05 |
| ABUW_1725 | | −1.13681 | 3.46E−05 |
| ABUW_1726 | | −1.01725 | 0.01159 |
| ABUW_1734 | | −1.11724 | 1.04E−06 |
| ABUW_1759 | | −1.65362 | 0.001175 |
| ABUW_1776 | | −1.10846 | 5.06E−05 |
| ABUW_1799 | | −1.16615 | 7.75E−08 |
| ABUW_1831 | pcaI1 | −1.05513 | 0.000912 |
| ABUW_1857 | | −2.7685 | 8.01E−05 |
| ABUW_1858 | | −2.56565 | 9.11E−05 |
| ABUW_1859 | | −1.97002 | 0.001871 |
| ABUW_1860 | | −1.98372 | 0.004541 |
| ABUW_1861 | | −1.73298 | 0.006332 |
| ABUW_1862 | | −1.54824 | 0.010678 |
| ABUW_1863 | | −1.29295 | 0.01872 |
| ABUW_1864 | | −1.13773 | 0.006398 |
| ABUW_1866 | feaR | 1.073823 | 0.002348 |
| ABUW_1886 | cpo | −1.31046 | 0.001586 |
| ABUW_1891 | | −1.13518 | 0.041768 |
| ABUW_1911 | | −1.20395 | 0.002319 |
| ABUW_1918 | | −1.74803 | 0.006651 |
| ABUW_1919 | | −1.3071 | 0.012441 |
| ABUW_1929 | | 1.930154 | 2.02E−07 |
| ABUW_1930 | | 2.324439 | 2.30E−09 |
| ABUW_1931 | | 2.906425 | 2.53E−10 |
| ABUW_1937 | | 1.017208 | 0.001744 |
| ABUW_1942 | | 1.434175 | 0.00361 |
| ABUW_1949 | | −1.66304 | 2.44E−06 |
| ABUW_1950 | | −1.63008 | 3.25E−07 |
| ABUW_1951 | | −1.21508 | 6.98E−05 |
| ABUW_1974 | adeA | −1.78249 | 2.60E−06 |
| ABUW_1975 | adeB | −1.02416 | 3.17E−05 |
| ABUW_2051 | | −1.15529 | 0.001257 |
| ABUW_2052 | | −1.59451 | 3.66E−05 |
| ABUW_2053 | | −2.4455 | 6.79E−08 |
| ABUW_2056 | | −1.0402 | 3.68E−06 |
| ABUW_2058 | | −1.40359 | 0.000687 |
| ABUW_2060 | | −1.65055 | 0.004875 |
| ABUW_2061 | | −1.21968 | 4.33E−05 |
| ABUW_2065 | | −1.27547 | 0.018483 |
| ABUW_2079 | modA | 1.06659 | 9.73E−05 |
| ABUW_2092 | bdhA | −1.50359 | 0.000213 |
| ABUW_2093 | | −2.16485 | 1.20E−06 |
| ABUW_2096 | atoD | −2.08558 | 0.000111 |
| ABUW_2097 | atoA | −2.16911 | 0.00019 |
| ABUW_2098 | atoE | −1.81919 | 0.000788 |
| ABUW_2099 | | −2.03638 | 0.000243 |
| ABUW_2104 | aspA | −1.27718 | 1.75E−05 |
| ABUW_2112 | | 1.105269 | 0.000916 |
| ABUW_2113 | | 1.749893 | 7.29E−08 |
| ABUW_2122 | | −1.30192 | 0.000393 |
| ABUW_2127 | budC | 1.598127 | 1.23E−05 |
| ABUW_2128 | lpdA2 | 1.806852 | 0.000342 |
| ABUW_2129 | acoC | 1.748709 | 0.000254 |
| ABUW_2130 | acoB | 1.719081 | 0.000485 |
| ABUW_2131 | acoA | 1.580792 | 0.000608 |
| ABUW_2137 | prmB | −1.52232 | 1.40E−06 |
| ABUW_2145 | | −1.28228 | 0.005354 |
| ABUW_2169 | | 1.250465 | 0.000202 |
| ABUW_2179 | | 1.136741 | 0.003464 |
| ABUW_2184 | | 1.201843 | 0.002412 |
| ABUW_2185 | | 1.104346 | 0.000323 |
| ABUW_2186 | | 1.040971 | 0.000314 |
| ABUW_2187 | | 1.270271 | 0.000654 |
| ABUW_2216 | rpsT | −1.29813 | 0.00025 |
| ABUW_2275 | | −1.03597 | 0.003206 |
| ABUW_2276 | | 1.244193 | 0.000525 |
| ABUW_2286 | | 1.834991 | 0.00038 |
| ABUW_2287 | putA | 4.279175 | 0.001019 |
| ABUW_2288 | | 1.122801 | 0.010121 |
| ABUW_2289 | trmA | −1.01158 | 2.39E−05 |
| ABUW_2290 | | −1.09762 | 0.001721 |
| ABUW_2293 | | −1.03084 | 0.000268 |
| ABUW_2310 | | −1.78454 | 1.98E−05 |
| ABUW_2311 | | −1.0058 | 0.006929 |
| ABUW_2314 | | 1.240754 | 0.00118 |
| ABUW_2316 | | −2.05795 | 5.51E−05 |
| ABUW_2317 | | −1.07649 | 0.016404 |
| ABUW_2321 | | −2.15127 | 0.005406 |
| ABUW_2326 | | −1.9049 | 1.34E−05 |
| ABUW_2335 | | 1.814365 | 1.46E−06 |
| ABUW_2336 | | 2.06068 | 1.56E−08 |
| ABUW_2337 | | 1.565051 | 1.79E−05 |
| ABUW_2342 | ychF | −1.88692 | 1.30E−06 |
| ABUW_2349 | | −1.13726 | 4.66E−07 |
| ABUW_2358 | | −1.30914 | 2.30E−06 |
| ABUW_2371 | arsC1 | 1.2316 | 0.001076 |
| ABUW_2378 | hyu | 2.744304 | 8.16E−06 |
| ABUW_2379 | tauD | 2.750083 | 8.10E−08 |
| ABUW_2380 | tauC | 3.527504 | 8.63E−09 |
| ABUW_2381 | tauB | 3.838485 | 1.88E−07 |
| ABUW_2382 | tauA | 2.916346 | 7.83E−05 |
| ABUW_2385 | | 1.055764 | 2.03E−05 |
| ABUW_2389 | cioA | −1.5979 | 0.005718 |
| ABUW_2390 | cioB | −1.53785 | 0.005848 |
| ABUW_2391 | | −1.25459 | 0.006313 |
| ABUW_2403 | | −1.03261 | 2.19E−06 |
| ABUW_2414 | | 1.692418 | 0.002323 |
| ABUW_2415 | srpH | 1.527005 | 4.25E−05 |
| ABUW_2416 | | 1.395863 | 1.88E−06 |
| ABUW_2421 | | 1.203662 | 0.000781 |
| ABUW_2422 | | 2.838883 | 2.88E−07 |
| ABUW_2423 | | 3.258313 | 3.27E−07 |
| ABUW_2424 | | 2.200242 | 2.22E−05 |
| ABUW_2430 | | 1.106832 | 0.000784 |
| ABUW_2433 | | −3.982 | 0.005647 |
| ABUW_2435 | | −2.49477 | 0.010038 |
| ABUW_2436 | katE | −2.03817 | 0.008531 |
| ABUW_2437 | | −1.77464 | 0.027798 |
| ABUW_2439 | | −3.12153 | 0.007632 |
| ABUW_2440 | | −2.53223 | 0.027463 |
| ABUW_2442 | | −1.74249 | 0.004415 |
| ABUW_2443 | | −2.12455 | 0.013583 |
| ABUW_2448 | | −1.24858 | 0.002091 |
| ABUW_2449 | | −1.0397 | 0.011641 |
| ABUW_2450 | | −1.07684 | 0.011051 |
| ABUW_2452 | | −1.20039 | 0.001013 |
| ABUW_2453 | | −1.35503 | 0.001271 |
| ABUW_2454 | mgh | −1.36264 | 0.001944 |
| ABUW_2455 | | −1.47249 | 0.000623 |
| ABUW_2456 | | −1.33885 | 0.000903 |
| ABUW_2458 | | −1.54111 | 0.003144 |
| ABUW_2489 | | −1.0686 | 3.88E−05 |
| ABUW_2491 | | 1.128276 | 0.007547 |
| ABUW_2503 | | −1.06202 | 0.011285 |
| ABUW_2504 | | −1.33272 | 0.005046 |
| ABUW_2546 | | 1.102767 | 0.00406 |
| ABUW_2553 | | −1.97586 | 0.000964 |
| ABUW_2554 | | −1.66589 | 5.82E−06 |
| ABUW_2557 | | −1.99694 | 5.07E−08 |
| ABUW_2588 | | 1.66839 | 0.040801 |
| ABUW_2594 | | −2.33064 | 0.011428 |
| ABUW_2602 | | −1.14016 | 4.00E−05 |
| ABUW_2603 | bccA | −4.78629 | 7.64E−08 |
| ABUW_2604 | | −4.90488 | 3.67E−08 |
| ABUW_2605 | | −5.55089 | 3.11E−08 |
| ABUW_2606 | | −5.51171 | 5.63E−08 |
| ABUW_2607 | | −6.40407 | 1.75E−08 |
| ABUW_2614 | | 1.974413 | 0.00461 |
| ABUW_2615 | | 1.353638 | 0.000134 |
| ABUW_2621 | | −2.14282 | 8.57E−06 |
| ABUW_2627 | | −2.38195 | 2.57E−08 |
| ABUW_2658 | | −1.73034 | 8.83E−08 |
| ABUW_2673 | | −1.61268 | 0.005154 |
| ABUW_2678 | | −1.6258 | 0.002756 |
| ABUW_2679 | | −3.12661 | 0.004034 |
| ABUW_2681 | | −1.14812 | 1.32E−05 |
| ABUW_2684 | | −1.55216 | 0.010758 |
| ABUW_2685 | | −1.9312 | 0.001583 |
| ABUW_2686 | | −1.49177 | 0.008535 |
| ABUW_2700 | | −1.3465 | 1.39E−05 |
| ABUW_2701 | | −2.04038 | 2.29E−08 |
| ABUW_2703 | | −1.90135 | 0.001619 |
| ABUW_2723 | ahpF2 | −1.29522 | 0.000266 |
| ABUW_2727 | trmB | −2.04126 | 5.40E−07 |
| ABUW_2755 | | −1.08025 | 1.11E−07 |
| ABUW_2783 | | 1.478796 | 1.74E−05 |
| ABUW_2813 | | 1.771035 | 0.004517 |
| ABUW_2814 | | 1.22519 | 0.000373 |
| ABUW_2815 | | 1.013844 | 5.06E−06 |
| ABUW_2818 | | 1.446617 | 0.005457 |
| ABUW_2823 | argG | −1.11595 | 6.38E−05 |
| ABUW_2829 | | 1.014097 | 0.002288 |
| ABUW_2870 | | −1.1263 | 2.49E−05 |
| ABUW_2887 | | −1.56301 | 0.000396 |
| ABUW_2899 | lysS | −1.29164 | 1.50E−06 |
| ABUW_2933 | | −1.32103 | 0.000146 |
| ABUW_2938 | | 1.894141 | 0.028147 |
| ABUW_2939 | | −1.12692 | 1.04E−05 |
| ABUW_2940 | | 1.63195 | 6.68E−06 |
| ABUW_2941 | | 1.460392 | 4.57E−06 |
| ABUW_2942 | | 1.786817 | 9.07E−08 |
| ABUW_2943 | | 1.695844 | 1.55E−06 |
| ABUW_2944 | | 1.492233 | 6.55E−07 |
| ABUW_2945 | | 1.763622 | 2.20E−07 |
| ABUW_2946 | | 1.37974 | 1.21E−06 |
| ABUW_2947 | | 1.359446 | 7.76E−07 |
| ABUW_2948 | | 2.438401 | 1.13E−07 |
| ABUW_2949 | | 2.904062 | 1.09E−09 |
| ABUW_2950 | | 3.568554 | 6.99E−10 |
| ABUW_2951 | | 3.988474 | 1.59E−12 |
| ABUW_2952 | | 4.132519 | 6.61E−10 |
| ABUW_2953 | | 3.965604 | 6.25E−09 |
| ABUW_2954 | | 2.045373 | 2.94E−09 |
| ABUW_2964 | | 1.207495 | 2.44E−07 |
| ABUW_2965 | | 2.120546 | 4.81E−09 |
| ABUW_3037 | | 1.1781 | 0.038401 |
| ABUW_3097 | prs | −1.0319 | 0.000276 |
| ABUW_3100 | panD | −1.02368 | 1.72E−05 |
| ABUW_3122 | otsB | −1.82281 | 0.041706 |
| ABUW_3136 | dusC | −1.07482 | 0.000312 |
| ABUW_3150 | | −1.17104 | 4.14E−06 |
| ABUW_3157 | | −1.34569 | 0.002478 |
| ABUW_3182 | | −1.00737 | 0.000437 |
| ABUW_3196 | | −1.02536 | 1.34E−06 |
| ABUW_3197 | metE | −3.13307 | 1.98E−12 |
| ABUW_3198 | | −3.01915 | 4.20E−11 |
| ABUW_3204 | | −1.09438 | 6.70E−05 |
| ABUW_3214 | gcdH | −1.31486 | 1.40E−05 |
| ABUW_3253 | rpoN | 1.036779 | 3.10E−05 |
| ABUW_3305 | cysM | −4.11773 | 0.009888 |
| ABUW_3312 | pntB | −2.42632 | 1.58E−09 |
| ABUW_3313 | pntA2 | −2.64463 | 1.92E−09 |
| ABUW_3314 | pntA1 | −3.24162 | 5.23E−10 |
| ABUW_3320 | copB | −1.05152 | 0.008025 |
| ABUW_3321 | copA | −1.05879 | 0.015582 |
| ABUW_3322 | | −1.46907 | 0.018474 |
| ABUW_3325 | actP1 | −1.80659 | 0.000907 |
| ABUW_3332 | | 1.066036 | 0.008471 |
| ABUW_3343 | | −2.03737 | 5.91E−10 |
| ABUW_3351 | | −2.06804 | 4.65E−07 |
| ABUW_3352 | | −1.09432 | 3.20E−05 |
| ABUW_3362 | | 1.441725 | 8.61E−09 |
| ABUW_3363 | macB | 1.343168 | 5.04E−09 |
| ABUW_3364 | | 1.34358 | 1.31E−08 |
| ABUW_3383 | | −1.80169 | 2.68E−06 |
| ABUW_3384 | | −1.12238 | 6.36E−06 |
| ABUW_3398 | | 1.851143 | 5.53E−07 |
| ABUW_3414 | | 1.001031 | 1.35E−05 |
| ABUW_3449 | dctA | −1.62407 | 8.01E−06 |
| ABUW_3457 | infA | −1.473 | 6.67E−05 |
| ABUW_3469 | katG | 1.01412 | 2.27E−05 |
| ABUW_3484 | | 1.008253 | 0.003199 |
| ABUW_3495 | | −1.12036 | 0.000302 |
| ABUW_3543 | | −1.54171 | 0.000147 |
| ABUW_3560 | | 1.02684 | 5.22E−06 |
| ABUW_3561 | | 1.418979 | 8.28E−05 |
| ABUW_3562 | | 1.569805 | 9.13E−09 |
| ABUW_3563 | | 1.448155 | 2.42E−09 |
| ABUW_3575 | | −2.11849 | 0.000402 |
| ABUW_3584 | | −1.2638 | 1.81E−05 |
| ABUW_3588 | | 1.224516 | 4.23E−05 |
| ABUW_3596 | secE | −1.01368 | 3.74E−05 |
| ABUW_3598 | tuf2 | −1.06387 | 0.001943 |
| ABUW_3622 | | −1.23736 | 0.000536 |
| ABUW_3718 | | −2.89117 | 1.56E−09 |
| ABUW_3719 | | −3.65248 | 1.02E−08 |
| ABUW_3720 | | −2.9169 | 8.29E−09 |
| ABUW_3730 | | 1.279445 | 4.31E−06 |
| ABUW_3739 | atpI | −1.07197 | 0.00018 |
| ABUW_3759 | | −1.56158 | 2.20E−05 |
| ABUW_3771 | | 1.13479 | 0.000414 |
| ABUW_3776 | cepI | 1.307123 | 0.000134 |
| ABUW_3777 | | 1.008751 | 0.002023 |
| ABUW_3778 | | 1.049388 | 0.00097 |
| ABUW_3779 | | 1.167161 | 0.002457 |
| ABUW_3780 | | 1.403476 | 0.000307 |
| ABUW_3781 | | 1.084284 | 0.009515 |
| ABUW_3788 | dadX | 1.59151 | 4.70E−08 |
| ABUW_3790 | lrp | 1.731256 | 1.98E−07 |
| ABUW_3791 | | 1.010083 | 0.000355 |
| ABUW_3798 | | −1.26387 | 6.99E−07 |
| ABUW_3801 | | 1.800123 | 0.000401 |
| ABUW_3806 | acnD | −1.10812 | 8.34E−05 |
| ABUW_3807 | prpC | −1.35278 | 6.90E−06 |
| ABUW_3808 | prpB | −1.34592 | 3.10E−05 |
| ABUW_3809 | | −1.08041 | 0.000157 |
| ABUW_3839 | | 2.210989 | 1.80E−06 |
| ABUW_3842 | | −1.59784 | 0.001705 |
| ABUW_3843 | | −2.66882 | 0.00053 |
| ABUW_3851 | | 2.786902 | 1.88E−10 |
| ABUW_3853 | ssuA1 | 1.6114 | 0.001115 |
| ABUW_3854 | ssuA2 | 1.604256 | 1.18E−06 |
| ABUW_3875 | | 1.938346 | 6.42E−08 |
| ABUW_3884 | | 1.326699 | 0.007792 |
| ABUW_3898 | | −1.43746 | 0.012502 |
| ABUW_3904 | | −1.06005 | 0.001501 |
| ABUW_3905 | rnpA | −1.13408 | 0.001272 |
| ABUW_3906 | rpmH | −1.12849 | 0.00159 |
| ABUW_4026 | | 1.273088 | 0.000111 |
| ABUW_4027 | | 1.484738 | 0.003057 |
| ABUW_4032 | | 1.199946 | 0.022943 |
| ABUW_4066 | | 1.088486 | 0.008153 |
| ABUW_4071 | | 1.393717 | 0.007816 |
| ABUW_4074 | | 1.460329 | 0.004152 |
| ABUW_4083 | | 1.897799 | 0.00014 |
| ABUW_4091 | | 1.432754 | 0.003176 |
| ABUW_4123 | | 1.392903 | 0.008006 |
| ABUW_5013 | | 1.52695 | 0.000567 |

TABLE 4

| Locustag | Gene | logFC | PValue |
|---|---|---|---|
| ABUW_0030 | | −1.00156 | 9.50E−06 |
| ABUW_0053 | | −1.5644 | 0.008096 |
| ABUW_0055 | | −1.55429 | 0.004743 |
| ABUW_0077 | hutU | 1.040407 | 6.43E−06 |
| ABUW_0178 | | 1.475689 | 1.16E−06 |
| ABUW_0179 | msuE | 1.97178 | 2.06E−07 |
| ABUW_0181 | | −1.82429 | 4.12E−06 |
| ABUW_0187 | | 1.13682 | 0.033746 |
| ABUW_0201 | gabP | 1.06313 | 0.000355 |
| ABUW_0203 | gabT | 1.001989 | 8.68E−05 |
| ABUW_0223 | | 1.25397 | 0.001276 |
| ABUW_0248 | | −1.09781 | 4.76E−05 |
| ABUW_0255 | | 1.028739 | 5.32E−05 |
| ABUW_0280 | sbp | 1.853653 | 4.29E−05 |
| ABUW_0291 | comN | −1.11643 | 0.017046 |
| ABUW_0304 | | −3.29875 | 3.93E−08 |
| ABUW_0314 | pilV | −1.07274 | 0.034789 |
| ABUW_0353 | astD | 1.05455 | 1.60E−05 |
| ABUW_0354 | astB | 1.249713 | 2.84E−06 |
| ABUW_0355 | astE | 1.283219 | 7.38E−07 |
| ABUW_0356 | | 1.007692 | 9.84E−07 |
| ABUW_0381 | | −1.4664 | 0.017585 |
| ABUW_0550 | | −1.19334 | 9.55E−06 |
| ABUW_0585 | | −1.02792 | 5.88E−05 |
| ABUW_0603 | | 1.981152 | 4.15E−08 |
| ABUW_0607 | | 2.814094 | 2.79E−12 |
| ABUW_0641 | | −1.23995 | 5.37E−06 |
| ABUW_0642 | gcd | −1.29511 | 2.75E−06 |
| ABUW_0648 | | −1.01219 | 0.000633 |
| ABUW_0653 | rsmC | −1.0057 | 0.00039 |
| ABUW_0654 | | −1.20135 | 0.00014 |
| ABUW_0684 | | −1.63632 | 3.73E−09 |
| ABUW_0687 | ecnB | −1.05838 | 0.013699 |
| ABUW_0720 | | −1.63045 | 3.70E−05 |
| ABUW_0826 | | 1.301193 | 1.60E−06 |
| ABUW_0894 | carA | −1.08941 | 4.31E−06 |
| ABUW_0913 | pckG | −1.21145 | 4.05E−05 |
| ABUW_0917 | groS | 1.418528 | 8.82E−05 |
| ABUW_0918 | groL | 1.867623 | 1.19E−06 |
| ABUW_0922 | | 1.009874 | 1.59E−08 |
| ABUW_1018 | cysW | 1.604541 | 8.57E−07 |
| ABUW_1019 | cysT | 3.674953 | 6.84E−09 |
| ABUW_1020 | | 4.528654 | 2.33E−07 |
| ABUW_1021 | cysP | 4.318399 | 2.89E−06 |
| ABUW_1064 | | −1.00631 | 0.017664 |
| ABUW_1096 | | 2.572191 | 0.041596 |
| ABUW_1165 | | −1.52794 | 0.000101 |
| ABUW_1183 | | 2.070437 | 0.037202 |
| ABUW_1226 | | −1.3365 | 4.43E−06 |
| ABUW_1227 | | −1.98639 | 0.000432 |
| ABUW_1233 | | 1.580377 | 1.16E−06 |
| ABUW_1259 | | −1.15207 | 0.00603 |
| ABUW_1280 | | 1.261503 | 7.44E−05 |
| ABUW_1286 | | −1.57878 | 0.034095 |
| ABUW_1287 | | −1.30185 | 0.023109 |
| ABUW_1302 | | −1.21082 | 0.017848 |
| ABUW_1347 | | −1.51573 | 1.34E−06 |
| ABUW_1379 | | −1.0804 | 0.001789 |
| ABUW_1431 | | −1.01504 | 0.000124 |
| ABUW_1462 | | 1.212811 | 9.54E−06 |
| ABUW_1466 | | −2.03127 | 0.024343 |
| ABUW_1468 | | −1.5348 | 0.046394 |
| ABUW_1469 | | −1.57896 | 0.048037 |
| ABUW_1470 | | −1.56009 | 0.044832 |
| ABUW_1471 | | −2.19685 | 0.006229 |
| ABUW_1498 | | −1.55722 | 0.003981 |
| ABUW_1519 | | −1.775 | 2.71E−06 |
| ABUW_1536 | | −1.34492 | 0.009573 |
| ABUW_1542 | | −1.01457 | 0.004861 |
| ABUW_1563 | | 2.749164 | 1.33E−09 |
| ABUW_1564 | | 1.878269 | 9.97E−09 |
| ABUW_1570 | | 1.062024 | 0.001033 |
| ABUW_1572 | | −3.05554 | 1.11E−06 |
| ABUW_1573 | | −3.12502 | 3.52E−06 |
| ABUW_1574 | | −3.07334 | 5.85E−06 |
| ABUW_1601 | | −1.77683 | 0.003582 |
| ABUW_1602 | rsuA | −1.1716 | 0.001454 |
| ABUW_1603 | | −1.09785 | 0.00044 |
| ABUW_1629 | | −1.66065 | 0.000529 |
| ABUW_1631 | | −2.44699 | 0.001247 |
| ABUW_1632 | | −1.74592 | 0.003907 |
| ABUW_1633 | | −1.6074 | 0.006303 |
| ABUW_1634 | | −1.97784 | 0.003716 |
| ABUW_1635 | | −1.61605 | 0.008612 |
| ABUW_1636 | | −1.59785 | 0.006391 |
| ABUW_1637 | | −1.56337 | 0.003848 |
| ABUW_1651 | | −2.64187 | 0.000631 |
| ABUW_1653 | | 1.045009 | 0.000863 |
| ABUW_1657 | | −1.51474 | 0.008479 |
| ABUW_1659 | | −1.56446 | 0.038172 |
| ABUW_1665 | | −1.28057 | 0.00122 |
| ABUW_1668 | | 1.037952 | 1.49E−06 |
| ABUW_1726 | | −1.10947 | 0.006973 |
| ABUW_1759 | | −1.13538 | 0.013024 |
| ABUW_1787 | | −1.313 | 0.001142 |
| ABUW_1857 | | −1.23673 | 0.019557 |
| ABUW_1866 | feaR | 1.378016 | 0.000319 |
| ABUW_1886 | cpo | −1.60521 | 0.000314 |
| ABUW_1891 | | −1.67734 | 0.005858 |
| ABUW_1911 | | −1.22739 | 0.002876 |
| ABUW_1918 | | −2.25757 | 0.001805 |
| ABUW_1931 | | −1.11402 | 3.99E−05 |
| ABUW_1949 | | −1.53428 | 6.77E−06 |
| ABUW_1950 | | −2.17724 | 1.57E−08 |
| ABUW_1951 | | −1.68527 | 3.41E−06 |
| ABUW_1952 | | −1.04691 | 3.75E−06 |
| ABUW_1953 | | −1.30051 | 1.46E−05 |
| ABUW_1974 | adeA | −1.15207 | 0.00021 |
| ABUW_2052 | | −1.28281 | 0.000285 |
| ABUW_2053 | | −1.76367 | 2.74E−06 |
| ABUW_2054 | | −1.06553 | 0.000207 |
| ABUW_2056 | | −1.00574 | 5.71E−06 |
| ABUW_2058 | | −1.52977 | 0.000336 |
| ABUW_2060 | | −1.71007 | 0.003927 |
| ABUW_2065 | | −1.67314 | 0.003958 |
| ABUW_2092 | bdhA | −1.97957 | 1.71E−05 |
| ABUW_2093 | | −2.58548 | 1.84E−07 |
| ABUW_2096 | atoD | −2.70861 | 1.07E−05 |
| ABUW_2097 | atoA | −2.68858 | 3.03E−05 |
| ABUW_2098 | atoE | −2.05197 | 0.000312 |
| ABUW_2099 | | −2.44896 | 4.99E−05 |
| ABUW_2111 | | 1.203568 | 0.000162 |
| ABUW_2112 | | 1.034402 | 0.00178 |
| ABUW_2113 | | 1.78125 | 6.09E−08 |
| ABUW_2114 | | 1.808884 | 0.000364 |
| ABUW_2121 | | −1.83303 | 1.37E−05 |
| ABUW_2122 | | −2.6798 | 3.28E−07 |
| ABUW_2123 | | −2.61923 | 2.29E−08 |
| ABUW_2127 | budC | 1.172623 | 0.000251 |
| ABUW_2128 | IpdA2 | 1.264184 | 0.004716 |
| ABUW_2129 | acoC | 1.360054 | 0.001861 |
| ABUW_2130 | acoB | 1.442344 | 0.001854 |
| ABUW_2131 | acoA | 1.49869 | 0.000925 |
| ABUW_2145 | | −1.54727 | 0.00192 |
| ABUW_2149 | | −1.28213 | 0.000208 |
| ABUW_2169 | | 1.38615 | 7.81E−05 |
| ABUW_2287 | putA | 4.43535 | 0.000815 |
| ABUW_2288 | | 1.732822 | 0.000425 |
| ABUW_2290 | | −1.54203 | 0.000111 |
| ABUW_2293 | | −1.05126 | 0.000225 |
| ABUW_2310 | | −2.30168 | 2.06E−06 |
| ABUW_2311 | | −1.71217 | 0.000178 |
| ABUW_2320 | | −1.02636 | 0.004989 |
| ABUW_2321 | | −2.43717 | 0.003026 |
| ABUW_2326 | | −1.20019 | 0.000943 |
| ABUW_2335 | | 2.205289 | 1.51E−07 |
| ABUW_2336 | | 1.929268 | 3.67E−08 |
| ABUW_2337 | | 1.802541 | 4.04E−06 |
| ABUW_2342 | ychF | −1.02599 | 0.00055 |
| ABUW_2378 | hyu | 2.721847 | 9.38E−06 |
| ABUW_2379 | tauD | 3.033783 | 2.72E−08 |
| ABUW_2380 | tauC | 3.206211 | 2.45E−08 |
| ABUW_2381 | tauB | 3.712308 | 2.61E−07 |
| ABUW_2382 | tauA | 3.898207 | 6.39E−06 |
| ABUW_2385 | | 1.021647 | 3.09E−05 |
| ABUW_2386 | | 1.151501 | 8.06E−05 |
| ABUW_2389 | cioA | −2.00419 | 0.001276 |
| ABUW_2390 | cioB | −2.0841 | 0.000738 |
| ABUW_2391 | | −1.86626 | 0.00039 |
| ABUW_2403 | | −1.15611 | 6.87E−07 |
| ABUW_2414 | | 2.074947 | 0.000473 |
| ABUW_2415 | srpH | 1.259305 | 0.000287 |
| ABUW_2416 | | 1.1597 | 1.59E−05 |
| ABUW_2418 | | −1.30793 | 3.07E−06 |
| ABUW_2421 | | 1.729324 | 2.92E−05 |
| ABUW_2422 | | 2.715752 | 4.89E−07 |
| ABUW_2423 | | 3.073992 | 6.56E−07 |
| ABUW_2424 | | 2.515662 | 5.63E−06 |
| ABUW_2433 | | −4.87964 | 0.001893 |
| ABUW_2434 | | −1.6416 | 0.043174 |
| ABUW_2435 | | −2.60117 | 0.008055 |
| ABUW_2436 | katE | −2.51152 | 0.002437 |
| ABUW_2437 | | −1.88643 | 0.021099 |
| ABUW_2439 | | −3.74166 | 0.002826 |
| ABUW_2440 | | −3.29728 | 0.008034 |
| ABUW_2441 | | −1.17712 | 0.012655 |
| ABUW_2442 | | −1.12635 | 0.041772 |
| ABUW_2443 | | −2.24821 | 0.010345 |
| ABUW_2449 | | −1.21926 | 0.004552 |
| ABUW_2450 | | −1.18322 | 0.00636 |
| ABUW_2451 | | −1.62822 | 3.37E−05 |
| ABUW_2452 | | −1.38587 | 0.000309 |
| ABUW_2453 | | −1.47382 | 0.000658 |
| ABUW_2454 | mgh | −1.44031 | 0.001294 |
| ABUW_2455 | | −1.55856 | 0.00039 |
| ABUW_2456 | | −1.62204 | 0.000181 |
| ABUW_2458 | | −2.03469 | 0.000411 |
| ABUW_2503 | | −1.29597 | 0.003363 |
| ABUW_2504 | | −1.34712 | 0.00472 |
| ABUW_2553 | | −1.31822 | 0.013368 |
| ABUW_2554 | | −1.38618 | 3.92E−05 |
| ABUW_2557 | | −2.47134 | 4.14E−09 |
| ABUW_2589 | | −1.11037 | 0.007265 |
| ABUW_2590 | | 1.312661 | 0.000115 |
| ABUW_2594 | | −2.56791 | 0.006828 |
| ABUW_2603 | bccA | −4.38333 | 1.73E−07 |
| ABUW_2604 | | −4.40024 | 1.01E−07 |
| ABUW_2605 | | −4.48442 | 2.08E−07 |
| ABUW_2606 | | −4.69701 | 2.32E−07 |
| ABUW_2607 | | −5.60859 | 5.50E−08 |
| ABUW_2614 | | 1.819372 | 0.01039 |
| ABUW_2621 | | −1.3828 | 0.000503 |
| ABUW_2627 | | −1.99152 | 2.27E−07 |
| ABUW_2630 | | 1.136093 | 0.025578 |
| ABUW_2655 | | 1.14529 | 0.00014 |
| ABUW_2658 | | −1.24648 | 3.97E−06 |
| ABUW_2665 | | 1.053243 | 0.001621 |
| ABUW_2666 | | 1.21583 | 0.007697 |
| ABUW_2670 | | −1.21767 | 0.012224 |
| ABUW_2673 | | −1.04033 | 0.046901 |
| ABUW_2678 | | −2.24208 | 0.000267 |
| ABUW_2679 | | −3.72632 | 0.001425 |
| ABUW_2683 | | −1.53513 | 0.012823 |
| ABUW_2684 | | −1.58994 | 0.009529 |
| ABUW_2685 | | −1.55451 | 0.006784 |
| ABUW_2700 | | −1.24696 | 3.30E−05 |
| ABUW_2701 | | −1.64721 | 3.04E−07 |
| ABUW_2703 | | −1.99356 | 0.001168 |
| ABUW_2704 | | −1.36837 | 0.018689 |
| ABUW_2708 | | 1.263417 | 0.005859 |
| ABUW_2727 | trmB | −1.21287 | 0.000126 |
| ABUW_2756 | | −1.5753 | 4.87E−06 |
| ABUW_2782 | hcaE | 1.192799 | 4.77E−05 |
| ABUW_2783 | | 1.952644 | 7.28E−07 |
| ABUW_2784 | | 1.628766 | 2.84E−05 |
| ABUW_2785 | hcaG | 1.223582 | 0.000107 |
| ABUW_2800 | | 1.084443 | 0.000364 |
| ABUW_2813 | | 1.605633 | 0.011327 |
| ABUW_2887 | | −1.47805 | 0.000628 |
| ABUW_2892 | citN | 1.066922 | 1.45E−07 |
| ABUW_2896 | cysD | 1.072322 | 2.69E−05 |
| ABUW_2922 | | −1.57361 | 1.22E−06 |
| ABUW_2933 | | −1.34311 | 0.000126 |
| ABUW_2942 | | 1.096322 | 2.60E−05 |
| ABUW_2964 | | 1.039576 | 1.52E−06 |
| ABUW_2965 | | 2.74642 | 2.04E−10 |
| ABUW_2984 | | −1.0541 | 5.58E−06 |
| ABUW_3037 | | 1.376907 | 0.019083 |
| ABUW_3123 | otsA | −1.89522 | 0.039502 |
| ABUW_3124 | | −1.44856 | 0.01017 |
| ABUW_3157 | | −1.7295 | 0.00036 |
| ABUW_3158 | | −1.26632 | 0.004871 |
| ABUW_3196 | | −1.95407 | 4.91E−10 |
| ABUW_3197 | metE | −3.2359 | 1.35E−12 |
| ABUW_3198 | | −2.83434 | 9.01E−11 |
| ABUW_3214 | gcdH | −1.05042 | 0.000133 |
| ABUW_3305 | cysM | −3.56447 | 0.018821 |
| ABUW_3312 | pntB | −2.39935 | 1.86E−09 |
| ABUW_3313 | pntA2 | −2.65872 | 2.00E−09 |
| ABUW_3314 | pntA1 | −3.11048 | 8.60E−10 |
| ABUW_3320 | copB | −1.42666 | 0.001028 |
| ABUW_3321 | copA | −1.25668 | 0.005897 |
| ABUW_3322 | | −1.75757 | 0.007146 |
| ABUW_3325 | actP1 | −2.40753 | 8.83E−05 |
| ABUW_3343 | | −1.00057 | 3.33E−06 |
| ABUW_3351 | | −1.75285 | 2.95E−06 |
| ABUW_3383 | | −1.56044 | 1.27E−05 |
| ABUW_3390 | gapN | 1.080235 | 1.13E−06 |
| ABUW_3398 | | 1.196384 | 6.48E−05 |
| ABUW_3449 | dctA | −1.01348 | 0.0007 |
| ABUW_3473 | | −1.03019 | 9.81E−05 |
| ABUW_3482 | | 1.060454 | 0.000175 |
| ABUW_3575 | | −2.22672 | 0.000274 |
| ABUW_3591 | rpIL | 1.081223 | 0.00134 |
| ABUW_3622 | | −1.54279 | 9.36E−05 |
| ABUW_3718 | | −3.08089 | 8.42E−10 |
| ABUW_3719 | | −3.7598 | 7.54E−09 |
| ABUW_3720 | | −3.18621 | 3.10E−09 |
| ABUW_3739 | atpI | −1.07513 | 0.000178 |
| ABUW_3786 | cycA2 | 1.326594 | 6.68E−08 |
| ABUW_3788 | dadX | 1.050588 | 6.06E−06 |
| ABUW_3790 | Irp | 1.728576 | 2.10E−07 |
| ABUW_3798 | | −1.05724 | 5.83E−06 |
| ABUW_3814 | IIdP | 1.048801 | 0.008855 |
| ABUW_3839 | | 1.234917 | 0.000691 |
| ABUW_3842 | | −1.19056 | 0.011154 |
| ABUW_3843 | | −1.99358 | 0.003912 |
| ABUW_3851 | | 1.07095 | 1.95E−05 |
| ABUW_3853 | ssuA1 | 2.835062 | 5.30E−06 |
| ABUW_3854 | ssuA2 | 1.828335 | 2.62E−07 |
| ABUW_3855 | ssuD | 1.312561 | 7.78E−06 |
| ABUW_3856 | ssuC | 1.314393 | 3.82E−05 |
| ABUW_3875 | | 2.272276 | 9.55E−09 |
| ABUW_3879 | dnak | 1.240247 | 1.59E−05 |
| ABUW_3898 | | −1.76001 | 0.003814 |
| ABUW_4066 | | 1.271856 | 0.003129 |
| ABUW_4074 | | 1.991518 | 0.000334 |
| ABUW_4096 | | −1.00885 | 0.000311 |

In the presence of saccharin, 429 genes were significantly differently regulated (Table 3). Gene set enrichment analysis identified Gene Ontology pathways associated with cell adhesion, motility and outer membrane proteins being enriched. In the presence of cyclamate, 288 genes were significantly differently expressed (Table 4). Gene set enrichment analysis identified also Gene Ontology pathways cell adhesion, pilus and pilus organisation as well as transmembrane transports being enriched.

### Example 8: Motility Effect

Motility is a central facet of bacterial virulence and facilitates bacterial dissemination to the blood stream or other sites within an infected host. The transcriptomic data suggested that ace-K could inhibit the expression of genes associated with A. baumannii twitching motility. To validate the gene expression data, we performed twitching assays at a range of different concentrations. In agreement with the gene expression/transcriptomic data, a significant reduction in bacterial twitching motility to as low as 0.33% ace-K was observed. FIG. 50 illustrates the results, in which the data are derived from three biological replicates.

These results further support the capacity of ace-K to have an anti-virulence effect on bacterial pathogens.

### Example 9: Natural Transformation Effect

A key finding from the two Yu et al. studies was that ace-K could promote natural transformation and as such promote the acquisition of antibiotic resistance genes (Yu et al. (2021a); Yu et al. (2021b)). This finding is contrary to the transcriptomic data obtained by the present applicant. To investigate further, the impact of ace-K on natural transformation on the multidrug resistant strain of A. baumannii AB5075 was tested. Remarkably and in accordance with the applicant's transcriptomic data and motility data, supplementation of growth media with ace-K led to a significant reduction in natural transformation.

The results are shown in FIG. 51. It was found that supplementation of media with 1.33% ace-K led to a significant reduction in transformation efficiency, in contrast to the findings of Yu et al. Data shown is average of five biological replicates with SD. Data analysis by students t test. *p≤0.05, **p≤0.01***p≤0.001 versus the bacterial transformation in control samples.

### Example 10: Cation Supplementation to Mitigate Growth Inhibition by Ace-K and Saccharin

The transcriptomic data suggested that the bacterial cell membrane may be significantly altered upon exposure to ace-K or saccharin. If either disrupts membrane permeability, it ought to be possible to mitigate this through the addition of exogenous cations. To explore this possibility the growth assays described above in Example 1 were repeated, but in media supplemented with magnesium and calcium cations. These cations are known to help maintain membrane stability.

A. baumannii AB5075 and P. aeruginosa clinical isolate G4R7 were grown in LB, LB including ace-K, and LB including ace-K and further supplemented with Mg²⁺ and Ca²⁺ cations. Remarkably (as can be seen in FIG. 52) the addition of cations to both A. baumannii AB5075 and P. aeruginosa G4R7 partially restored the growth inhibition observed in the presence of ace-K.

Given that ace-K had such a pronounced impact on the growth of P. aeruginosa and A. baumannii, and that the mechanism was through membrane disruption, it was hypothesised that it may have the same effect against other clinically relevant pathogens. To explore this, growth assays in the presence of 2.66% ace-K were conducted.

Eight different bacterial species (A. baumannii, P. aeruginosa, E. coli, Stenotrophomonas maltophilia, Klebsiella pneumoniae, Staphylococcus aureus, Enterococcus faecalis and Enterobacter cloacae) were grown in LB, LB including ace-K, and LB including ace-K and further supplemented with Mg²⁺ and Ca²⁺ cations.

The results are shown in FIG. 52. Data shown is average of three biological replicates with SD. Data analysis by students t test. *p≤0.05, **p≤0.01***p≤0.001 versus the LB control.

Remarkably, this assay demonstrated that ace-K significantly inhibits the growth of Enterococcus faecalis, Enterobacter cloacae, E. coli, Stenotrophomonas maltophilia, and Klebsiella pneumoniae, but not Staphylococcus aureus. The finding that both Gram-negative and Gram-positive bacteria were impacted by ace-K highlights the potency of its activity. S. aureus did have a minor reduction in growth in the presence of ace-K, but this was not significant.

The supplementation of the media with Mg²⁺ and Ca²⁺ cations was able to at least partially reverse the inhibitory effect of ace-K on P. aeruginosa, A. baumannii, E. coli, Stenotrophomonas maltophilia, Klebsiella pneumoniae and Enterobacter cloacae.

As membrane permeability can be mitigated through the addition of exogenous cations, the hypothesis that membrane disruption is responsible for ace-K effect on growth is supported by these data.

This effect was also demonstrated with saccharin and A. baumannii. The results are shown in FIG. 85 (N=3 biological triplicate; analysis by t-test), each with technical triplicates where it can be seen that supplementation with cations at least partially restores growth.

### Example 11: Antibiotic Potentiation Effect

The disruption of the membrane by ace-K suggests that bacteria may be rendered more susceptible to antibiotic treatment in the presence of ace-K. To explore this hypothesis, the susceptibility of A. baumannii and P. aeruginosa to a panel of different antibiotics in the presence and absence of a sub-minimum inhibitory concentration of ace-K was tested. P. aeruginosa was grown in the presence of ace-k for 19 hours, and exposed to commonly used antibiotics (gentamicin, piperacillin/tazobactam, and polymyxin B). FIG. 53 shows that this resulted in an increased susceptibility to gentamicin, piperacillin/tazobactam and polymyxin B. The data present the mean of three biological replicates±SD. *p≤0.05 versus the bacterial growth in control plates.

In the case of A. baumannii, ace-K potentiates the activity of gentamicin, polymyxin B, doripenem, imipenem and meropenem (FIGS. 54 and 55).

A. baumannii AB5075 is known to be resistant to carbapenems. It was grown on agar plates without ace-K, or with 2.2% or 2.4% ace-K. Discs impregnated with polymyxin B, gentamicin, meropenem, imipenem and doripenem were added to the plates. The results are shown graphically in FIG. 55 where exposure to ace-K led to significant increase in the size of the zone of clearance for each antibiotic. Minimum of three biological replicates for all except doripenem which only has one. Data analysis by students t test. *p≤0.05, **p≤0.01***p≤0.001 versus the control.

FIG. 54 shows a visual representation of zones of clearance of A. baumannii AB5075 around an E strip for doripenem, imipenem and meropenem with 0%, 2.2% or 2.4% Ace-K. The E strip is impregnated with a concentration gradient of the antibiotic with highest concentration at the top and lowest at the bottom. It can be seen that in the presence of ace-K a zone of inhibition is visible around the E strip for each of the antibiotics compared to the control which only has water added to the agar. The data presented is a representative image of three biological replicates±SD.

The presence of ace-K significantly increases the sensitivity of a multidrug resistant strain of A. baumannii AB5075 to aminoglycosides (such as gentamicin), polymyxins (such as polymyxin B), and beta-lactams, including carbapenems, (such as doripenem, meropenem, imipenem, and piperacillin).

The antibiotic potentiate effect was also seen for cyclamate and saccharin, where plates loaded with 2.66% of each sweetener and paper discs impregnated with antibiotics (imipenem and doripenem) were placed on the plate and the zone of clearance measured after 24 hours of growth. This was then compared to the control plate (FIG. 56).

The ability of saccharin to increase the sensitivity of A. baumannii to carbapenems (specifically doripenem, imipenem, and meropenem) was also demonstrated. The results (the point to which growth was inhibited on the strip) are presented below in Table 5, and also visually in FIG. 86 (N=3 biological replicates; analysis by t-test).

TABLE 5

| | Control | SAC 1% | SAC 1.5% |
|---|---|---|---|
| DOR | 18.67 ± 4.61 | 13.33 ± 2.31 ns | 0.46 ± 0.07 |
| | (mg/L) | (mg/L) | (mg/L)** |
| IMI | 21.33 ± 4.61 | 18.67 ± 4.61 ns | 1 ± 0.43 |
| | (mg/L) | (mg/L) | (mg/L)** |
| MER | ≥32 | 29.33 ± 4.61 ns | 0.54 ± 0.19 |
| | (mg/L) | (mg/L) | (mg/L)**** |

### Example 12: Biofilm Eradication

To assess the ability of ace-K to eradicate established biofilms, overnight cultures were diluted in 96-well plates to OD₆₀₀ 0.1 for P. aeruginosa PA14 and 0.05 for A. baumannii AB5075 in LB medium. Plates were incubated for 18 hours at 37° C. and 180 rpm to allow biofilms to form. Following incubation, growth medium was removed from the wells and biofilms were washed three times with 200 μL of sterile PBS to remove any unbound planktonic cells. Fresh LB medium supplemented with 8.85% ace-K or an appropriate vehicle control was added to the wells. Plates were incubated for a further 24 hours at 37° C. and 180 rpm. Following this treatment biofilms were stained with 0.1% crystal violet as detailed above. Reduction in biofilm was represented as a percentage reduction compared to the control. This treatment led to reduced total biofilm biomass in AB5075 and PA14 by 48.8% and 69.7% respectively (FIG. 57).

To assess the ability of artificial sweeteners to disperse biofilms, overnight cultures were diluted in 96-well plates to OD₆₀₀ 0.5-0.1 in growth medium. Plates were incubated for 18 h at 37° C. and 180 rpm to allow biofilms to form. Following incubation growth medium was removed and wells washed three times with 200 μl of sterile PBS to remove any unattached cells. Fresh growth medium supplemented with artificial sweetener or an appropriate vehicle control was added to the wells. Plates were incubated for a further 24 h at 37° C. and 180 rpm. Following this treatment biofilms were stained with 0.1% crystal violet as detailed above. Biofilm dispersal was represented as a percentage reduction compared with the control.

Bacterial biofilm dispersal results are shown in FIGS. 80, 81 and 82 (N=3 biological triplicate, each with technical triplicates; analysis by ANOVA with a post-hoc Dunnett's multiple comparison test to compare each treatment with the control)

### Example 13: Ace-K Downregulates pilA Expression

The RNA-seq data indicated that ace-K could lead to the down regulations of the expression of pilA. The expression from the PpilA promoter was measured using a miniTn7T-based insertion bearing a PpilA::gfp transcriptional fusion (AB5075/miniTn7T-zeo-pilA::gfpmut3). An AB5075/miniTn7T-zeo-gfpmut3 strain, bearing the empty miniTn7T backbone, was used as a control. Overnight cultures of strains bearing either the PpilA::gfp fusion or the empty transposon were diluted 1:100 (v/v) in fresh LB broth supplemented with 0, 0.33%, 0.66% or 1.33% ace-K, or a mock treatment. Cultures were incubated for 2 h at 37° C., 180 rpm. Then, samples were withdrawn from the cultures, washed with PBS and eventually resuspended in PBS. Two technical repeats of each sample were allocated in a 96-well plate and their OD₆₀₀ and GFP fluorescence (485 nm excitation, 535 nm emission) were measured. The fluorescence readings were normalised by their respective OD₆₀₀ and the baseline fluorescence obtained from the empty transposon control was subtracted from that obtained with the strain bearing the PpilA::gfpmut3 the promoter fusion measurements. A clear dose dependent response is seen for pilA expression upon ace-K exposure confirming the RNA-Seq data. Three biological replicates were performed for each experimental condition (FIG. 58). Analysis was by independent t-test between treated samples and the corresponding water control (***p=<0.001, ****p=<0.0001).

### Example 14: Effect on Twitching Motility is Dose Dependent and Non-Strain Specific

To determine if the impact on motility was dose dependent across different strains of bacteria, the assays described in Example 8 were repeated at a wider range of concentrations using two additional A. baumannii strains (AB0057 and BAA 747). As expected from Example 8, a dose-dependent decrease in the twitching motility of AB5075 over increasing concentrations of ace-K was observed. However this effect is not strain-specific. Other commonly used A. baumannii strains (AB0057 and BAA 747) also exhibited a dose-dependent decrease in twitching motility within the same range of ace-K concentrations (FIG. 59A-C). Results are represented as averages of five biological replicates #S.D. Statistical analysis was by independent t-test between treated samples and their corresponding water control (*p=<0.05, **p=<0.01, ***p=<0.001, ****p=<0.0001).

This effect was also observed in A. baumannii with saccharin, as shown in FIG. 87 (N=5 biological replicates; analysis by t-test).

### Example 15: Effect on Natural Transformation is Dose Dependent

To validate that the effect seen in Example 9 and demonstrate that it is not specific to a concentration of 1.33%, we repeated the natural transformation assay at a wider range of concentrations. Transformation frequency was impacted by ace-K in a dose dependent manner, with transformation being completely abolished at 0.66% and above. Furthermore, the drop-in transformation frequency occurred even in the presence of divalent cations (CaCl2 2 mM and MgSO4 1 mM), which are proven to increase natural transformation frequency in A. baumannii. In this condition, a significant drop in transformability was observed at 0.33% compared to the control, reaching transformation abolition at 0.66% ace-K and above. Also, as cell viability was not affected in the presence of cations when supplementing with ace-K, we discarded the possibility that this effect might be due to growth inhibition (FIG. 60A-C). Results are represented as averages of five biological replicates±S.D. Statistical analysis was by independent t-test between treated samples and their corresponding water control (*p=<0.05, **p=<0.01, ***p=<0.001, ****p=<0.0001).

### Example 16: Ace-K and Saccharin Increase Bacterial Cell Membrane Permeability

To explore the impacts of ace-K on the bacterial cell membrane, firstly its effect on membrane permeability was assessed using the membrane specific dye, Nile Red and the nucleic acid stain DAPI. Cultures of AB5075 of OD₆₀₀ 0.05 were prepared in 15 mL of either LB broth containing 1.33% ace-K or an equivalent volume of water in a 100 ml Erlenmeyer flask. Cultures were incubated at 37° C. and 180 rpm shaking for two hours. Following incubation 10 μL of a 1 mg/mL DAPI solution and 10 μL of a 5 mg/mL solution of Nile Red were added to each flask before returning to the incubator for 30 mins. Once stained, cultures were centrifuged at 5000 rpm for five minutes and the supernatant discarded. Pellets were resuspended in 10 ml of sterile 4% formaldehyde in PBS and incubated in the dark for 30 mins to fix. Once fixed, samples were centrifuged at 5000 rpm and pellets were washed twice with 10 mL sterile PBS. After washing pellets were resuspended in 10 ml of sterile PBS and 10 μL of the cell suspension was spotted onto a glass slide and allowed to air dry in the dark. Three spots were prepared per flask. A cover slip was affixed to the slide and samples were imaged using Leica HF14 DM4000 microscope using CY3 (Ex: 542-568 nm, Em: 579-631 nm) and DAPI (Ex 325-375 nm, Em: 435-485 nm) filters. This assay confirmed that when grown in the presence of a sub-MIC of ace-K, A. baumannii AB5075 showed significant increases in nuclear dye uptake as compared to the untreated control (FIG. 61) indicating a more permeable membrane.

The membranes of A. baumannii AB5075, K. pneumoniae, and E. coli were also all significantly permeabilised. This was demonstrated by increased staining with DAPI compared to a control (see FIGS. 88 to 90).

### Example 17: Ace-K Triggers Gross Morphological Changes and Leads to Cell Envelope Bulges

Live cell imaging was used to monitor the impact of ace-K on the cell over time. It was observed that A. baumannii cells stop dividing and lose structural integrity, swelling in size rapidly, upon ace-K exposure. We also observed the formation of bulges in the bacterial cell. Using the Cardiolipin (CL)-specific fluorescent dye 10-N-nonyl-acridine orange (NAO) to visualise CL distribution, clear structural rearrangements in the phospholipid composition of the cell membrane are visible and it is possible to visualise that the bulges were emerging from cells (FIG. 62A). The live cell imaging was repeated using the carbapenem resistant E. coli NCTC 13476. A conserved loss of morphology was seen, but distinct from that seen in A. baumannii, E. coli cells filamented, extending to many times their original size before eventually, forming characteristic membrane bulges and ultimately lysing (FIG. 62B). To visualise the localisation of these membrane bulges and to gain insight into the contents of the bulges, an E. coli MG1655 strain with labelled mCherry-Fis and CFP-FtsZ was used (FIG. 62CD). Fis is a small DNA-binding protein that binds to a large number of regions of the chromosome, allowing the visualisation of the nucleosome in living cells. FtsZ is a component of the Z ring, showing future cell division sites. This time lapse experiment revealed that the membrane bulges were largely localised to either a site where a septum is formed or at a site where invagination has already taken place. The mCherry-Fis also confirmed that these bulges contain nuclear material. This indicates that the mechanism by which ace-K is leading to cell death is through bulge mediated cell lysis.

### Example 18: Ace-K can Potentiate Antibiotic Activity in Ex Vivo Model

The potential of ace-k as part of a combination therapy was determined using the same porcine ex vivo burn wound model described in Example 1. Burn wound biofilms were treated with either gauze loaded with 1.5 mL of 0.59 mg/mL polymyxin B singularly (a dose equivalent to the commonly used, polymyxin containing topical cream Neosporin) or polymyxin B in combination with ace-K. Burn wound biofilms treated with polymyxin B showed a 2.11 log reduction in viable cells while treatment with a combination of polymyxin B and ace-K showed an improved log reduction of 3.13 (FIG. 63). Data shown represents the average of three biological replicates±S.D. (*p=<0.05, **p=<0.01, ***p=<0.001, ****p=<0.0001).

The effect of ace-K in combination with polymyxin is therefore far greater than polymyxin alone.

### Example 19: Preparation of a Sweetener-loaded Hydrogel

A method of preparing a hydrogel loaded with a sweetener, in this example, saccharin, is illustrated in FIG. 91. Sodium saccharin (8% (w/w)), potassium tetraborate (3% (w/w)), and PVA (3% ((w/w)) were added into a beaker and mixed with deionized water. The mixture was placed in a water bath and heated at 80° C. for three hours with watch glass while being swirled with a stirring rod every 1 hour. Once hydrogels had been obtained, they were placed in a petri dish with custom-made moulds. The therapeutic potential of saccharin hydrogel was determined using the same porcine ex vivo burn wound model described in Example 1, and the results are shown in FIG. 92.

The applicant has demonstrated that artificial sweeteners, such as xylitol, mannitol, erythritol, cyclamate, sorbitol, sucralose, maltitol, lactitol monohydrate, sodium saccharin and ace-K, can inhibit the growth of a range of the most clinically relevant pathogens. They can also inhibit a range of different virulence associated behaviours in those pathogens.

Remarkably some of these sweeteners have been shown to augment the efficacy of a range of antibiotics in clinical use. The presence of the sweetener in an amount that would not have an antibacterial effect by itself renders a bacterium sensitive to antibiotic that it would otherwise be resistant to. Without being bound by theory, the sweetener may be having an effect on the bacterium's antibiotic resistance mechanisms. For example, it may disable or reduce the expression of an enzyme that digests the antibiotic, block or disrupt antibiotic efflux pumps, or change the structure of the bacterial cell membrane enabling the antibiotic to enter.

The therapeutic application of these sweeteners could have a major impact on tackling infection, in particular multidrug resistant infections. The skilled person will appreciate that derivatives of the exemplified artificial sweeteners, pharmaceutically acceptable salts of the artificial sweeteners, and compounds related to the artificial sweeteners could equally be useful in the compositions and methods described herein.

Given that these sweeteners, in particular ace-K, saccharin and cyclamate, are already present in the diet at relatively high concentrations, the potential to repurpose these compounds as therapeutic agents is promising. The applicant has not identified any previous disclosure relating to the use of, in particular, ace-K or cyclamate as antibacterial therapeutics.

Although preferred embodiments have been described in the context of human healthcare, the skilled person will appreciate that the artificial sweeteners and compositions could also be administered to animals in need thereof. Embodiments of the invention thus also find use in veterinary care.

Furthermore, the above Examples have been carried out using well known artificial sweeteners. The skilled person will appreciate that modifications to the structures of these sweeteners could be made without affecting activity, or even to improve their activity. The skilled person will thus appreciate that chemically related derivatives of the described sweeteners could equally be used.

By way of example, additional sulphonamide groups could be added to ace-K, cyclamate or saccharin potentially to augment their antibacterial activity.

Sweeteners could also be conjugated to a secondary compound to maximise the activity of either the sweetener or the secondary compound. For example, a sweetener could be conjugated to an antibiotic to enhance the above-described antibiotic potentiation effect.

Other modifications could help target the sweetener to the cell, for example by linking it to a siderophore or a quorum sensing signalling molecule such as an acyl homoserine lactone. These modifications could increase the concentration of the sweetener around the bacterial cells and potentially facilitate internalisation of the sweetener.

Other possible modifications could include substituted benzotriazole derivatives, alternate metal complexing ions, introducing electron withdrawing groups into the benzene ring or modifications that increase ionization of an N—H group, or degradative intermediates such as benzoic acid, 2-(1-oxopropyl)-, 1-phenoxyphthalazine, methcathinone, 1,2,3-benzenetricarboxylic acid trimethyl ester, 1-dyrrol [tert-buty(dimethyl)sily]oxymorphopropan-2-ol and terephthalic acid di (2-methoxyethyl) ester).

All optional and preferred features and modifications of the described embodiments and dependent claims are usable in all aspects of the invention taught herein. Furthermore, the individual features of the dependent claims, as well as all optional and preferred features and modifications of the described embodiments are combinable and interchangeable with one another.

The disclosures in United Kingdom patent applications 2205515.6 and 2215010.6, from which this application claims priority, and in the accompanying abstract are incorporated herein by reference.

### REFERENCES

Bian et al. (2017) PLOS ONE 12, e0178426
HM Government (2019) “Tackling antimicrobial resistance 2019-2024: The UK's five-year national action plan” Dept. of Health and Social Care, policy paper.
Mahmud et al. (2019) J. Mol. Microbiol. Biotechnol. 29, 43-56
Markus et al. (2021) Int. J. Mol. Sci. 22, 9863
Murray et al. (2022) Lancet 399, 629-55
Shahriar et al. (2020) Metabol. Open 8, 100072
Wang et al. (2018) PLOS ONE 13, e0199080
Yu et al. (2021a) ISME J. 15, 2117-30
Yu et al. (2021b) ISME J. 16, 543-54

1. A composition including an active agent in an amount sufficient to inhibit bacterial growth and/or virulence, wherein the active agent is an artificial sweetener or a chemically related derivative thereof, for use in a method of treating and/or preventing infection.

2. A composition including an active agent in an amount sufficient to inhibit bacterial growth and/or virulence, wherein the active agent is ace-K, saccharin, cyclamate, sucralose, a sugar alcohol or a chemically related derivative thereof, for use in a method of treating and/or preventing infection.

3. A composition for use as claimed in claim 1, wherein the composition includes ace-K, saccharin, cyclamate, sucralose, a sugar alcohol or a chemically related derivative thereof.

4. A composition for use as claimed in claim 1, 2 or 3, wherein the active agent has a structure that includes a sulphonamide group.

5. A composition for use as claimed in claim 2 or 3, wherein the sugar alcohol is xylitol, mannitol, sorbitol, erythritol, maltitol and/or lactitol, or a chemically related derivative thereof.

6. A composition for use as claimed in any of claims 1 to 4, wherein the active agent is ace-K, saccharin or cyclamate, or a chemically related derivative thereof.

7. A composition for use as claimed in any preceding claim, wherein the infection is a bacterial infection caused by Pseudomonas aeruginosa, Acinetobacter baumannii, Staphylococcus aureus, Enterococcus faecalis, Klebsiella pneumoniae, Stenotrophomonas maltophilia, and/or Enterobacter species.

8. A composition for use as claimed in any preceding claim, wherein the infection is a bacterial infection caused by Pseudomonas aeruginosa, Acinetobacter baumannii, Staphylococcus aureus, Stenotrophomonas maltophilia, and/or Enterobacter species.

9. A composition for use as claimed in any preceding claim, wherein the infection is a bacterial infection caused by Pseudomonas aeruginosa and/or Acinetobacter baumannii.

10. A composition for use as claimed in any preceding claim, wherein biofilm formation is inhibited.

11. A composition for use as claimed in any preceding claim, wherein bacterial motility is inhibited.

12. A composition for use as claimed in any preceding claim, wherein the bacterial membrane is disrupted.

13. A composition including an active agent, wherein the active agent is ace-K, cyclamate, saccharin, or a chemically related derivative thereof, for use in increasing susceptibility of bacteria to antibiotic treatment.

14. A composition for use as claimed in claim 13, wherein the composition renders an antibiotic-resistant bacterium sensitive to the antibiotic.

15. A composition for use as claimed in claim 13 or 14, wherein the composition is for co-administration with an antibiotic.

16. A composition for use as claimed in claim 13, 14 or 15, wherein the composition includes an antibiotic.

17. A composition for use as claimed in any of claims 13 to 16, wherein the antibiotic is a beta-lactam antibiotic.

18. A composition for use as claimed in any of claims 13 to 17, wherein the antibiotic is a carbapenem antibiotic.

19. A composition for use as claimed in any of claims 13 to 18, wherein the antibiotic is an aminoglycoside antibiotic

20. A composition for use as claimed in any of claims 13 to 19, wherein the antibiotic is a polymyxin.

21. A composition for use as claimed in any preceding claim, wherein the active agent is present below a minimum inhibitory concentration.

22. A composition for use as claimed in any preceding claim, wherein the active agent is present at less than 15% (w/v).

23. A composition for use as claimed in any preceding claim, wherein the active agent is present at less than 10% (w/v).

24. A composition for use as claimed in any preceding claim, wherein the active agent is present at less than 5% (w/v).

25. A composition for use as claimed in any preceding claim, wherein the active agent is present at 3% (w/v) or less than 3% (w/v).

26. A composition for use as claimed in any preceding claim, wherein the active agent is present at 1.5% (w/v) or less than 1.5% (w/v).

27. A composition for use as claimed in any preceding claim, wherein the active agent is present at 1% (w/v) or less than 1% (w/v).

28. A composition for use as claimed in any preceding claim, wherein the composition is formulated for application to a patient's skin.

29. A composition for use as claimed in claim 28, in the form of a liquid, cream, ointment, gel or hydrogel

30. A wound dressing including a composition as claimed in claim 28 or 29.

31. A composition for use as claimed in any of claims 1 to 27, wherein the composition is formulated for inhalation.

32. A composition for use as claimed in claim 31, wherein the composition is in aerosolised or dry powder form.

33. A composition for use as claimed in any of claims 1 to 27, wherein the composition is formulated for intravenous administration.

34. A composition for use as claimed in any of claims 1 to 27, wherein the composition is formulated for oral administration.

35. A method of preventing and/or treating infection, including providing a composition including an artificial sweetener or a chemically related derivative thereof in an amount sufficient to inhibit bacterial growth and/or disable a virulence mechanism, and administering the composition to a patient in need thereof.

36. A method of treating or preventing a skin infection, including applying a composition as claimed in claim 28 or 29 or a wound dressing as claimed in claim 30 to a skin wound of a human or animal subject.

37. A method as claimed in claim 36, wherein the wound is a burn or laceration.

38. A method of treating or preventing an infection associated with lung disease, including providing to a human or animal subject a composition as claimed in claim 31 or 32.

39. A method of treating or preventing bacteraemia and/or sepsis, including providing to a human or animal subject a composition as claimed in claim 33 or 34.

40. A method of increasing the susceptibility of bacteria to antibiotic treatment including:
providing a composition including an active agent, wherein the active agent is ace-K, cyclamate, saccharin, or a chemically related derivative thereof;
providing an antibiotic; and
administering the composition and the antibiotic to a subject in need thereof.)

41. A method as claimed in claim 40, wherein the bacteria are resistant to the antibiotic, and the composition renders the bacteria sensitive to the antibiotic.

42. A method as claimed in claim 40 or 41, wherein the composition and the antibiotic are co-administered to the subject.

43. A method as claimed in claim 40, 41, or 42, wherein the composition includes the antibiotic.

44. A method as claimed in any of claims 35-43, wherein the composition is as claimed in any of claims 1 to 34.
